SEQUENCE LISTING (39 sequences)

SEQ ID NO: 1 - protein, 516 aa, Artificial Sequence
  Description of Artificial Sequence Synthetic polypeptide
  cannabidiolic-acid synthase; A6P6V9.1; signal peptide removed
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 2 - protein, 517 aa, Artificial Sequence
  Description of Artificial Sequence Synthetic polypeptide
  tetrahydrocannabinolic acid synthase; AB057805.1; secretion signal removed
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 3 - protein, 395 aa, Artificial Sequence
  Description of Artificial Sequence Synthetic polypeptide
  CBGAS; AJN57774.1
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 4 - protein, 307 aa, Artificial Sequence [sequence omitted]

SEQ ID NO: 5 - protein, 397 aa, Artificial Sequence
  Description of Artificial Sequence Synthetic polypeptide
  IspDF protein
MIALQRSLSMHVTAIIAAAGEGRRLGAPLPKQLLDIGGRSILERSVMAFARHERIDDVIVVLPPALAAAPPDWIAASGRVPAVHVVSGGERRQDSVANAFDRVPAQSDVVLVHDAARPFVTAELISRAIDGAMQHGAAIVAVPVRDTVKRVDPDGEHPVITGTIPRDTIYLAQTPQAFRRDVLGAAVALGRSGVSATDEAMLAEQAGHRVHVVEGDPANVKITTSADLDQARQRLRSAVAARIGTGYDLHRLIEGRPLIIGGVAVPCDKGALGHSDADVACHAVIDALLGAAGAGNVGQHYPDTDPRWKGASSIGLLRDALRLVQERGFTVENVDVCVVLERPKIAPFIPEIRARIAGALGIDPERVSVKGKTNEGVDAVGRGEAIAAHAVALLSES

SEQ ID NO: 6 - protein, 448 aa, Artificial Sequence
  Description of Artificial Sequence Synthetic polypeptide
  MFS aromatic acid antiporter
MNQAQTNVGKSLDVQSFINQQPLSRYQWRVVLLCFLIVFLDGLDTAAMGFIAPALSQEWGIDRASLGPVMSAALIGMVFGALGSGPLADRFGRKGVLVGAVLVFGGFSLASAYATNVDQLLVLRFLTGLGLGAGMPNATTLLSEYTPERLKSLLVTSMFCGFNLGMAGGGFISAKMIPAYGWHSLLVIGGVLPLLLALVLMIWLPESARFLVVRNRGTDKVRKTLSPIAPQVVAEAGSFSVPEQKAVAARNVFAVIFSGTYGLGTVLLWLTYFMGLVIVYLLTSWLPTLMRDSGASMEQAAFIGALFQFGGVLSAVGVGWAMDRFNPHKVIGIFYLLAGVFAYAVGQSLGNITLLATLVLVAGMCVNGAQSAMPSLAARFYPTQGRATGVSWMLGIGRFGAILGAWSGATLLGLGWSFEQVLTALLVPAALATVGVVVKGLVSHADAT

SEQ ID NO: 7 - protein, 420 aa, Artificial Sequence
  Description of Artificial Sequence Synthetic polypeptide
  OprD family porin
MSIAFKKTLACSATLLVAPYASAAFVEDFKGSLELRNFYYNRDFRNDGATQSKRDEWAQGFILNLQSGFTEGPVGFGIDAMGLLGVKLDSSPDRTGSGLLAYDSDRQVEDEYGKFVATAKARMGKTELRIGGVNPLMPLLWSNNSRLLPQVFRGGSLTVNDIDKLTVTATRINAVKQRNSTDFESLTATGYAPVEADHYNYLAFDFKPAKDMTFSLHAAELEDLYKSYFAGIKVIKPLWEGNVIADVRVFDASETGSKKLGEVDNRTLSSYFAYSIKGHTIGGGYQKAWGDTSFAFVNGTDTYLFGESLVSTFTAPEERVWFARYDFDFAALGVPGLLFTTRYMKGDDVNPDLLTSRQAASLRLNGEDGKEWERVTDISYVIQSGPAKGVSFQWRNSTNRSTYADSANENRLIMRYTFNF

SEQ ID NO: 8 - protein, 448 aa, Artificial Sequence
  Description of Artificial Sequence Synthetic polypeptide
  MFS aromatic acid antiporter
MNQAQTNVGKSLDVQSFINQQPLSRYQWRVVLLCFLIVFLDGLDTAAMGFIAPALSQEWGIDRASLGPVMSAALIGMVFGALGSGPLADRFGRKGVLVGAVLVFGGFSLASAYATNVDQLLVLRFLTGLGLGAGMPNATTLLSEYTPERLKSLLVTSMFCGFNLGMAGGGFISAKMIPAYGWHSLLVIGGVLPLLLALVLMVWLPESARFLVVRNRGTDKVRKTLSPIAPQVVAEAGSFSVPEQKAVAARNVFAVIFSGTYGLGTVLLWLTYFMGLVIVYLLTSWLPTLMRDSGASMEQAAFIGALFQFGGVLSAVGVGWAMDRFNPHKVIGIFYLLAGVFAYAVGQSLGNITLLATLVLVAGMCVNGAQSAMPSLAARFYPTQGRATGVSWMLGIGRFGAILGAWSGATLLGLGWSFEQVLTALLVPAALATVGVVVKGLVSHADAT

SEQ ID NO: 9 - protein, 420 aa, Artificial Sequence
  Description of Artificial Sequence Synthetic polypeptide
  OprD family porin
MSIAFKKTLACSATLLVAPYASAAFVEDFKGSLELRNFYYNRDFRNDGATQSKRDEWAQGFTLNLQSGFTEGPVGFGIDAMGLLGVKLDSSPDRTGSGLLAYDSDRQVEDEYGKFVATAKARMGKTELRIGGVNPLMPLLWSNNSRLLPQIFRGGSLTVNDIDKLTVTATRVNAVKQRNSTDFESLTATGYAPVEADHYNYLAFDFKPAKDMTFSLHAAELEDLYKSYFAGIKVIKPLWEGNVIADVRVFDASETGSKKLGEVDNRTLSSYFAYSIKGHTIGGGYQKAWGDTSFAFVNGTDTYLFGESLVSTFTAPEERVWFARYDFDFAALGVPGLLFTTRYMEGDDVNPDLLTSRQAASLRLNGEDGKEWERVTDISYVIQSGPAKGVSFQWRNSTNRSTYADSANENRLIMRYTFNF

SEQ ID NO: 10 - protein, 529 aa, Artificial Sequence
  Description of Artificial Sequence Synthetic polypeptide
  IspDE bifunctional MEP pathway enzyme
MATTHLDVCAVVPAAGFGRRMQTECPKQYLSIGNQTILEHSVHALLAHPRVKRVVIAISPGDSRFAQLPLANHPQITVVDGGDERADSVLAGLKAAGDAQWVLVHDAARPCLHQDDLARLLALSETSRTGGILAAPVRDTMKRAEPGKNAIAHTVDRNGLWHALTPQFFPRELLHDCLTRALNEGATITDEASALEYCGFHPQLVEGRADNIKVTRPEDLALAEFYLTRTIHQENTSLGGGGSAAAMRTQWPSPAKLNLFLYITGQRADGYHTLQTLFQFLDYGDTISIELRDDGDIRLLTPVEGVEHEDNLIVRAARLLMKTAADSGRLPTGSGANISIDKRLPMGGGLGGGSSNAATVLVALNHLWQCGLSMDELAEMGLTLGADVPVFVRGHAAFAEGVGEILTPVDPPEKWYLVAHPGVSIPTPVIFKDPELPRNTPKRSIETLLKCEFSNDCEVIARKRFREVDAVLSWLLEYAPSRLTGTGACVFAEFDTESEARQVLEQAPEWLNGFVAKGANLSPLHRAML

SEQ ID NO: 11 - protein, 10 aa, Artificial Sequence
  Description of Artificial Sequence Synthetic peptide
SLGGGGSAAA

SEQ ID NO: 12 - protein, 231 aa, Artificial Sequence
  Description of Artificial Sequence Synthetic polypeptide
MIALQRSLSMHVTAIIAAAGEGRRLGAPLPKQLLDIGGRSILERSVMAFARHERIDDVIVVLPPALAAAPPDWIAASGRVPAVHVVSGGERRQDSVANAFDRVPAQSDVVLVHDAARPFVTAELISRAIDGAMQHGAAIVAVPVRDTVKRVDPDGEHPVITGTIPRDTIYLAQTPQAFRRDVLGAAVALGRSGVSATDEAMLAEQAGHRVHVVEGDPANVKITTSADLDQA

SEQ ID NO: 13 - protein, 156 aa, Artificial Sequence
  Description of Artificial Sequence Synthetic polypeptide
RIGTGYDLHRLIEGRPLIIGGVAVPCDKGALGHSDADVACHAVIDALLGAAGAGNVGQHYPDTDPRWKGASSIGLLRDALRLVQERGFTVENVDVCVVLERPKIAPFIPEIRARIAGALGIDPERVSVKGKTNEGVDAVGRGEAIAAHAVALLSES

SEQ ID NO: 14 - protein, 234 aa, Artificial Sequence
  Description of Artificial Sequence Synthetic polypeptide
MQVTAIIAAGGRGRRFGGGVPKQLVGVGGRPILERTVAAFLGHPAIHEVVVALPAELMADPPAYLRAAPKPIRLVAGGVQRQDSVRQAFQAANEQSDVIVIHDAARPFASADLISRTIAAAAEGGAALAAVPARDTVKRGAFAAGRTGPAGRQAVEGAPLLVVAETLPRDSIYLAQTPQAFRRDVLRDALALGEAGSEATDEATLAERAGHIVRLVEGEPANIKITTPDDLLVA

SEQ ID NO: 15 - protein, 156 aa, Artificial Sequence
  Description of Artificial Sequence Synthetic polypeptide
FRIGAGYDLHRLVEGRPLVLGGVTIPFERGLLGHSDADAICHAVTDAVLGAAAAGDIGRHFPDSDPKWRDWSSIDLLRRASAIVKGRGYAIANVDAVVIAERPKLAPFLDEMRANVAGAIGIAVDAVGIKGKTNEGLGELGRGEAIAVHAVALLHL

SEQ ID NO: 16 - protein, 214 aa, Artificial Sequence
  Description of Artificial Sequence Synthetic polypeptide
MVHVSAIIAAGGRGERFGGPQPKQLLLLGGVPILKRTVDAFLRGYPFIEVIVALPAEFVANPPDYLDDVIVVEGGARRQDSVANAFRAVAPSAQVVVIHDAARPLVTPSLIERTVDAAVKHGAAIAALRATDTVKRGDASRVIRGTLPRDEIFLAQTPQAFRAGVLRDALALAASAADATDEAMLAEQAGHHVRLVDGDPRNLKITTPEDLEMA

SEQ ID NO: 17 - protein, 156 aa, Artificial Sequence
  Description of Artificial Sequence Synthetic polypeptide
RIGNGYDLHRLVTGRPLVLGGVTIPFEKGLQGHSDADAVCHAITDAILGAASAGDIGRHFPDTDPAWKDAKSIVLLQQAAQIVSRAGYAIANLDVVVIAQQPKLVPHIDAIRHSVAHALGIDVQQVSVKGKTNEGVDSMGAGESIAVHAVALLQHS

SEQ ID NO: 18 - protein, 394 aa, Artificial Sequence
  Description of Artificial Sequence Synthetic polypeptide
MATTHLDVCAVVPAAGFGRRMQTECPKQYLSIGNQTILEHSVHALLAHPRVKRVVIAISPGDSRFAQLPLANHPQITVVDGGDERADSVLAGLKAAGDAQWVLVHDAARPCLHQDDLARLLALSETSRTGGILAAPVRDTMKRAEPGKNAIAHTVDRNGLWHALTPQFFPRELLHDCLTRALNEGATITDEASALEYCGFHPQLVEGRADNIKVTRPEDLALAEFYLTLPTPSFEIRIGHGFDVHAFGGEGPIIIGGVRIPYEKGLLAHSDGDVALHALTDALLGAAALGDIGKLFPDTDPAFKGADSRELLREAWRRIQAKGYTLGNVDVTIIAQAPKMLPHIPQMRVFIAEDLGCHMDDVNVKATTTEKLGFTGRGEGIACEAVALLIKATK

SEQ ID NO: 19 - protein, 394 aa, Artificial Sequence
  Description of Artificial Sequence Synthetic polypeptide
MATTHLDVCAVVPAAGFGRRMQTECPKQYLSIGNQTILEHSVHALLAHPRVKRVVIAISPGDSRFAQLPLANHPQITVVDGGDERADSVLAGLKAAGDAQWVLVHDAARPCLHQDDLARLLALSETSRTGGILAAPVRDTMKRAEPGKNAIAHTVDRNGLWHALTPQFFPRELLHDCLTRALNEGATITDEASALEYCGFHPQLVEGRADNIKVTRPEDLALAEFYLSLGGGGSAAAIGHGFDVHAFGGEGPIIIGGVRIPYEKGLLAHSDGDVALHALTDALLGAAALGDIGKLFPDTDPAFKGADSRELLREAWRRIQAKGYTLGNVDVTIIAQAPKMLPHIPQMRVFIAEDLGCHMDDVNVKATTTEKLGFTGRGEGIACEAVALLIKATK

SEQ ID NO: 20 - protein, 413 aa, Artificial Sequence
  Description of Artificial Sequence Synthetic polypeptide
MATTHLDVCAVVPAAGFGRRMQTECPKQYLSIGNQTILEHSVHALLAHPRVKRVVIAISPGDSRFAQLPLANHPQITVVDGGDERADSVLAGLKAAGDAQWVLVHDAARPCLHQDDLARLLALSETSRTGGILAAPVRDTMKRAEPGKNAIAHTVDRNGLWHALTPQFFPRELLHDCLTRALNEGATITDEASALEYCGFHPQLVEGRADNIKVTRPEDLALAEFYLAEAAAKEAAAKEAAAKEAAAKEAAAKAAAIGHGFDVHAFGGEGPIIIGGVRIPYEKGLLAHSDGDVALHALTDALLGAAALGDIGKLFPDTDPAFKGADSRELLREAWRRIQAKGYTLGNVDVTIIAQAPKMLPHIPQMRVFIAEDLGCHMDDVNVKATTTEKLGFTGRGEGIACEAVALLIKATK

SEQ ID NO: 21 - protein, 394 aa, Artificial Sequence
  Description of Artificial Sequence Synthetic polypeptide
MATTHLDVCAVVPAAGFGRRMQTECPKQYLSIGNQTILEHSVHALLAHPRVKRVVIAISPGDSRFAQLPLANHPQITVVDGGDERADSVLAGLKAAGDAQWVLVHDAARPCLHQDDLARLLALSETSRTGGILAAPVRDTMKRAEPGKNAIAHTVDRNGLWHALTPQFFPRELLHDCLTRALNEGATITDEASALEYCGFHPQLVEGRADNIKVTRPEDLALAEFYLRQRLRSAVAAIGHGFDVHAFGGEGPIIIGGVRIPYEKGLLAHSDGDVALHALTDALLGAAALGDIGKLFPDTDPAFKGADSRELLREAWRRIQAKGYTLGNVDVTIIAQAPKMLPHIPQMRVFIAEDLGCHMDDVNVKATTTEKLGFTGRGEGIACEAVALLIKATK

SEQ ID NO: 22 - protein, 395 aa, Artificial Sequence
  Description of Artificial Sequence Synthetic polypeptide
MIALQRSLSMHVTAIIAAAGEGRRLGAPLPKQLLDIGGRSILERSVMAFARHERIDDVIVVLPPALAAAPPDWIAASGRVPAVHVVSGGERRQDSVANAFDRVPAQSDVVLVHDAARPFVTAELISRAIDGAMQHGAAIVAVPVRDTVKRVDPDGEHPVITGTIPRDTIYLAQTPQAFRRDVLGAAVALGRSGVSATDEAMLAEQAGHRVHVVEGDPANVKITTSADLDQADLPTPSFERIGTGYDLHRLIEGRPLIIGGVAVPCDKGALGHSDADVACHAVIDALLGAAGAGNVGQHYPDTDPRWKGASSIGLLRDALRLVQERGFTVENVDVCVVLERPKIAPFIPEIRARIAGALGIDPERVSVKGKTNEGVDAVGRGEAIAAHAVALLSES

SEQ ID NO: 23 - protein, 397 aa, Artificial Sequence
  Description of Artificial Sequence Synthetic polypeptide
MIALQRSLSMHVTAIIAAAGEGRRLGAPLPKQLLDIGGRSILERSVMAFARHERIDDVIVVLPPALAAAPPDWIAASGRVPAVHVVSGGERRQDSVANAFDRVPAQSDVVLVHDAARPFVTAELISRAIDGAMQHGAAIVAVPVRDTVKRVDPDGEHPVITGTIPRDTIYLAQTPQAFRRDVLGAAVALGRSGVSATDEAMLAEQAGHRVHVVEGDPANVKITTSADLDQASLGGGGSAAARIGTGYDLHRLIEGRPLIIGGVAVPCDKGALGHSDADVACHAVIDALLGAAGAGNVGQHYPDTDPRWKGASSIGLLRDALRLVQERGFTVENVDVCVVLERPKIAPFIPEIRARIAGALGIDPERVSVKGKTNEGVDAVGRGEAIAAHAVALLSES

SEQ ID NO: 24 - protein, 425 aa, Artificial Sequence
  Description of Artificial Sequence Synthetic polypeptide
MIALQRSLSMHVTAIIAAAGEGRRLGAPLPKQLLDIGGRSILERSVMAFARHERIDDVIVVLPPALAAAPPDWIAASGRVPAVHVVSGGERRQDSVANAFDRVPAQSDVVLVHDAARPFVTAELISRAIDGAMQHGAAIVAVPVRDTVKRVDPDGEHPVITGTIPRDTIYLAQTPQAFRRDVLGAAVALGRSGVSATDEAMLAEQAGHRVHVVEGDPANVKITTSADLDQARQRLRSAVLAEAAAKEAAAKEAAAKEAAAKEAAAKAAARIGTGYDLHRLIEGRPLIIGGVAVPCDKGALGHSDADVACHAVIDALLGAAGAGNVGQHYPDTDPRWKGASSIGLLRDALRLVQERGFTVENVDVCVVLERPKIAPFIPEIRARIAGALGIDPERVSVKGKTNEGVDAVGRGEAIAAHAVALLSES

SEQ ID NO: 25 - protein, 246 aa, Artificial Sequence
  Description of Artificial Sequence Synthetic polypeptide
MATTHLDVCAVVPAAGFGRRMQTECPKQYLSIGNQTILEHSVHALLAHPRVKRVVIAISPGDSRFAQLPLANHPQITVVDGGDERADSVLAGLKAAGDAQWVLVHDAARPCLHQDDLARLLALSETSRTGGILAAPVRDTMKRAEPGKNAIAHTVDRNGLWHALTPQFFPRELLHDCLTRALNEGATITDEASALEYCGFHPQLVEGRADNIKVTRPEDLALAEFYLTRTIHQENTSLGGGGSAAA

SEQ ID NO: 26 - protein, 283 aa, Artificial Sequence
  Description of Artificial Sequence Synthetic polypeptide
MRTQWPSPAKLNLFLYITGQRADGYHTLQTLFQFLDYGDTISIELRDDGDIRLLTPVEGVEHEDNLIVRAARLLMKTAADSGRLPTGSGANISIDKRLPMGGGLGGGSSNAATVLVALNHLWQCGLSMDELAEMGLTLGADVPVFVRGHAAFAEGVGEILTPVDPPEKWYLVAHPGVSIPTPVIFKDPELPRNTPKRSIETLLKCEFSNDCEVIARKRFREVDAVLSWLLEYAPSRLTGTGACVFAEFDTESEARQVLEQAPEWLNGFVAKGANLSPLHRAML

SEQ ID NO: 27 - protein, 452 aa, Artificial Sequence
  Description of Artificial Sequence Synthetic polypeptide
MRTQWPSPAKLNLFLYITGQRADGYHTLQTLFQFLDYGDTISIELRDDGDIRLLTPVEGVEHEDNLIVRAARLLMKTAADSGRLPTGSGANISIDKRLPMGGGLGGGSSNAATVLVALNHLWQCGLSMDELAEMGLTLGADVPVFVRGHAAFAEGVGEILTPVDPPEKWYLVAHPGVSIPTPVIFKDPELPRNTPKRSIETLLKCEFSNDCEVIARKRFREVDAVLSWLLEYAPSRLTGTGACVFAEFDTESEARQVLEQAPEWLNGFVAKGANLSPLHRAMLSLGGGGSAAAMRIGHGFDVHAFGGEGPIIIGGVRIPYEKGLLAHSDGDVALHALTDALLGAAALGDIGKLFPDTDPAFKGADSRELLREAWRRIQAKGYTLGNVDVTIIAQAPKMLPHIPQMRVFIAEDLGCHMDDVNVKATTTEKLGFTGRGEGIACEAVALLIKATK

SEQ ID NO: 28 - DNA, 27 bp, Artificial Sequence
  Description of Artificial Sequence Synthetic primer
ggatgtgctgcaaggcgattaagttgg

SEQ ID NO: 29 - DNA, 26 bp, Artificial Sequence
  Description of Artificial Sequence Synthetic primer
ctcgtatgttgtgtggaattgtgagc

SEQ ID NO: 30 - protein, 6 aa, Artificial Sequence
  Description of Artificial Sequence Synthetic 6xHis tag
HHHHHH

SEQ ID NO: 31 - protein, 29 aa, Artificial Sequence
  Description of Artificial Sequence Synthetic peptide
AEAAAKEAAAKEAAAKEAAAKEAAAKAAA

SEQ ID NO: 32 - protein, 7 aa, Artificial Sequence
  Description of Artificial Sequence Synthetic peptide
LPTPSFE

SEQ ID NO: 33 - protein, 10 aa, Artificial Sequence
  Description of Artificial Sequence Synthetic peptide
RQRLRSAVAA

SEQ ID NO: 34 - protein, 17 aa, Artificial Sequence
  Description of Artificial Sequence Synthetic peptide
EAIARGTGERAVGERAA

SEQ ID NO: 35 - protein, 13 aa, Artificial Sequence
  Description of Artificial Sequence Synthetic peptide
ERLIGARNTAGAM

SEQ ID NO: 36 - protein, 395 aa, Artificial Sequence
  Description of Artificial Sequence Synthetic polypeptide
MATTHLDVCAVVPAAGFGRRMQTECPKQYLSIGNQTILEHSVHALLAHPRVKRVVIAISPGDSRFAQLPLANHPQITVVDGGDERADSVLAGLKAAGDAQWVLVHDAARPCLHQDDLARLLALSETSRTGGILAAPVRDTMKRAEPGKNAIAHTVDRNGLWHALTPQFFPRELLHDCLTRALNEGATITDEASALEYCGFHPQLVEGRADNIKVTRPEDLALAEFYLTRTIHQENTMRIGHGFDVHAFGGEGPIIIGGVRIPYEKGLLAHSDGDVALHALTDALLGAAALGDIGKLFPDTDPAFKGADSRELLREAWRRIQAKGYTLGNVDVTIIAQAPKMLPHIPQMRVFIAEDLGCHMDDVNVKATTTEKLGFTGRGEGIACEAVALLIKATK

SEQ ID NO: 37 - protein, 407 aa, Artificial Sequence
  Description of Artificial Sequence Synthetic polypeptide
MQVTAIIAAGGRGRRFGGGVPKQLVGVGGRPILERTVAAFLGHPAIHEVVVALPAELMADPPAYLRAAPKPIRLVAGGVQRQDSVRQAFQAANEQSDVIVIHDAARPFASADLISRTIAAAAEGGAALAAVPARDTVKRGAFAAGRTGPAGRQAVEGAPLLVVAETLPRDSIYLAQTPQAFRRDVLRDALALGEAGSEATDEATLAERAGHIVRLVEGEPANIKITTPDDLLVAEAIARGTGERAVGERAAFRIGAGYDLHRLVEGRPLVLGGVTIPFERGLLGHSDADAICHAVTDAVLGAAAAGDIGRHFPDSDPKWRDWSSIDLLRRASAIVKGRGYAIANVDAVVIAERPKLAPFLDEMRANVAGAIGIAVDAVGIKGKTNEGLGELGRGEAIAVHAVALLHL

SEQ ID NO: 38 - protein, 383 aa, Artificial Sequence
  Description of Artificial Sequence Synthetic polypeptide
MVHVSAIIAAGGRGERFGGPQPKQLLLLGGVPILKRTVDAFLRGYPFIEVIVALPAEFVANPPDYLDDVIVVEGGARRQDSVANAFRAVAPSAQVVVIHDAARPLVTPSLIERTVDAAVKHGAAIAALRATDTVKRGDASRVIRGTLPRDEIFLAQTPQAFRAGVLRDALALAASAADATDEAMLAEQAGHHVRLVDGDPRNLKITTPEDLEMAERLIGARNTAGAMRIGNGYDLHRLVTGRPLVLGGVTIPFEKGLQGHSDADAVCHAITDAILGAASAGDIGRHFPDTDPAWKDAKSIVLLQQAAQIVSRAGYAIANLDVVVIAQQPKLVPHIDAIRHSVAHALGIDVQQVSVKGKTNEGVDSMGAGESIAVHAVALLQHS

SEQ ID NO: 39 - protein, 371 aa, Artificial Sequence
  Description of Artificial Sequence Synthetic polypeptide
MSEMSLIMLAAGNSTRFNTKVKKQFLRLGNDPLWLYATKNLSSFYPFKKIVVTSSNITYMKKFTKNYEFIEGGDTRAESLKKALELIDSEFVMVSDVARVLVSKNLFDRLIENLDKADCITPALKVADTTLFDNEALQREKIKLIQTPQISKTKLLKKALDQNLEFTDDSTAIAAMGGKIWFVEGEENARKLTFKEDLKKLDLPTPSFEIFTGNGFDVHEFGENRPLLLAGVQIHPTMGLKAHSDGDVLAHSLTDAILGAAGLGDIGELYPDTDMKFKNANSMELLKQAYDKVREIGFELINIDICVMAQSPKLKDFKQAMQSNIAHTLDLDEFRINVKATTTEKLGFIGRKEGMAVLSSVNLKYFDWTRL

(19) United States
(12) Patent Application Publication
(10) Pub. No.: US 2022/0170056 A1
(43) Pub. Date: Jun. 2, 2022

(54) COMPOSITIONS AND METHODS FOR BIOSYNTHESIS OF TERPENOIDS OR CANNABINOIDS IN A HETEROLOGOUS SYSTEM

(71) Applicant: INMED PHARMACEUTICALS INC., Vancouver (CA)

(72) Inventors: Vikramaditya G. YADAV, Vancouver (CA); Protiva ROY, Vancouver (CA); Sonal AYAKAR, Vancouver (CA); Benson CHANG, Vancouver (CA); Eric HSU, Vancouver (CA)

(21) Appl. No.: 17/436,548

(22) PCT Filed: Mar. 6, 2020
(86) PCT No.: PCT/CA2020/050309

§ 371 Date: Sep. 3, 2021

Related U.S. Application Data
(60) Provisional application No. 62/814,816, filed on Mar. 6, 2019.
(60) Provisional application No. 62/814,823, filed on Mar. 6, 2019.

(51) Int. Cl. C12P 7/42; C12N 1/20; C12N 1/06; C12N 15/70; C12N 15/52; C12N 9/10; C12N 9/12; C12N 9/02; C12N 9/88; C12P 5/02; C07K 14/705; C12N 9/00

(52) U.S. Cl. CPC C12P 7/42; C12N 2500/30; C12N 1/06; C12N 15/70; C12N 15/52; C12N 9/1085; C12Y 205/01; C12Y 205/01001; C12N 9/1241; C12Y 207/0706; C12N 9/0004; C12Y 121/03008; C12N 9/88; C12Y 402/03; C12P 5/026; C07K 14/705; C12N 9/00; C12N 1/20

(57) ABSTRACT

Provided herein are methods and compositions for producing cannabinoids and other metabolites in a host cell.

### CROSS-REFERENCES TO RELATED APPLICATIONS

This application claims priority to U.S. Provisional Application No. 62/814,823, filed Mar. 6, 2019; and U.S. Provisional Application No. 62/814,816, filed Mar. 6, 2019, the contents of each of which are hereby incorporated in the entirety for any and all purposes.

### BACKGROUND OF THE INVENTION

Cannabinoids, and derivatives thereof, have several properties with therapeutic potential. Activation or blocking of CB-1 and/or CB-2 receptors with a cannabinoid can regulate downstream signaling and metabolic pathways and subsequently influence synaptic transmission, including transmission of pain and other sensory signals in the periphery, immune response, and inflammation. Thus, there is an interest in the use of natural or synthetic cannabinoids for therapeutic purposes. However, low extraction yields, and high separation costs have rendered the use of naturally-derived cannabinoids uneconomical. Similarly, fully synthetic methods of cannabinoid production are hampered by the complexity of these compounds.

Heterologous systems for production of cannabinoids known in the art rely on eukaryotic host organisms for production and secretion of cannabinoid synthase enzymes, which are then used to produce a cannabinoid product in an in vitro enzyme-catalyzed reaction. For example, U.S. Pat. Nos. 9,587,212; 9,512,391; 9,394,512; 9,526,715; 9,359,625 each describe methods and compositions and bioreactors for making cannabinoids in vitro using a recombinant Pichia pastoris that secretes THCA synthase or CBDA synthase. Unfortunately, however, this system requires the use of a eukaryotic host and additional means to generate a suitable substrate for the secreted enzyme.

With respect to in vivo cannabinoid production schemes, Carvalho Â, et al. FEMS Yeast Res. 2017, teaches that prokaryotic production of enzymes in the late cannabinoid pathway is not feasible due requirements of these enzymes for membrane association, glycosylation, and disulfide bond formation. In particular, Carvalho discloses that expression of CBGAS in E. coli is rather unlikely and that the use of a prokaryotic host to express functional THCAS or CBDAS is excluded.

Moreover, olivetolate a substrate of the aromatic prenyltransferase CBGAS required for production of CBGA is not endogenously produced at useful levels, if at all, in common prokaryotic systems. As such, the olivetolate must be supplied exogenously to the culture media of the cell or by expression of yet another biosynthetic pathway for heterologous production of olivetolate. However, biosynthetic production of olivetolate is a metabolic burden that can dramatically reduce microbial output. Similarly, olivetolate is not efficiently transported into the cell from the surrounding media and therefore exogenously supplied olivetolate presents a rate limiting step in the production of down-stream metabolites. Other aromatic prenyltransferase substrates such as divarinolic acid (DVA) encounter the same issues with respect to endogenous production, metabolic burden of heterologous production, and rate limiting membrane transport. Thus, there is a long felt and unmet need to develop a cost-effective heterologous system for the production of cannabinoids in vivo.

### SUMMARY OF THE INVENTION

Described herein are improved methods, compositions, and host cells for improved prenylation of aromatic substrates, or production of down-stream metabolites thereof, in a (e.g., prokaryotic) host cell. The present inventors have identified membrane transporters that are functional and capable of increasing the transport of extracellular aromatic prenyltranferase substrates such as olivetolate into the (e.g., prokaryotic) host cell when expressed as heterologous transporters in a host cell. For example, the present inventors have identified a major facilitator superfamily (MFS) aromatic acid antiporter that is functional and capable of increasing the transport of extracellular aromatic prenyltranferase substrates such as olivetolate into the (e.g., prokaryotic) host cell. Independently, the present inventors have identified an outer membrane porin (OMP) superfamily transporter that is functional and capable of increasing the transport of extracellular aromatic prenyltranferase substrates such as olivetolate into the (e.g., prokaryotic) host cell. Without wishing to be bound by theory, the present inventors hypothesize that the increased transport of aromatic prenyltransferase substrates such as olivetolate into the cell, e.g., via an antiporter or porin, increases flux through the aromatic prenylation step and thereby improves production of down-stream metabolic products. In some cases, the increased flux decreases the (e.g., steady state) intracellular concentration of toxic intermediates such as geranylpyrophosphate (GPP) and thereby improves production of down-stream metabolic products.

Thus, the present invention provides a host cell comprising: a) an expression cassette comprising a promoter operably linked to a heterologous nucleic acid encoding a transporter; and, and b) an exogenous aromatic substrate of the transporter. In embodiments, the host cell is capable of increased import of an aromatic substrate of the transporter into the host cell as compared to a control prokaryotic host cell that lacks the expression cassette of a).

For example, in one aspect, the present invention provides a host cell comprising: a) an expression cassette comprising a promoter operably linked to a heterologous nucleic acid encoding a major facilitator superfamily (MFS) aromatic acid antiporter; and, and b) an exogenous aromatic substrate of the MFS aromatic acid antiporter. In embodiments, the host cell is capable of increased import of the aromatic substrate of the MFS aromatic acid antiporter into the host cell as compared to a control prokaryotic host cell that lacks the expression cassette of a). As another example, in one aspect, the present invention provides a host cell comprising: a) an expression cassette comprising a promoter operably linked to a heterologous nucleic acid encoding a OMP superfamily porin; and, and b) an exogenous aromatic substrate of the OMP superfamily porin. In embodiments, the host cell is capable of increased import of the aromatic substrate of the OMP superfamily porin into the host cell as compared to a control prokaryotic host cell that lacks the expression cassette of a).

In some embodiments, the aromatic substrate of the transporter is a substrate of a heterologous aromatic prenyltransferase expressed in the host cell. For example, the aromatic substrate of the transporter can be a prenyl acceptor of a heterologous aromatic prenyltransferase expressed in the host cell. In some embodiments, the aromatic substrate of the transporter is an aromatic acid. In some cases, the aromatic substrate of the transporter is olivetolate and/or divarinolic acid. In some cases, the aromatic substrate of the transporter is a decarboxylated derivative of an aromatic acid. In some cases, the substrate of the transporter is olivetol. In some cases, the substrate of the transporter is divarinol. In some cases, the substrate of the transporter is resveratrol, naringenin, or phlorisovalerophenone, or a combination thereof. In some cases, the substrate of the transporter is apigenin, diadzein, genestein, naringenin, olivetol, OA, or resveratrol, or a combination thereof.

In some embodiments, the host cell is a prokaryote. In some cases, the prokaryotic host cell is selected from the group consisting of a prokaryote of the genus Escherichia, Panteoa, Bacillus, Corynebacterium, or Lactococcus. In some embodiments, the cell is Escherichia coli (E. coli), Panteoa citrea, C. glutamicum, Bacillus subtilis, or L. lactis. In some embodiments, the cell is E. coli. In some embodiments, the host cell is a prokaryotic host cell comprising: a) an expression cassette comprising a prokaryotic promoter operably linked to a heterologous nucleic acid encoding a transporter such as a major facilitator superfamily (MFS) aromatic acid antiporter (e.g., pcaK) or an OMP super family porin such as an OprD family porin (e.g., pp3656).

In some embodiments, the host cell is a eukaryote. In some embodiments, the eukaryote is a fungal cell, an insect cell, or a mammalian cell. In some embodiments, the eukaryote is a fungal cell. In some embodiments, the eukaryote is selected from the group consisting of a eukaryote of the genus Saccharomyces, Schizosaccharomyces, Hansela, Kluyveromyces, Yarrowia, Spodoptera, Drosophila, Aedes, Trichoplusia, Estigmene, Bombyx, and Autographica. In some embodiments, the cell is Saccharomyces cerevisiae, or Pichia pastoris. In some embodiments, the cell is Saccharomyces cerevisiae. In some embodiments, the host cell is a eukaryotic host cell comprising: a) an expression cassette comprising a eukaryotic promoter operably linked to a heterologous nucleic acid encoding a major facilitator superfamily (MFS) aromatic acid antiporter or an outer membrane porin (OMP).

In some embodiments, the MFS aromatic acid antiporter is pcaK or a functional fragment thereof. In some embodiments, the MFS aromatic acid antiporter is at least 50%, 55%, 60%, 65%, 70%, 75%, 80%, 85%, 90%, or 95% identical to, or identical to, 50 contiguous amino acids of the sequence set forth in: SEQ ID NO. 6 (MNQAQTNVGKSLDVQSFINQQPLSRYQWRVVLLCFLIVFLDGLDTAAMGFIAPALSQEWGIDR ASLGPVMSAALIGMVFGALGSGPLADRFGRKGVLVGAVLVFGGFSLASAYATNVDQLLVLRFL TGLGLGAGMPNATTLLSEYTPERLKSLLVTSMFCGFNLGMAGGGFISAKMIPAYGWHSLLVIGG VLPLLLALVLMIWLPESARFLVVRNRGTDKVRKTLSPIAPQVVAEAGSFSVPEQKAVAARNVFA VIFSGTYGLGTVLLWLTYFMGLVIVYLLTSWLPTLMRDSGASMEQAAFIGALFQFGGVLSAVGV GWAMDRFNPHKVIGIFYLLAGVFAYAVGQSLGNITLLATLVLVAGMCVNGAQSAMPSLAARFY PTQGRATGVSWMLGIGRFGAILGAWSGATLLGLGWSFEQVLTALLVPAALATVGVVVKGLVSH ADAT). In some embodiments, the MFS aromatic acid antiporter is at least 50%, 55%, 60%, 65%, 70%, 75%, 80%, 85%, 90%, or 95% identical to, or identical to, 100 contiguous amino acids of the sequence set forth in: SEQ ID NO. 6. In some embodiments, the MFS aromatic acid antiporter is at least 50%, 55%, 60%, 65%, 70%, 75%, 80%, 85%, 90%, or 95% identical to, or identical to, 150 contiguous amino acids of the sequence set forth in: SEQ ID NO. 6.

In some embodiments, the MFS aromatic acid antiporter is pcaK or a functional fragment thereof. In some embodiments, the MFS aromatic acid antiporter is at least 50%, 55%, 60%, 65%, 70%, 75%, 80%, 85%, 90%, or 95% identical to, or identical to, 50 contiguous amino acids of the sequence set forth in: SEQ ID NO. 8 (MNQAQTNVGKSLDVQSFINQQPLSRYQWRVVLLCFLIVFLDGLDTAAMGFIAPALSQEWGIDR ASLGPVMSAALIGMVFGALGSGPLADRFGRKGVLVGAVLVFGGFSLASAYATNVDQLLVLRFL TGLGLGAGMPNATTLLSEYTPERLKSLLVTSMFCGFNLGMAGGGFISAKMIPAYGWHSLLVIGG VLPLLLALVLMVWLPESARFLVVRNRGTDKVRKTLSPIAPQVVAEAGSFSVPEQKAVAARNVF AVIFSGTYGLGTVLLWLTYFMGLVIVYLLTSWLPTLMRDSGASMEQAAFIGALFQFGGVLSAVG VGWAMDRFNPHKVIGIFYLLAGVFAYAVGQSLGNITLLATLVLVAGMCVNGAQSAMPSLAARF YPTQGRATGVSWMLGIGRFGAILGAWSGATLLGLGWSFEQVLTALLVPAALATVGVVVKGLVS HADAT). In some embodiments, the MFS aromatic acid antiporter is at least 50%, 55%, 60%, 65%, 70%, 75%, 80%, 85%, 90%, or 95% identical to, or identical to, 100 contiguous amino acids of the sequence set forth in: SEQ ID NO. 8. In some embodiments, the MFS aromatic acid antiporter is at least 50%, 55%, 60%, 65%, 70%, 75%, 80%, 85%, 90%, or 95% identical to, or identical to, 150 contiguous amino acids of the sequence set forth in: SEQ ID NO. 8.

In some embodiments, the OMP is an OprD family porin. In some embodiments, the OprD family porin is pp3656 or a functional fragment thereof. In some embodiments, the OprD family porin is at least 50%, 55%, 60%, 65%, 70%, 75%, 80%, 85%, 90%, or 95% identical to, or identical to, 50 contiguous amino acids of the sequence set forth in: SEQ ID NO. 7 (MSIAFKKTLACSATLLVAPYASAAFVEDFKGSLELRNFYYNRDFRNDGATQSKRDEWAQGFIL NLQSGFTEGPVGFGIDAMGLLGVKLDSSPDRTGSGLLAYDSDRQVEDEYGKFVATAKARMGKT ELRIGGVNPLMPLLWSNNSRLLPQVFRGGSLTVNDIDKLTVTATRINAVKQRNSTDFESLTATGY APVEADHYNYLAFDFKPAKDMTFSLHAAELEDLYKSYFAGIKVIKPLWEGNVIADVRVFDASET GSKKLGEVDNRTLSSYFAYSIKGHTIGGGYQKAWGDTSFAFVNGTDTYLFGESLVSTFTAPEER VWFARYDFDFAALGVPGLLFTTRYMKGDDVNPDLLTSRQAASLRLNGEDGKEWERVTDISYVI QSGPAKGVSFQWRNSTNRSTYADSANENRLIMRYTFNF). In some embodiments, the MFS aromatic acid antiporter is at least 50%, 55%, 60%, 65%, 70%, 75%, 80%, 85%, 90%, or 95% identical to, or identical to, 100 contiguous amino acids of the sequence set forth in: SEQ ID NO. 7. In some embodiments, the MFS aromatic acid antiporter is at least 50%, 55%, 60%, 65%, 70%, 75%, 80%, 85%, 90%, or 95% identical to, or identical to, 150 contiguous amino acids of the sequence set forth in: SEQ ID NO. 7.

embodiments, the OMP is an OprD family porin. In some embodiments, the OprD family porin is pp3656 or a functional fragment thereof. In some embodiments, the OprD family porin is at least 50%, 55%, 60%, 65%, 70%, 75%, 80%, 85%, 90%, or 95% identical to, or identical to, 50 contiguous amino acids of the sequence set forth in: SEQ ID NO. 9 (MSIAFKKTLACSATLLVAPYASAAFVEDFKGSLELRNFYYNRDFRNDGATQSKRDEWAQGFTL NLQSGFTEGPVGFGIDAMGLLGVKLDSSPDRTGSGLLAYDSDRQVEDEYGKFVATAKARMGKT ELRIGGVNPLMPLLWSNNSRLLPQIFRGGSLTVNDIDKLTVTATRVNAVKQRNSTDFESLTATGY APVEADHYNYLAFDFKPAKDMTFSLHAAELEDLYKSYFAGIKVIKPLWEGNVIADVRVFDASET GSKKLGEVDNRTLSSYFAYSIKGHTIGGGYQKAWGDTSFAFVNGTDTYLFGESLVSTFTAPEER VWFARYDFDFAALGVPGLLFTTRYMEGDDVNPDLLTSRQAASLRLNGEDGKEWERVTDISYVI QSGPAKGVSFQWRNSTNRSTYADSANENRLIMRYTFNF). In some embodiments, the MFS aromatic acid antiporter is at least 50%, 55%, 60%, 65%, 70%, 75%, 80%, 85%, 90%, or 95% identical to, or identical to, 100 contiguous amino acids of the sequence set forth in: SEQ ID NO. 9. In some embodiments, the MFS aromatic acid antiporter is at least 50%, 55%, 60%, 65%, 70%, 75%, 80%, 85%, 90%, or 95% identical to, or identical to, 150 contiguous amino acids of the sequence set forth in: SEQ ID NO. 9.

In some embodiments, the (e.g., prokaryotic) host cell further comprises an aromatic prenyltransferase or functional fragment thereof and/or variant thereof, wherein the aromatic prenyltransferase is functional and capable of prenylating the aromatic acid substrate of the transporter (e.g., MFS aromatic acid antiporter or OMP superfamily porin). In some embodiments, the aromatic acid substrate is olivetolate and the aromatic prenyltransferase is functional and capable of prenylating olivetolate. In some cases, the the aromatic prenyltransferase is functional and capable of prenylating olivetolate to produce cannabigerolic acid.

In some embodiments, the aromatic prenyltransferase is CBGAS or NphB or a functional fragment thereof. In some embodiments, the aromatic prenyltransferase is CsPT4 (Lou et al. Nature Feb. 28, 2019), or a functional fragment thereof and/or a variant thereof.

In some embodiments, the aromatic prenyltransferase is a functional fragment of CBGAS. In some embodiments, the CBGAS is at least 50%, 55%, 60%, 65%, 70%, 75%, 80%, 85%, 90%, or 95% identical to, or identical to, 50 contiguous amino acids of the sequence set forth in: SEQ ID NO. 3 (CBGAS; AJN57774.1) MGLSSVCTFSFQTNYHTLLNPHNNNPKTSLLCYRHPKTPIKYSYNNFPSKHCSTKSFHLQNKCSE SLSIAKNSIRAATTNQTEPPESDNHSVATKILNFGKACWKLQRPYTIIAFTSCACGLFGKELLHNT NLISWSLMFKAFFFLVAILCIASFTTTINQIYDLHIDRINKPDLPLASGEISVNTAWIMSIIVALFGLII TIKMKGGPLYIFGYCFGIFGGIVYSVPPFRWKQNPSTAFLLNFLAHIITNFTFYYASRAALGLPFEL RPSFTFLLAFMKSMGSALALIKDASDVEGDTKFGISTLASKYGSRNLTLFCSGIVLLSYVAAILAG IIWPQAFNSNVMLLSHAILAFWLILQTRDFALTNYDPEAGRRFYEFMWKLYYAEYLVYVFI. In some embodiments, the CBGAS is at least 50%, 55%, 60%, 65%, 70%, 75%, 80%, 85%, 90%, or 95% identical to, or identical to, 100 contiguous amino acids of the sequence set forth in: SEQ ID NO. 3. In some embodiments, the CBGAS is at least 50%, 55%, 60%, 65%, 70%, 75%, 80%, 85%, 90%, or 95% identical to, or identical to, 150 contiguous amino acids of the sequence set forth in: SEQ ID NO. 3.

In some cases, the host cell further comprises a (e.g., prokaryotic) promoter operably linked to a nucleic acid encoding an aromatic prenyltransferase such as CBGA synthase (CBGAS). In some embodiments, the CBGAS is at least 50%, 55%, 60%, 65%, 70%, 75%, 80%, 85%, 90%, or 95% identical to, or identical to, 50 contiguous amino acids of the sequence set forth in: SEQ ID NO. 3. In some embodiments, the CBGAS is at least 50%, 55%, 60%, 65%, 70%, 75%, 80%, 85%, 90%, or 95% identical to, or identical to, 100 contiguous amino acids of the sequence set forth in: SEQ ID NO. 3. In some embodiments, the CBGAS is at least 50%, 55%, 60%, 65%, 70%, 75%, 80%, 85%, 90%, or 95% identical to, or identical to, 150 contiguous amino acids of the sequence set forth in: SEQ ID NO. 3.

In some cases the aromatic prenyltransferase (e.g., CBGAS) comprises an N-terminal truncation lacking a plastid or chloroplast retention signal. In some cases the aromatic prenyltransferase (e.g., CBGAS) comprises an N-terminal truncation lacking a plastid retention signal.

In some embodiments, the aromatic prenyltransferase is a functional fragment of NphB. In some embodiments, the NphB is at least 50%, 55%, 60%, 65%, 70%, 75%, 80%, 85%, 90%, or 95% identical to, or identical to, 50 contiguous amino acids of the sequence set forth in: SEQ ID NO.4 (NphB; AFD38743.1) MSEAADVERVYAAMEEAAGLLGVACARDKIYPLLSTFQDTLVEGGSVVVFSMASGRHSTELDF SISVPTSHGDPYATVVEKGLFPATGHPVDDLLADTQKHLPVSMFAIDGEVTGGFKKTYAFFPTD NMPGVAELSAIPSMPPAVAENAELFARYGLDKVQMTSMDYKKRQVNLYFSELSAQTLEAESVL ALVRELGLHVPNELGLKFCKRSFSVYPTLNWETGKIDRLCFAVISNDPTLVPSSDEGDIEKFHNY ATKAPYAYVGEKRTLVYGLTLSPKEEYYKLGAYYHITDVQRGLLKAFDSLED. In some embodiments, the aromatic prenyltransferase is a functional fragment of NphB. In some embodiments, the NphB is at least 50%, 55%, 60%, 65%, 70%, 75%, 80%, 85%, 90%, or 95% identical to, or identical to, 100 contiguous amino acids of the sequence set forth in: SEQ ID NO.4. In some embodiments, the aromatic prenyltransferase is a functional fragment of NphB. In some embodiments, the NphB is at least 50%, 55%, 60%, 65%, 70%, 75%, 80%, 85%, 90%, or 95% identical to, or identical to, 150 contiguous amino acids of the sequence set forth in: SEQ ID NO.4. In some cases, the NphB comprises one or more, or all, of the following mutations: Y288A, Y288N, G286S, A232S, F213H, and/or Y288V. In some cases, the NphB comprises one of the following mutation combinations: Y288N/G286S, Y288A/G286S, Y288A/G286S/A232S, Y288A/G286S/A232S/F213H, Y288V/G286S, Y288V/A232S, or Y288A/A232S. See, Valliere et al. Nature Communications 2019 10:565.

In some cases, the host cell further comprises a (e.g., prokaryotic) promoter operably linked to a nucleic acid encoding an aromatic prenyltransferase such as NphB. In some embodiments, the NphB is at least 50%, 55%, 60%, 65%, 70%, 75%, 80%, 85%, 90%, or 95% identical to, or identical to, 50 contiguous amino acids of the sequence set forth in: SEQ ID NO.4. In some embodiments, the NphB is at least 50%, 55%, 60%, 65%, 70%, 75%, 80%, 85%, 90%, or 95% identical to, or identical to, 100 contiguous amino acids of the sequence set forth in: SEQ ID NO.4. In some embodiments, the NphB is at least 50%, 55%, 60%, 65%, 70%, 75%, 80%, 85%, 90%, or 95% identical to, or identical to, 150 contiguous amino acids of the sequence set forth in: SEQ ID NO.4.

In some embodiments, the host cell comprises an expression cassette comprising a promoter operably linked to a heterologous nucleic acid encoding at least one (e.g., prokaryotic) chaperone.

In some cases, the host cell comprises a cannabinoid synthase. In some cases, the host cell comprises an expression cassette comprising a promoter operably linked to a heterologous nucleic acid encoding the cannabinoid synthase. In some cases the cannabinoid synthase is a CBDAS. In some cases, the cannabinoid synthase is a THCAS.

In some embodiments, the cannabinoid synthase comprises or consists of an amino acid sequence that is at least 50%, 55%, 60%, 65%, 70%, 75%, 80%, 85%, 90%, or 95% identical to, or identical to, 50 contiguous amino acids of the sequence set forth in SEQ ID NO.1 (cannabidiolic-acid synthase; A6P6V9.1; signal peptide removed) NPRENFLKCFSQYIPNNATNLKLVYTQNNPLYMSVLNSTIHNLRFTSDTTPKPLVIVTPSHVSHIQ GTILCSKKVGLQIRTRSGGHDSEGMSYISQVPFVIVDLRNMRSIKIDVHSQTAWVEAGATLGEVY YWVNEKNENLSLAAGYCPTVCAGGHFGGGGYGPLMRNYGLAADNIIDAHLVNVHGKVLDRKS MGEDLFWALRGGGAESFGIIVAWKIRLVAVPKSTMFSVKKIMEIHELVKLVNKWQNIAYKYDK DLLLMTHFITRNITDNQGKNKTAIHTYFSSVFLGGVDSLVDLMNKSFPELGIKKTDCRQLSWIDTI IFYSGVVNYDTDNFNKEILLDRSAGQNGAFKIKLDYVKKPIPESVFVQILEKLYEEDIGAGMYAL YPYGGIMDEISESAIPFPHRAGILYELWYICSWEKQEDNEKHLNWIRNIYNFMTPYVSKNPRLAY LNYRDLDIGINDPKNPNNYTQARIWGEKYFGKNFDRLVKVKTLVDPNNFFRNEQSIPPLPRHRH.

In some embodiments, the cannabinoid synthase comprises or consists of an amino acid sequence that is at least 50%, 55%, 60%, 65%, 70%, 75%, 80%, 85%, 90%, or 95% identical to, or identical to, 100 contiguous amino acids of the sequence set forth in SEQ ID NO.1. In some embodiments, the cannabinoid synthase comprises or consists of an amino acid sequence that is at least 50%, 55%, 60%, 65%, 70%, 75%, 80%, 85%, 90%, or 95% identical to, or identical to, 150 contiguous amino acids of the sequence set forth in SEQ ID NO.1. In some embodiments, the cannabinoid synthase comprises or consists of an amino acid sequence that is at least 50%, 55%, 60%, 65%, 70%, 75%, 80%, 85%, 90%, or 95% identical to, or identical to SEQ ID NO.1.

In some embodiments, the cannabinoid synthase comprises or consists of an amino acid sequence that is at least 50%, 55%, 60%, 65%, 70%, 75%, 80%, 85%, 90%, or 95% identical to, or identical to, 50 contiguous amino acids of the sequence set forth in SEQ ID NO.2 (tetrahydrocannabinolic acid synthase; AB057805.1; secretion signal removed) NPRENFLKCFSKHIPNNVANPKLVYTQHDQLYMSILNSTIQNLRFISDTTPKPLVIVTPSNNSHIQA TILCSKKVGLQIRTRSGGHDAEGMSYISQVPFVVVDLRNMHSIKIDVHSQTAWVEAGATLGEVY YWINEKNENLSFPGGYCPTVGVGGHFSGGGYGALMRNYGLAADNIIDAHLVNVDGKVLDRKS MGEDLFWAIRGGGGENFGIIAAWKIKLVAVPSKSTIFSVKKNMEIHGLVKLFNKWQNIAYKYDK DLVLMTHFITKNITDNHGKNKTTVHGYFSSIFHGGVDSLVDLMNKSFPELGIKKTDCKEFSWIDT TIFYSGVVNFNTANFKKEILLDRSAGKKTAFSIKLDYVKKPIPETAMVKILEKLYEEDVGAGMYV LYPYGGIMEEISESAIPFPHRAGIMYELWYTASWEKQEDNEKHINWVRSVYNFTTPYVSQNPRL AYLNYRDLDLGKTNHASPNNYTQARIWGEKYFGKNFNRLVKVKTKVDPNNFFRNEQSIPPLPPH HH.

In some embodiments, the cannabinoid synthase comprises or consists of an amino acid sequence that is at least 50%, 55%, 60%, 65%, 70%, 75%, 80%, 85%, 90%, or 95% identical to, or identical to, 100 contiguous amino acids of the sequence set forth in SEQ ID NO.2. In some embodiments, the cannabinoid synthase comprises or consists of an amino acid sequence that is at least 50%, 55%, 60%, 65%, 70%, 75%, 80%, 85%, 90%, or 95% identical to, or identical to, 150 contiguous amino acids of the sequence set forth in SEQ ID NO.2. In some embodiments, the cannabinoid synthase comprises or consists of an amino acid sequence that is at least 50%, 55%, 60%, 65%, 70%, 75%, 80%, 85%, 90%, or 95% identical to, or identical to SEQ ID NO.2.

In some embodiments, the cannabinoid synthase comprises or consists of an amino acid sequence at least 80%, 85%, 90%, 95%, or 99% identical to 150 contiguous amino acids of SEQ ID NO.1 or SEQ ID NO.2. In some embodiments, the cannabinoid synthase comprises or consists of an amino acid sequence at least 50% or 55% identical to 300 contiguous amino acids of SEQ ID NO.1 or SEQ ID NO.2. In some embodiments, the cannabinoid synthase comprises or consists of an amino acid sequence at least 80%, 85%, 90%, 95%, or 99% identical to 300, or all, contiguous amino acids of SEQ ID NO.1 or SEQ ID NO.2. In some embodiments, the cannabinoid synthase is a Cannabis sativa cannabinoid synthase.

In some embodiments, the cannabinoid synthase comprises or consists of an amino acid sequence at least 80%, 85%, 90%, 95%, or 99% identical to 150 contiguous amino acids of SEQ ID NO.3. In some embodiments, the cannabinoid synthase comprises or consists of an amino acid sequence at least 50% or 55% identical to 300 contiguous amino acids of SEQ ID NO.3. In some embodiments, the cannabinoid synthase comprises or consists of an amino acid sequence at least 80%, 85%, 90%, 95%, or 99% identical to 300, or all, contiguous amino acids of SEQ ID NO.3. In some embodiments, the host cell comprises a nucleic acid encoding CBGA synthase and a nucleic acid encoding a cannabinoid synthase selected from the group consisting of THCA synthase and CBDA synthase, or a combination of one or more nucleic acids encoding two or all thereof. In some cases, the host cell comprising the CBGA synthase expression cassette further comprises a nucleic acid encoding a THCA synthase and/or CBDA synthase, each synthase independently operably linked to a promoter in the same or a different expression cassette.

In some cases, the host cell comprising the expression cassette comprising a heterologous nucleic acid encoding the transporter (e.g., MFS aromatic acid antiporter such as pcaK or OMP superfamily porin such as an OprD family porin, such as pp3656) further comprises a nucleic acid encoding an aromatic prenyltransferase, a THCA synthase and/or CBDA synthase, each synthase and/or prenyltransferase independently operably linked to a promoter in the same or a different expression cassette. In some cases, the host cell comprising the expression cassette comprising a heterologous nucleic acid encoding the transporter (e.g., MFS aromatic acid antiporter such as pcaK or OMP superfamily porin such as an OprD family porin, such as pp3656) further comprises a nucleic acid encoding an aromatic prenyltransferase independently operably linked to a promoter in the same or a different expression cassette. In some cases, the host cell comprising the expression cassette comprising a heterologous nucleic acid encoding the transporter (e.g., MFS aromatic acid antiporter such as pcaK or OMP superfamily porin such as an OprD family porin, such as pp3656) further comprises a nucleic acid encoding an aromatic prenyltransferase and CBDA synthase, each synthase and prenyltransferase independently operably linked to a promoter in the same or a different expression cassette.

In some embodiments, the cannabinoid synthase, or at least one encoded cannabinoid synthase, is a truncated cannabinoid synthase selected from the group consisting of a truncated THCA synthase and a truncated CBDA synthase, wherein the truncation is a deletion of all or part of a signal peptide, a plastid retention signal, and/or a chloroplast retention signal. In some embodiments, the cannabinoid synthase comprises a deletion of all or part of a transmembrane or membrane-associated region, such that the cannabinoid synthase is not membrane-associated, or would not be membrane-associated if expressed in a eukaryotic system.

In some embodiments, the promoter operably linked to the nucleic acid encoding the transporter is a constitutive promoter. In some embodiments, the promoter operably linked to the nucleic acid encoding the transporter is an inducible promoter. In some cases, the promoter operably linked to the nucleic acid encoding the aromatic prenyltransferase is a constitutive promoter. In some embodiments, the promoter operably linked to the nucleic acid encoding the aromatic prenyltransferase is an inducible promoter. In some cases, the promoter operably linked to the nucleic acid encoding the transporter is a constitutive promoter and the promoter operably linked to the nucleic acid encoding the aromatic prenyltransferase is a constitutive promoter. In some cases, the promoter operably linked to the nucleic acid encoding the transporter is an inducible promoter and the promoter operably linked to the nucleic acid encoding the aromatic prenyltransferase is an inducible promoter. In some cases, the promoter operably linked to the nucleic acid encoding the aromatic prenyltransferase and the promoter operably linked to the nucleic acid encoding the transporter is the same promoter. In some cases, the promoter operably linked to the nucleic acid encoding the aromatic prenyltransferase and the promoter operably linked to the nucleic acid encoding the transporter are two different promoters.

In some embodiments, where the host cell comprises two or more expression cassettes comprising different cannabinoid synthases, each expression cassette comprises an inducible promoter operably linked to a cannabinoid synthase. In some embodiments, where the host cell comprises two or more expression cassettes comprising different cannabinoid synthases, at least one expression cassette comprises an inducible promoter operably linked to a cannabinoid synthase. In some embodiments, where the host cell comprises two or more expression cassettes comprising different cannabinoid synthases, at least one expression cassette comprises a constitutive promoter operably linked to a cannabinoid synthase.

In some embodiments, the promoter operably linked to the nucleic acid encoding the cannabinoid synthase is a constitutive promoter. In some embodiments, the promoter operably linked to the nucleic acid encoding the cannabinoid synthase is an inducible promoter. In some embodiments, where the host cell comprises two or more expression cassettes comprising different cannabinoid synthases, each expression cassette comprises a constitutive promoter operably linked to a cannabinoid synthase.

In some embodiments, where the host cell comprises two or more expression cassettes comprising different cannabinoid synthases, each expression cassette comprises an inducible promoter operably linked to a cannabinoid synthase. In some embodiments, where the host cell comprises two or more expression cassettes comprising different cannabinoid synthases, at least one expression cassette comprises an inducible promoter operably linked to a cannabinoid synthase. In some embodiments, where the host cell comprises two or more expression cassettes comprising different cannabinoid synthases, at least one expression cassette comprises a constitutive promoter operably linked to a cannabinoid synthase.

In some embodiments, the host cell comprises or further comprises an expression cassette comprising a promoter operably linked to a nucleic acid encoding one or more MEP pathway enzymes selected from the group consisting of dxs, ispC, ispD, ispE, ispF, ispG, ispH, and idi. In some cases, the host cell comprises or further comprises an expression cassette comprising a promoter operably linked to a nucleic acid encoding the bifunctional MEP pathway enzyme ispDF. In some cases, the expression cassette comprising the bifunctional ispDF enzyme further comprises the one or more MEP pathway enzymes selected from the group consisting of dxs, ispC, ispD, ispE, ispF, ispG, ispH, and idi. In some cases, the expression cassette comprising the bifunctional ispDF enzyme further comprises dxs and idi.

In some cases, the host cell comprises a higher level of expression of one or more MEP pathway genes as compared to a control cell that does not comprise the expression cassette comprising the bifunctional ispDF enzyme. In some cases, the host cell comprises a higher level of expression of dxs and idi as compared to a control cell that does not comprise the expression cassette comprising the bifunctional ispDF enzyme.

In some embodiments, the host cell comprises, or further comprises, an expression cassette comprising a promoter operably linked to a nucleic acid encoding an ispDE bifunctional MEP pathway enzyme. In some embodiments, the bifunctional MEP pathway enzyme comprises a flexible linker peptide between an ispD domain and an ispE domain. In some embodiments, the flexible linker comprises the sequence of SLGGGGSAAA. In some cases, the linker sequence has a greater than 65% random coil formation as determined by GOR algorithm, version IV (Methods in Enzymology 1996 R. F. Doolittle Ed., vol 266, 540-553).

In some embodiments, the ispDE bifunctional MEP pathway enzyme comprises or consists of an amino acid sequence that is at least 50%, 55%, 60%, 65%, 70%, 75%, 80%, 85%, 90%, or 95% identical to, or identical to, 50 contiguous amino acids of the sequence set forth in SEQ ID NO.10 (MATTHLDVCAVVPAAGFGRRMQTECPKQYLSIGNQTILEHSVHALLAHPRVKRVVIAISPGDSR FAQLPLANHPQITVVDGGDERADSVLAGLKAAGDAQWVLVHDAARPCLHQDDLARLLALSET SRTGGILAAPVRDTMKRAEPGKNAIAHTVDRNGLWHALTPQFFPRELLHDCLTRALNEGATITD EASALEYCGFHPQLVEGRADNIKVTRPEDLALAEFYLTRTIHQENTSLGGGGSAAAMRTQWPSP AKLNLFLYITGQRADGYHTLQTLFQFLDYGDTISIELRDDGDIRLLTPVEGVEHEDNLIVRAARLL MKTAADSGRLPTGSGANISIDKRLPMGGGLGGGSSNAATVLVALNHLWQCGLSMDELAEMGL TLGADVPVFVRGHAAFAEGVGEILTPVDPPEKWYLVAHPGVSIPTPVIFKDPELPRNTPKRSIETL LKCEFSNDCEVIARKRFREVDAVLSWLLEYAPSRLTGTGACVFAEFDTESEARQVLEQAPEWLN GFVAKGANLSPLHRAML).

In some cases, the host cell comprises or further comprises an expression cassette comprising a promoter operably linked to a nucleic acid encoding the bifunctional MEP pathway enzyme ispDE. In some cases, the expression cassette comprising the bifunctional ispDE enzyme further comprises one or more MEP pathway enzymes selected from the group consisting of dxs, ispC, ispF, ispG, ispH, and idi. In some cases, the expression cassette comprising the bifunctional ispDE enzyme further comprises dxs, ispF and idi. In some cases, the expression cassette comprising the bifunctional ispDE enzyme further comprises a bifunctional ispDF enzyme (see PCT/CA2018/051074). In some cases, the expression cassette comprising the bifunctional ispDE enzyme further comprises one or more MEP pathway enzymes selected from the group consisting of dxs, ispC, ispDF, ispG, ispH, and idi.

In some cases, the host cell comprises a higher level of expression of one or more MEP pathway genes as compared to a control cell that does not comprise the expression cassette comprising the bifunctional ispDE enzyme. In some cases, the host cell comprises a higher level of expression of dxs and idi as compared to a control cell that does not comprise the expression cassette comprising the bifunctional ispDE enzyme. In some cases, the host cell comprises a higher level of expression of one or more MEP pathway genes as compared to a control cell that does not comprise the expression cassette comprising the bifunctional ispDE enzyme. In some cases, the host cell comprises a higher level of expression of dxs and idi as compared to a control cell that does not comprise the expression cassette comprising the bifunctional ispDE enzyme.

In some embodiments, the host cell comprises an expression cassette comprising a promoter operably linked to a nucleic acid encoding GPP synthase.

In some embodiments, the host cell is in a culture medium that comprises the substrate (e.g., olivetolate (OA) of the transporter (e.g., MFS aromatic acid antiporter or OMP superfamily porin such as an OprD family porin, such as pp3656). In some cases, the substrate (e.g., olivetolate (OA) is exogenous to the host cell. For example, the substrate (e.g., OA) can be exogenously supplied to a culture media in which the host cell is cultured.

In some embodiments, the host cell comprises a deletion in 1, 2, 3, 4, 5, 6, 7, 8, or all of the genes selected from the group consisting of ackA-pta, poxB, ldhA, dld, adhE, pps, and atoDA.

In some embodiments, the host cell comprises a PDH bypass. See, e.g., Valliere et al. 2019. In some embodiments, the PDH bypass comprises heterologously expressed pyruvate oxidase and acetyl-phosphate transferase.

In embodiments, one or more, or two or more, or all, expression cassettes are integrated into the genome of the host cell. In additional or alternative embodiments, one or more expression cassettes are not integrated into the genome of the host cell.

In a second aspect, the present invention provides a method of increasing the transport of an aromatic substrate of an MFS aromatic acid antiporter into a (e.g., prokaryotic) host cell. In some embodiments, the method comprises culturing a host cell described herein in culture media containing the aromatic substrate under conditions suitable to express the transporter or a functional fragment thereof.

In another aspect, the present invention provides a method of prenylating a substrate (e.g., olivetolate (OA) of a transporter (e.g., MFS aromatic acid antiporter or OMP superfamily porin such as an OprD family porin, such as pp3656). In some embodiments, the method comprises culturing a host cell described herein in culture media containing the aromatic substrate of the transporter and the aromatic prenyltransferase, thereby prenylating the aromatic substrate of the transporter. In some embodiments, the substrate is olivetolate. In some embodiments, the aromatic prenyltransferase is functional and capable of transferring a geranyl moiety (e.g., from a geranyl-diphosphate) to the aromatic substrate. In some embodiments, the aromatic prenyltransferase is functional and capable of transferring a farnesyl moiety (e.g., from a farnesyl-diphosphate) to the aromatic substrate. In some embodiments, the aromatic prenyltransferase is functional and capable of transferring a neryl moiety (e.g., from a neryl-diphosphate) to the aromatic substrate. In some embodiments, the aromatic prenyltransferase is functional and capable of transferring a geranyl moiety (e.g., from a geranyl-diphosphate) and/or a neryl moiety (e.g., from a neryl-diphosphate) to the aromatic substrate. In some embodiments, the aromatic prenyltransferase is functional and capable of transferring a geranyl moiety (e.g., from a geranyl-diphosphate), a farnesyl moiety (e.g., from a farnesyl-diphosphate), and/or a neryl moiety (e.g., from a neryl-diphosphate) to the aromatic substrate.

In some embodiments, the aromatic prenyltransferase has geranyl-diphosphate:olivetolate geranyltransferase activity. In some embodiments, the aromatic prenyltransferase is a CBGA synthase, an orthologue thereof, or a functional fragment thereof. In some embodiments, the aromatic prenyltransferase is a CBGA synthase having the sequence of SEQ ID NO.3 or a functional fragment thereof. In some embodiments, the aromatic prenyltransferase is NphB, an orthologue thereof, or a functional fragment thereof. In some embodiments, the aromatic prenyltransferase is NphB having the sequence of SEQ ID NO.4, or a functional fragment thereof. In some embodiments, the aromatic acid is olivetolate and the aromatic prenyltransferase is a CBGA synthase or NphB and the method comprises producing cannabigerolic acid.

In some embodiments, the method increases the production of a prenylated product of the aromatic prenyltransferase and the aromatic acid substrate as compared to a control method performed under conditions that do not express, or express a lower amount or activity of, the transporter. In some embodiments, the method increases the production of a prenylated olivetolate product as compared to a control method performed under conditions that do not express, or express a lower amount or activity of, the transporter.

In some embodiments, the method comprises culturing a prokaryotic host cell described herein in a suitable culture medium under conditions suitable to induce expression in one or more host cell expression cassettes, and then harvesting the cultured cells or spent medium, thereby obtaining the target metabolic product. In some embodiments, the target metabolic product is THCA, CBDA, CBCA, CBGA, CBN, CBC, THC, or CBD, or a mixture of one or more thereof. In some embodiments, the culture medium comprises exogenous olivetolate. In some embodiments, the culture medium comprises exogenous DVA. In some embodiments, the method comprises adding olivetolate to the culture medium and/or providing a culture medium comprising olivetolate and culturing the host cell in the provided culture medium. In some embodiments, the method comprises adding DVA to the culture medium and/or providing a culture medium comprising DVA and culturing the host cell in the provided culture medium.

In some embodiments, the method comprises harvesting and lysing the cultured cell, thereby producing cell lysate. In some embodiments, the method comprises purifying a target cannabinoid from the cell lysate, thereby producing a purified target cannabinoid. In some embodiments, the method comprises purifying the target cannabinoid from the spent culture medium, thereby producing a purified target cannabinoid.

In some embodiments, the purified target metabolic product is a cannabinoid and the method comprises formulating the cannabinoid in a pharmaceutical composition. In some embodiments, the purified target metabolic product is a cannabinoid and the method comprises forming a salt, prodrug, or solvate of the purified cannabinoid. In some embodiments, the purified target metabolic product is a cannabinoid and the method comprises forming a decarboxylate of the purified cannabinoid. In some embodiments, the decarboxylate is formed by heating the purified target metabolic product. In some embodiments, the method comprises heating the host cells, host cell lysate, or spent culture medium to decarboxylate the target metabolic product.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1 illustrates a schematic of a cannabinoid pathway for production of one or more cannabinoids selected from the group consisting of CBGA, CBGVA, THCA, CBDA, CBCA, THCVA, CBCVA, CBDVA, CBN, THC, CBD, CBC, THCV, CBCV, and CBDV.

FIG. 2 illustrates a pcaK (left) and pp3656 (right) expression plasmid, wherein expression of the pcaK or pp3656 transgene is under the control of an arabinose promoter.

FIG. 3 illustrates a B5 expression plasmid construct. The B5 plasmid expresses IspDF1 chimera, idi, and dxs for the non-mevalonate (MEP) pathway, expresses GPP synthase for production of GPP, and expresses an optimized NphB variant aromatic prenyltransferase for production of CBGA from OA and GPP.

FIG. 4 illustrates SDS-PAGE analysis of an expression culture of E. coli harboring an NphB expression plasmid and a: pcaK expression plasmid (B5-pcaK); a pp3656 expression plasmid (B5-3656); or a control expression plasmid (B5-pBAD). pcaK expected size 47.1 kDA; pp3656 expected size: 46.7 kDa; NphB expected size 33.7 kDA.

FIG. 5 illustrates a comparison of the olivetolate permeability in the presence and absence of aromatic transporters.

FIG. 6 illustrates a comparison of the olivetolate cell permeability at different temp in the presence of aromatic transporter, pcaK.

FIG. 7 illustrates olivetolate cell permeability in presence of aromatic transporter pcaK at different incubation times.

FIG. 8 illustrates increased olivetolate uptake inside cells expressing pcaK or pp3656 as compared to a control cell not expressing a heterologous transporter. Increased OA uptake inside the cell was detected over 24 to 48 hours after expression and induction of pBAD-pcaK and pBAD-3656 compared to BL21 control without expression of additional transporters.

FIG. 9 illustrates increased production of CBGA in cells expressing NphB and either pcaK or pp3656 as compared to a control cell expressing an NphB variant optimized for olivetolate prenylation (see, Valliere et al. Nature Communications 2019 10:565) but not expressing a heterologous transporter.

FIG. 10 illustrates expression constructs encoding a non-mevalonate pathway for production of IPP and DMAPP

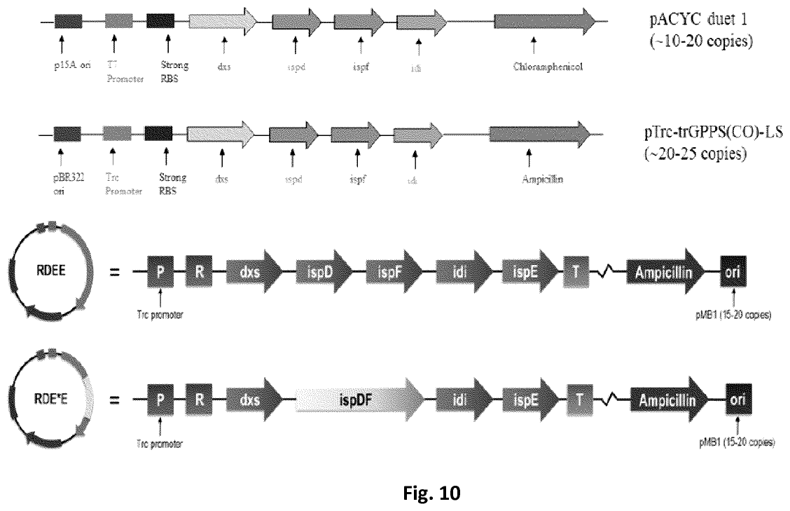

FIG. 11 illustrates expression constructs encoding an aromatic prenyltransferase enzyme; a CBGAS enzyme.

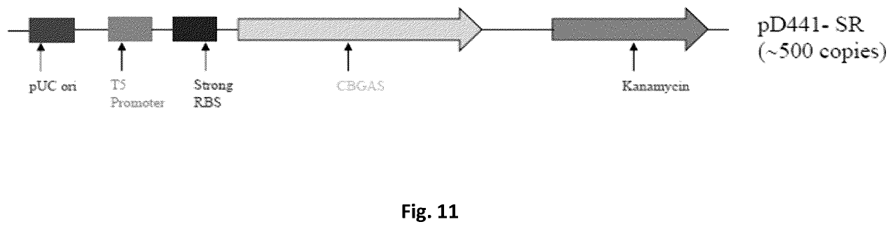

FIG. 12 illustrates expression constructs encoding the aromatic prenyltransferase enzyme NphB.

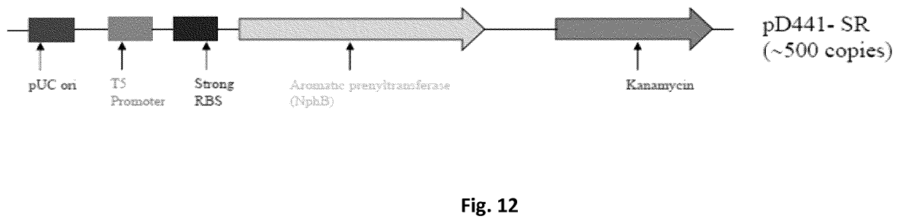

FIG. 13 illustrates an expression construct encoding a THCAS enzyme.

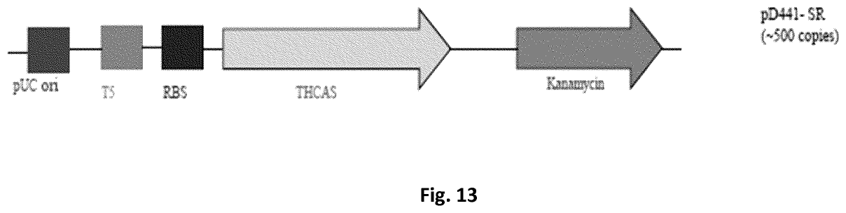

FIG. 14 illustrates expression of novel IspDFs in E. coli as shown by SD S-PAGE analysis. Lanes 1 and 5: total and purified IspDF₁ extract respectively, lanes 2 and 6: total and purified IspDF₂ extract respectively, lanes 4 and 7: total and purified IspDF₃ extract respectively, lanes 3 and 8: protein ladder.

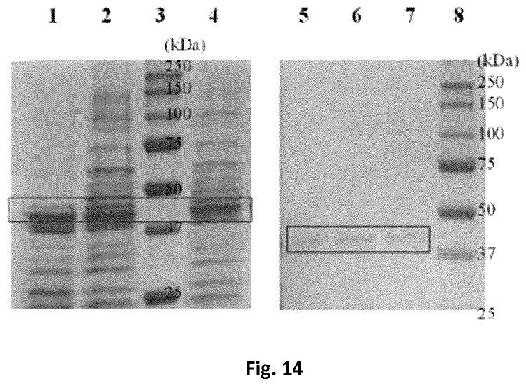

FIG. 15 illustrates a protein sequence alignment of various IspDF fusion proteins.

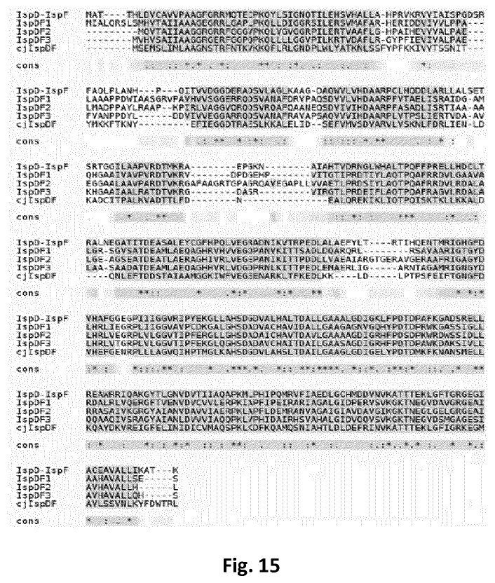

FIG. 16 illustrates an SDS/PAGE image of soluble protein fraction of pSASDFI. Lane 1: E. coli BL21(DE3), lane 2: protein ladder, lane 3 and 4: SASDFI. The bands corresponding to protein are: Dxs (band a, 68.2 kDa), IspD (band b, 25.7 kDa), IspF (band d, 16.9 kDa) and Idi (band c, 21.2 kDa).

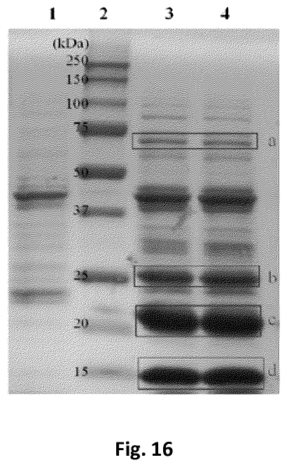

FIGS. 17 (a)-(b) illustrates influence of rate-limiting steps on MEP pathway flux. (a) Lycopene production, (b) Isoprene production. The IPTG concentrations used for induction are denoted in the legends. Primary Y-axis is terpene titer and secondary Y-axis is normalized terpene titer.

FIGS. 18 (a)-(b) Influence of novel IspDF fusions on MEP pathway flux. (a) Lycopene production, (b) Isoprene production. The IPTG concentrations used for induction are denoted in the legends. Primary Y-axis is terpene titer and secondary Y-axis is normalized terpene titer.

FIGS. 19 (a)-(d) illustrate homology models for the fusion proteins generated by SWISS-MODEL tool. (a) cjIspDF (Liu et al. Biosci Rep. 2018 Feb. 28; 38(1): BSR20171370), (b) IspDF₁, (c) IspDF₂ and (d) IspDF₃. The IspD domain is in pink, the IspF domain is in blue and linker is in green. The N-terminal residue is colored black and C-terminal residue is colored orange.

FIG. 20 illustrates effect of IspE overexpression on lycopene production. The IPTG concentrations used for induction are from left to right 0 μM, 25 μM, and 50 μM for each construct. Primary Y-axis is terpene titer and secondary Y-axis is normalized terpene titer.

FIG. 21(a)-(b) illustrates linkers for IspDF₁ and their effect on MEP pathway flux. (a) Strains overexpressing Dxs, IspDF chimeras and Idi, (b) strains overexpressing Dxs, IspDF chimeras, IspE and Idi. The IPTG concentrations used for induction are from left to right 0 μM, 25 μM, and 50 μM for each construct. Primary Y-axis is terpene titer and secondary Y-axis is normalized terpene titer.

FIG. 22(a)-(b) illustrates linkers for non-natural fusions of E. coli IspD and IspF; and their effect on MEP pathway flux. (a) Strains overexpressing Dxs, IspDF chimeras and Idi, (b) strains overexpressing Dxs, IspDF chimeras, IspE and Idi. The IPTG concentrations used for induction are from left to right 0 μM, 25 μM, and 50 μM for each construct. Primary Y-axis is terpene titer and secondary Y-axis is normalized terpene titer.

FIG. 23 illustrates linkers for non-natural fusions of E. coli IspD and IspF on MEP pathway flux. The IPTG concentrations used for induction are from left to right 0 μM, 25 μM, and 50 μM for each construct. Primary Y-axis is terpene titer and secondary Y-axis is normalized terpene titer.

FIG. 24 illustrates effect of domain separation of IspDF₁ on MEP pathway flux. The IPTG concentrations used for induction are from left to right 0 μM, 25 μM, and 50 μM for each construct. Primary Y-axis is terpene titer and secondary Y-axis is normalized terpene titer.

FIG. 25 illustrates non-natural fusions of IspE and their effect on MEP pathway flux. The IPTG concentrations used for induction are from left to right 0 μM, 25 μM, and 50 μM for each construct. Primary Y-axis is terpene titer and secondary Y-axis is normalized terpene titer.

FIG. 26 illustrates a comparison plot showing lycopene production in the indicated ispDE overexpression strains as compared to different control constructs. Titer (left) and normalized titer (right) values are provided. The blank places denoted by ‘-’.

### DETAILED DESCRIPTION OF THE INVENTION

Described herein is a host cell genetic engineering strategy for increasing the transport of an aromatic acid into a prokaryotic host cell. The aromatic acid can then be provided intracellularly as a substrate for one or more down-stream enzymatic steps to produce a desired target metabolite. For example, the aromatic acid can be a substrate of a heterologous aromatic prenyltransferase enzyme. The aromatic prenyltransferase can prenylate the aromatic acid to produce a prenylated product. The prenyl donor can be an endogenous prenyl donor or a heterologous prenyl donor. In certain embodiments, the prenyl donor is geranyl-diphosphate. In some embodiments, the prenyl donor is nerylpyrophosphate. In some embodiments, the prenyl donor is an organic pyrophosphate. In some embodiments, the prenyl donor is an organic pyrophosphate naturally occurring in Cannabis sativa. In some embodiments, the prenyl donor is an organic pyrophosphate naturally occurring in E. coli. In some embodiments, the prenyl donor is an organic pyrophosphate selected from the group consisting of isopentyl diphosphate (IPP), dimethylallyl diphosphate (DMAPP), geranyl diphosphate (GPP), farnesyl diphosphate (FPP), geranyl-geranyl diphosphate (GGPP), and their isomers, such as the isomer of GPP neryl-diphosphate.

In some cases, the prenyl donor is produced partially or entirely, or an increased amount of prenyl donor is provided, by a heterologous expression cassette comprising a nucleic acid encoding a GPP synthase. In some cases, the prenyl donor is produced partially or entirely, or an increased amount of prenyl donor is provided, by a heterologous expression cassette comprising a nucleic acid encoding a component of a non-mevalonate pathway. In some cases, the prenyl donor is produced partially or entirely, or an increased amount of prenyl donor is provided, by a heterologous expression cassette comprising a nucleic acid encoding a bifunctional ispDF enzyme. In some cases, the prenyl donor is produced partially or entirely, or an increased amount of prenyl donor is provided, by a heterologous expression cassette comprising a nucleic acid encoding a bifunctional ispDE enzyme.

In embodiments where the substrate of the heterologous transporter is a substrate of a heterologous aromatic prenyltransferase enzyme expressed in the host cell, the substrate is typically a prenyl acceptor. For example, the prenyl acceptor can be olivetolate or DVA. Thus, in some embodiments, methods and compositions are described herein for producing a prenylated olivetolate product. Additionally or alternatively, methods and compositions are described herein for producing a prenylated divarinic acid product. In embodiments where the prenyl donor is geranylpyrophosphate and the prenyl acceptor is olivetolate, the prenylated product can be cannabigerolic acid (CBGA). In embodiments where the prenyl donor is nerylpyrophosphate and the prenyl acceptor is olivetolate, the prenylated product can be cannabinerolate (CBNRA). In some embodiments, the prenyl acceptor is divarinic acid (DVA). Thus in some embodiments, methods and compositions are described herein for producing a prenylated divarinic acid product. In embodiments where the prenyl donor is geranylpyrophosphate and the prenyl acceptor is DVA, the prenylated product can be cannabigerovarinic acid acid (CBGVA). In some embodiments, the prenyl donor is nerylpyrophosphate, the prenyl acceptor is olivetolate, the prenylated product is CBNRA, and the aromatic prenyl transferase is NphB, or a functional fragment thereof.

Prenylated aromatic products (e.g., prenylated aromatic acids) such as prenylated olivetolate, a downstream enzymatic product thereof, or a decarboxylate thereof, can be isolated as a target metabolite from the host cell, a lysate thereof, or a spent culture media thereof. In some cases, the isolated target metabolite, a salt thereof, a solvate thereof, a derivative thereof, and/or a decarboxylate thereof, can be used as a drug active ingredient in a pharmaceutical formulation.

Accordingly, in embodiments where the prenylated aromatic product is prenylated olivetolate, olivetol, DVA, or divarinol, the methods and compositions described herein can be used in the production of cannabinoids in a host cell. For example, the host cell can co-express a heterologous cannabinoid synthase such as CBDA synthase. Similarly, in some embodiments, the methods and compositions described herein can be used in the production of cannabinoid precursors in the host cell, wherein the precursors are isolated and used as reactants in one or more in vitro reactions to produce a target product such as a cannabinoid or derivative thereof.

These in vitro reactions can comprise a synthetic chemical scheme to produce a target product such as a cannabinoid or derivative thereof. These in vitro reactions can additionally or alternatively comprise one or more enzyme-catalyzed in vitro reactions. For example, the cannabinoid precursor can be contact with a cannabinoid synthase isolated from a host cell, or in a host cell lysate. As yet another alternative, the cannabinoid precursors can be isolated and used as an input to a second microbial synthesis step using a different prokaryotic host or eukaryotic host that heterologously expresses a cannabinoid synthase.

Also described herein are methods and compositions for co-expression of the heterologous transporter, the aromatic prenyl transferase functional and capable of prenylating a substrate of the heterologous transporter, and one or more additional pathway components. As described herein, the one or more additional pathway components can include a cannabinoid synthase (e.g., THCAS and/or CBDAS) and one or more helper pathway components to thereby produce detectable quantities of a cannabinoid in the (e.g., prokaryotic) host cell system. Another exemplary helper pathway component is a mevalonate-independent (MEP) pathway component, such as a bifunctional ispDF enzyme. Another exemplary helper pathway component is a mevalonate-independent (MEP) pathway component, such as a bifunctional ispDE enzyme. Another exemplary helper pathway component is GPP synthase. Expression of one or more components of one or more helper pathways can be used to produce the target cannabinoid. Expression of nucleic acids encoding the heterologous transporter, the aromatic prenyl transferase, the cannabinoid synthase(s), one or more of the one or more helper pathway component(s), and combinations thereof can be controlled by one or more heterologous promoters.

In some embodiments, the cannabinoid synthase is THCAS. In some embodiments, the cannabinoid synthase is CBDAS. In some embodiments, the prokaryotic host cell comprises an expression cassette comprising a promoter operably linked to THCAS and an expression cassette comprising a promoter operably linked to CBDAS.

### Definitions

“THCAS” or “tetrahydrocannabinolic acid synthase” refers to an enzyme that catalyzes conversion of cannabigerolic acid to tetrahydrocannabinolic acid.

“CBDAS” or “cannabidiolic acid synthase” refers to an enzyme that catalyzes conversion of cannabigerolic acid to cannabidiolic acid.

“CBGAS” or “cannabigerolic acid synthase” refers to an enzyme that catalyzes conversion of olivetolate and GPP to cannabigerolic acid.

The following abbreviations are used herein: “G3P” means glyceraldehyde 3-phosphate; “DOXP” means 1-Deoxy-D-xylulose 5-phosphate; “MEP” means 2-C-methylerythritol 4-phosphate; “CDP-ME” means 4-diphosphocytidyl-2-C-methylerythritol; “CDP-MEP” means 4-diphosphocytidyl-2-C-methyl-D-erythritol 2-phosphate; “MECPP” means 2-C-methyl-D-erythritol 2,4-cyclodiphosphate; “HMBPP” means (E)-4-Hydroxy-3-methyl-but-2-enyl pyrophosphate; “IPP” means isopentenyl disphosphate; “DMAPP” means dimethylallyl diphosphate; “GPP” means geranyl pyrophosphate.

“DXP pathway” and “MEP pathway” refer to the non-mevalonate pathway, also known as the mevalonate-independent pathway. The genes of the MEP pathway are dxs, ispC, ispD, ispE, ispF, ispG, ispH, and idi.

“dxs” refers to DOXP synthase; “ispC” refers to DOXP reductase; “ispD” refers to 2-C-methyl-D-erythritol 4-phosphate cytidylyltransferase; “ispE” refers to 4-diphosphocytidyl-2-C-methyl-D-erythritol kinase; “ispF” refers to 2-C-methyl-D-erythritol 2,4-cyclodiphosphate synthase; “ispG” refers to HMB-PP synthase; “ispH” refers to HMB-PP reductase; “idi” refers to isopentenyl/dimethylallyl diphosphate isomerase; “ispA” refers to farnesyl diphosphate synthase, also known as “GPP synthase,” which can convert DMAPP+IPP to GPP and GPP+IPP to farnesyl pyrophosphate.

The term “ispDF” refers to a bifunctional single-chain enzyme having two different active sites and exhibiting ispD activity (EC 2.7.7.60) and ispF activity (EC 4.6.1.12). Typically, ispDF is a naturally occurring bifunctional enzyme or a derivative of a naturally occurring bifunctional enzyme having one or more modifications such as a deletion, insertion, or substitution of one or more amino acids.

“OA” refers to olivetolate; “CBGA” refers to cannabigerolic acid; “CBNRA” refers to cannabinerolic acid; “CBNA” refers to cannabinolic acid; “cannabinol” or “CBN” refers to 6,6,9-trimethyl-3-pentylbenzo[c]chromen-1-ol; “CBGVA” refers to cannabigerivarinic acid; “THCA” refers to tetrahydrocannabinolic acid, including the Δ⁹ isomer; “CBDV” refers to cannabidivarin; “CBC” refers to cannabichromene; “CBCA” refers to cannabichromenic acid; “CBCV” refers to cannabichromevarin; “CBG refers to cannabigerol; “CBGV” refers to cannabigerovarin; “CBE” refers to cannabielsoin; “CBL” refers to cannabicyclol; “CBV” refers to cannabivarin; “CBT” refers to cannabitriol; “THCV” refers to tetrahydrocannibivarin (THCV); “THC” refers to tetrahydrocannabinol, and “Δ⁹-THC” refers to Δ⁹-tetrahydrocannabinol; “CBDA” refers to cannabidiolic acid.

As used herein, the terms “cannabidiol,” “CBD,” or “cannabidiols” refer to one or more of the following compounds, and, unless a particular other stereoisomer or stereoisomers are specified, includes the compound “Δ²-cannabidiol.” These compounds are: (1) g-cannabidiol (2-(6-isopropenyl-3-methyl-5-cyclohexen-1-yl)-5-pentyl-1,3-benzenediol); (2) g-cannabidiol (2-(6-isopropenyl-3-methyl-4-cyclohexen-1-yl)-5-pentyl-1,3-benzenediol); (3) g-cannabidiol (2-(6-isopropenyl-3-methyl-3-cyclohexen-1-yl)-5-pentyl-1,3-benzenediol); (4) Δ^{3,7}-cannabidiol (2-(6-isopropenyl-3-methylenecyclohex-1-yl)-5-pentyl-1,3-benzenediol); (5) Δ²-cannabidiol (2-(6-isopropenyl-3-methyl-2-cyclohexen-1-yl)-5-pentyl-1,3-benzenediol); (6) Δ¹-cannabidiol (2-(6-isopropenyl-3-methyl-1-cyclohexen-1-yl)-5-pentyl-1,3-benzenediol); and (7) g-cannabidiol (2-(6-isopropenyl-3-methyl-6-cyclohexen-1-yl)-5-pentyl-1,3-benzenediol).

These compounds have one or more chiral centers and two or more stereoisomers as stated below: (1) (1) Δ⁵-cannabidiol has 2 chiral centers and 4 stereoisomers; (2) g-cannabidiol has 3 chiral centers and 8 stereoisomers; (3) g-cannabidiol has 2 chiral centers and 4 stereoisomers; (4) Δ^{3,7}-cannabidiol has 2 chiral centers and 4 isomers; (5) Δ²-cannabidiol has 2 chiral centers and 4 stereoisomers; (6) Δ¹-cannabidiol has 2 chiral centers and 4 stereoisomers; and (7) g-cannabidiol has 1 chiral center and 2 stereoisomers. In a preferred embodiment, canabidiol is specifically g-cannabidiol. Unless specifically stated, a reference to “cannabidiol,” “CBD,” or “cannabidiols” or to any of specific cannabidiol compounds (1)-(7) as referred to above includes all possible stereoisomers of all compounds included by the reference. In one embodiment, “Δ²-cannabidiol” can be a mixture of the Δ²-cannabidiol stereoisomers that are partially or entirely produced in a heterologous system.

The term “isoprenoid” or “terpenoid” refers to any compound comprising one or more five-carbon isoprene building blocks, including linear and cyclic terpenoids. As used herein, the term “terpene” is interchangeable with terpenoid and isoprenoid. When terpenes are modified chemically, such as by oxidation or rearrangement of the carbon chain, the resulting compounds are generally referred to as terpenoids, also called isoprenoids.

Terpenoids can be named according to the number of carbon atoms present, using groups of 5 and 10 carbons as a reference. For example a hemiterpenoid (C5) has one isoprene unit (a half-terpenoid); a monoterpenoid (C10) has two isoprene units (one terpenoid); a sesquiterpenoid (C15) has three isoprene units (1.5 terpenoids); and a diterpenoid (C20) has four isoprene units (or two terpenoids). Typically, a monoterpenoid is produced in nature from the C10 terpenoid precursor geranyl pyrophosphate (GPP) Similarly, a “cyclic monoterpene” refers to a cyclic or aromatic terpenoid (i.e., comprising a ring structure). It is made from two isoprene building blocks, typically from GPP. Linear monoterpenes include but are not limited to geraniol, linalool, ocimene, and myrcene. Cyclic monoterpenes (monocyclic, bicyclic and tricyclic) include, but are not limited to, limonene, pinene, carene, terpineol, terpinolene, phellandrene, thujene, tricyclene, borneol, sabinene, and camphene.

A “terpenoid synthase” refers to an enzyme capable of catalyzing the conversion of one terpenoid or terpenoid precursor to another terpenoid or terpenoid precursor. For example, a GPP synthase is an enzyme that catalyzes the formation of GPP, e.g. from the terpenoid precursors IPP and DMAPP. Similarly, an FPP synthase is an enzyme that catalyzes the production of FPP, e.g. from GPP and IPP. Terpene synthases are enzymes that catalyze the conversion of a prenyl diphosphate (such as GPP) into an isoprenoid or an isoprenoid precursor. The term includes both linear and cyclic terpene synthases.

A “cyclic terpenoid synthase” refers to an enzyme capable of catalyzing a reaction that modifies a terpenoid or terpenoid precursor to provide a ring structure. For example, a cyclic monoterpenoid synthase refers to an enzyme capable of using a linear monoterpene as a substrate to produce a cyclic or aromatic (ring-containing) monoterpenoid compound. One example would be sabinene synthase, which is capable of catalyzing the formation of the cyclic monoterpene sabinene from the linear monoterpene precursor GPP. As used herein, the term “terpene synthase” is interchangeable with terpenoid synthase.

A prenyl transferase or isoprenyl transferase enzyme, also called a prenyl or isoprenyl synthase is an enzyme capable of catalyzing the production of a pyrophosphate precursor of a terpenoid or isoprenoid compound. An exemplary prenyl transferase or isoprenyl transferase enzyme is ispA, which is capable of catalyzing the formation of geranyl diphosphate (GPP) or farnesyl diphosphate (FPP) in the presence of a suitable substrate.

An aromatic prenyl transferase is an enzyme capable of catalyzing the transfer of a prenyl group to an aromatic substrate. An exemplary aromatic prenyl transferase is CBGAS. Another exemplary aromatic prenyl transferase is NphB. Yet another exemplary aromatic prenyltransferase is CsPT4.

A “cannabinoid synthase” refers to an enzyme that catalyzes one or more of the following activities: cyclization of CBGA to THCA, CBDA, or CBCA; cyclization of CBGVA to THCVA, CBCVA, CBDVA, prenylation of olivetolate to form CBGA, and combinations thereof. Exemplary cannabinoid synthases include, but are not limited to those found naturally occurring in a plant of the genus Cannabis, such as THCA synthase, CBDA synthase, and CBCA synthase of Cannabis sativa.

Exemplary isoprenoid, terpenoid, cannabinoid, and MEP pathway polypeptides and nucleic acids include those described in the KEGG database. The KEGG database contains the amino acid and nucleic acid sequences of numerous exemplary isoprenoid, terpenoid, cannabinoid, and MEP pathway polypeptides and nucleic acids (see, for example, the world-wide web at “genome.jp/kegg/pathway/map/map00100.html” and the sequences therein, which are each hereby incorporated by reference in their entireties, particularly with respect to the amino acid and nucleic acid sequences of isoprenoid, terpenoid, cannabinoid, and MEP pathway polypeptides and nucleic acids).

As used herein, the term “heterologous” refers to any two components that are not naturally found together. For example, a nucleic acid encoding a gene that is heterologous to an operably linked promoter is a nucleic acid having expression that is not controlled in its natural state (e.g., within a non-genetically modified cell) by the promoter to which it is operably linked in a particular genome. As provided herein, all genes operably linked to non-naturally occurring promoters are considered “heterologous.” Similarly, a gene that is “heterologous” to a host cell is a gene that is not found in a non-genetically modified cell of a particular organism or that is found in a different genomic or non-genomic (e.g., plasmid) location, or operably linked to a different promoter in the non-genetically modified cell. Additionally, a promoter that is “heterologous” to a host cell is a promoter that is not found in a non-genetically modified cell of a particular organism or that is found in a different genomic or non-genomic (e.g., plasmid) location, or operably linked to a different nucleic acid in the non-genetically modified cell.

As used herein, an “expression cassette” refers to the polynucleotide sequences comprising a promoter polynucleotide operably linked to at least one target gene, wherein the promoter is heterologous to at least one operably-linked gene, the promoter is heterologous to a host cell in which it resides, or at least one operably-linked gene is heterologous to the host cell, or a combination thereof. It is understood that in embodiments that describe an expression cassette containing a promoter operably linked to a nucleic acid that encodes two or more proteins, alternative embodiments in which the two or more proteins are in different expression cassettes are also contemplated. Similarly, it is understood that separate expression cassettes can be combined. In typical embodiments, one or more, or all expression cassettes include a promoter operably linked to a codon optimized nucleic acid encoding one or more polypeptides. In an exemplary embodiment, the nucleic acid encoding the heterologous transporter is codon optimized.

“Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate.” Examples of hydrate include, but are not limited to, hemihydrate, monohydrate, dihydrate, trihydrate, hexahydrate, and other water-containing species. It should be understood by one of ordinary skill in the art that the pharmaceutically acceptable salt, and/or prodrug of a compound may also exist in a solvate form. The solvate is typically formed via hydration which is either part of the preparation of a compound or through natural absorption of moisture by an anhydrous compound of the present invention. In general, all physical forms are intended to be within the scope of the present invention.

Thus, when a therapeutically active agent made in a method according to the present invention or included in a composition according to the present invention, such as, but not limited to, a cannabinoid or a terpenoid, possesses a sufficiently acidic, a sufficiently basic, or both a sufficiently acidic and a sufficiently basic functional group, these group or groups can accordingly react with any of a number of inorganic or organic bases, and inorganic and organic acids, to form a pharmaceutically acceptable salt. Exemplary pharmaceutically acceptable salts include those salts prepared by reaction of the pharmacologically active compound with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, β-hydroxybutyrates, glycolates, tartrates, methane-sulfonates, propanesulfonates, naphthalene-1-sulfonates, naphthalene-2-sulfonates, and mandelates. If the pharmacologically active compound has one or more basic functional groups, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the pharmacologically active compound has one or more acidic functional groups, the desired pharmaceutically acceptable salt may be prepared by any suitable method available in the art, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

“Composition” as used herein is intended to encompass a product comprising the specified ingredients in the specified amounts, as well as any product that results from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. One of skill in the art will recognize that other pharmaceutical excipients are useful in the present invention.

In some cases, protecting groups can be included in compounds used in methods according to the present invention or in compositions according to the present invention. The use of such a protecting group is to prevent subsequent hydrolysis or other reactions that can occur in vivo and can degrade the compound. Groups that can be protected include alcohols, amines, carbonyls, carboxylic acids, phosphates, and terminal alkynes. Protecting groups useful for protecting alcohols include, but are not limited to, acetyl, benzoyl, benzyl, β-methoxyethoxyethyl ether, dimethoxytrityl, methoxymethyl ether, methoxytrityl, p-methoxybenzyl ether, methylthiomethyl ether, pivaloyl, tetrahydropyranyl, tetrahydrofuran, trityl, silyl ether, methyl ether, and ethoxyethyl ether. Protecting groups useful for protecting amines include carbobenzyloxy, p-methoxybenzylcarbonyl, t-butyloxycarbonyl, 9-fluorenylmethyloxycarbonyl, acetyl, benzoyl, benzyl, carbamate, p-methoxybenzyl, 3,4-dimethoxybenzyl, p-methoxyphenyl, tosyl, trichloroethyl chloroformate, and sulfonamide Protecting groups useful for protecting carbonyls include acetals, ketals, acylals, and dithianes. Protecting groups useful for protecting carboxylic acids include methyl esters, benzyl esters, t-butyl esters, esters of 2,6-disubstituted phenols, silyl esters, orthoesters, and oxazoline. Protecting groups useful for protecting phosphate groups include 2-cyanoethyl and methyl. Protecting groups useful for protecting terminal alkynes include propargyl alcohols and silyl groups. Other protecting groups are known in the art.

As used herein, the term “prodrug” refers to a precursor compound that, following administration, releases the biologically active compound in vivo via some chemical or physiological process (e.g., a prodrug on reaching physiological pH or through enzyme action is converted to the biologically active compound). A prodrug itself may either lack or possess the desired biological activity. Thus, the term “prodrug” refers to a precursor of a biologically active compound that is pharmaceutically acceptable. n certain cases, a prodrug has improved physical and/or delivery properties over a parent compound from which the prodrug has been derived. The prodrug often offers advantages of solubility, tissue compatibility, or delayed release in a mammalian organism (H. Bundgard, Design of Prodrugs (Elsevier, Amsterdam, 1988), pp. 7-9, 21-24). A discussion of prodrugs is provided in T. Higuchi et al., “Pro-Drugs as Novel Delivery Systems,” ACS Symposium Series, Vol. 14 and in E. B. Roche, ed., Bioreversible Carriers in Drug Design (American Pharmaceutical Association & Pergamon Press, 1987). Exemplary advantages of a prodrug can include, but are not limited to, its physical properties, such as enhanced drug stability for long-term storage.

The term “prodrug” is also meant to include any covalently bonded carriers which release the active compound in vivo when the prodrug is administered to a subject. Prodrugs of a therapeutically active compound, as described herein, can be prepared by modifying one or more functional groups present in the therapeutically active compound, including cannabinoids, terpenoids, and other therapeutically active compounds used in methods according to the present invention or included in compositions according to the present invention, in such a way that the modifications are cleaved, either in routine manipulation or in vivo, to yield the parent therapeutically active compound. Prodrugs include compounds wherein a hydroxy, amino, or mercapto group is covalently bonded to any group that, when the prodrug of the active compound is administered to a subject, cleaves to form a free hydroxy, free amino, or free mercapto group, respectively. Examples of prodrugs include, but are not limited to, formate or benzoate derivatives of an alcohol or acetamide, formamide or benzamide derivatives of a therapeutically active agent possessing an amine functional group available for reaction, and the like.

For example, if a therapeutically active agent or a pharmaceutically acceptable form of a therapeutically active agent contains a carboxylic acid functional group, a prodrug can comprise an ester formed by the replacement of the hydrogen atom of the carboxylic acid group with a group such as C₁₋₈ alkyl, C₂₋₁₂ alkanoyloxymethyl, 1-(alkanoyloxy)ethyl having from 4 to 9 carbon atoms, 1-methyl-1-(alkanoyloxy)ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N(C₁-C₂)alkylamino(C₂-C₃)alkyl (such as (3-dimethylaminoethyl), carbamoyl-(C₁-C₂)alkyl, N,N-di (C₁-C₂)alkylcarbamoyl-(C₁-C₂)alkyl and piperidino-, pyrrolidino-, or morpholino(C₂-C₃)alkyl.

Similarly, if a disclosed compound or a pharmaceutically acceptable form of the compound contains an alcohol functional group, a prodrug can be formed by the replacement of the hydrogen atom of the alcohol group with a group such as (C₁-C₆)alkanoyloxymethyl, 1-((C₁-C₆))alkanoyloxy)ethyl, 1-methyl-1-((C₁-C₆)alkanoyloxy)ethyl (C₁-C₆)alkoxycarbonyloxymethyl, N(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, P(O)(O(C₁-C₆)alkyl)₂ or glycosyl (the radical resulting from the removal of a hydroxyl group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, C(OH)C(O)OY¹ wherein Y¹ is H, (C₁-C₆)alkyl or benzyl, C(OY²)Y³ wherein Y² is (C₁-C₄) alkyl and Y³ is (C₁-C₆)alkyl, carboxy(C₁-C₆)alkyl, amino(C₁-C₄)alkyl or mono-N or di-N,N(C₁-C₆)alkylaminoalkyl, C(Y⁴)Y⁵ wherein Y⁴ is H or methyl and Y⁵ is mono-N or di-N,N(C₁-C₆)alkylamino, morpholino, piperidin-1-yl or pyrrolidin-1-yl.

The use of prodrug systems is described in T. Järvinen et al., “Design and Pharmaceutical Applications of Prodrugs” in Drug Discovery Handbook (S. C. Gad, ed., Wiley-Interscience, Hoboken, N.J., 2005), ch. 17, pp. 733-796. Other alternatives for prodrug construction and use are known in the art. When a method or pharmaceutical composition according to the present invention, uses or includes a prodrug of a cannabinoid, terpenoid, or other therapeutically active agent, prodrugs and active metabolites of a compound may be identified using routine techniques known in the art. See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

As used herein, where a polypeptide such as an OMP super family porin, e.g., an OrpD family porin such as pp3656, an MFS aromatic antiporter, an aromatic prenyltransferase, a cannabinoid synthase, and/or a non-mevalonate pathway component are disclosed or claimed, it will be appreciated that orthologues of the recited polypeptide are alternatively contemplated.

### Cannabinoids

Cannabinoids are a group of chemicals known to activate cannabinoid receptors in cells throughout the human body, including the skin. Phytocannabinoids are the cannabinoids derived from cannabis plants. They can be isolated from plants or produced synthetically. Endocannabinoids are endogenous cannabinoids found in the human body. Canonical phytocannabinoids are ABC tricyclic terpenoid compounds bearing a benzopyran moiety.

Cannabinoids exert their effects by interacting with cannabinoid receptors present on the surface of cells. To date, two types of cannabinoid receptor have been identified, the CB1 receptor and the CB2 receptor. These two receptors share about 48% amino acid sequence identity, and are distributed in different tissues and also have different signaling mechanisms. They also differ in their sensitivity to agonists and antagonists.

Accordingly, in vitro and in vivo methods are described herein for screening for, identifying, making, and using genes, promoters, helper pathway components, and expression cassettes for in vivo production of cannabinoids.

Typically, the methods and compositions described herein can be used for production, or increased production of one or more cannabinoids in a host cell, or production of one or more cannabinoid precursors in a host cell. In some cases, the cannabinoids or precursors thereof, can be purified, derivatized (e.g., to form a prodrug, solvate, or salt, or to form a target cannabinoid from the precursor), and/or formulated in a pharmaceutical composition.

The cannabinoids that can be produced according to the methods and/or using the compositions of the present invention include but are not limited to phytocannabinoids. In some cases the cannabinoids include but are not limited to, cannabinol, cannabidiols, g-tetrahydrocannabinol (Δ⁹-THC), the synthetic cannabinoid HU-210 (6aR,10aR)-9-(hydroxymethyl)-6,6-dimethyl-3-(2-methyloctan-2-yl)-6H,6aH,7H,10H,10aH-benzo[c] isochromen-1-ol), cannabidivarin (CBDV), cannabichromene (CBC), cannabichromevarin (CBCV), cannabigerol (CBG), cannabigerovarin (CBGV), cannabielsoin (CBE), cannabicyclol (CBL), cannabivarin (CBV), and cannabitriol (CBT). Still other cannabinoids include, including tetrahydrocannibivarin (THCV) and cannabigerol monomethyl ether (CBGM). Additional cannabinoids include cannabichromenic acid (CBCA), g-tetrahydrocannabinolic acid (THCA); and cannabidiolic acid (CBDA); these additional cannabinoids are characterized by the presence of a carboxylic acid group in their structure.

Still other cannabinoids include nabilone, rimonabant, JWH-018 (naphthalen-1-yl-(1-pentylindol-3-yl)methanone), JWH-073 naphthalen-1-yl-(1-butylindol-3-yl)methanone, CP-55940 (2-[(1R,2R,5R)-5-hydroxy-2-(3-hydroxypropyl) cyclohexyl]-5-(2-methyloctan-2-yl)phenol), dimethylheptylpyran, HU-331 (3-hydroxy-2-[(1R)-6-isopropenyl-3-methyl-cyclohex-2-en-1-yl]-5-pentyl-1,4-benzoquinone), SR144528 (5-(4-chloro-3-methylphenyl)-1-[(4-methylphenyl)methyl]-N-[(1S,2S,4R)-1,3,3-trimethylbicyclo[2.2.1]heptan-2-yl]-1H-pyrazole-3-carboxamide), WIN 55,212-2 ((11R)-2-methyl-11-[(morpholin-4-yl)methyl]-3-(naphthalene-1-carbonyl)-9-oxa-1-azatricyclo[6.3.1.0^{4,12}]dodeca-2,4(12),5,7-tetraene), JWH-133 ((6aR,10aR)-3-(1,1-dimethylbutyl)-6a,7,10,10a-tetrahydro-6,6,9-trimethyl-6H-dibenzo[b,d]pyran), levonatradol, and AM-2201 (1-[(5-fluoropentyl)-1H-indol-3-yl]-(naphthalen-1-yl)methanone). Other cannabinoids include g-tetrahydrocannabinol (g-THC), 11-hydroxy-Δ⁹-tetrahydrocannabinol, Δ¹¹-tetrahydrocannabinol, and 11-hydroxy-tetracannabinol.

In another alternative, analogs or derivatives of these cannabinoids can be obtained by production of cannabinoid precursors and further derivatization, e.g., by synthetic means. Synthetic cannabinoids include, but are not limited to, those described in U.S. Pat. No. 9,394,267 to Attala et al.; U.S. Pat. No. 9,376,367 to Herkenroth et al.; U.S. Pat. No. 9,284,303 to Gijsen et al.; U.S. Pat. No. 9,173,867 to Travis; U.S. Pat. No. 9,133,128 to Fulp et al.; U.S. Pat. No. 8,778,950 to Jones et al.; U.S. Pat. No. 7,700,634 to Adam-Worrall et al.; U.S. Pat. No. 7,504,522 to Davidson et al.; U.S. Pat. No. 7,294,645 to Barth et al.; U.S. Pat. No. 7,109,216 to Kruse et al.; U.S. Pat. No. 6,825,209 to Thomas et al.; and U.S. Pat. No. 6,284,788 to Mittendorf et al.

In another alternative, the cannabinoid can be an endocannabinoid or a derivative or analog thereof. Endocannabinoids include but are not limited to anandamide, 2-arachidonoylglycerol, 2-arachidonyl glyceryl ether, N-arachidonoyl dopamine, and virodhamine A number of analogs of endocannabinoids are known, including 7,10,13,16-docosatetraenoylethanolamide, oleamide, stearoylethanolamide, and homo-γ-linolenoylethanolamine, are also known.

Cannabinoids produced in methods and compositions according to the present invention can be either selective for the CB2 cannabinoid receptor or non-selective for the two cannabinoid receptors, binding to either the CB1 cannabinoid receptor or the CB2 cannabinoid receptor. In some cases, cannabinoids produced in methods and compositions according to the present invention are selective for the CB2 cannabinoid receptor. In some cases, the cannabinoids, or one of the cannabinoids in a mixture of cannabinoids is an antagonist (e.g., selective or non-selective antagonist) of CB2. In some cases, cannabinoids produced in methods and compositions according to the present invention are selective for the CB2 cannabinoid receptor. In some cases, the cannabinoids, or one of the cannabinoids in a mixture of cannabinoids is an antagonist (e.g., selective or non-selective antagonist) of CB1.

### Expression Cassettes

Described herein are expression cassettes suitable for expressing one or more target genes in a host cell. The expression cassettes described herein can be a component of a plasmid or integrated into a host cell genome. A single plasmid can contain one or more expression cassettes described herein. As used herein, where two or more expression cassettes are described, it is understood that alternatively at least two of the two or more expression cassettes can be combined to reduce the number of expression cassettes. Similarly, where multiple target genes are described as operably linked to a single promoter and thus described as components of a single expression cassette, it is understood that the single expression cassette can be sub-divided into two or more expression cassettes containing overlapping or non-overlapping subsets of the single described expression cassette.

An expression cassette described herein can contain a suitable promoter as known in the art. In some cases, the promoter is a constitutive promoter. In other cases, the promoter is an inducible promoter. In preferred embodiments in, or for use in, a prokaryotic host, the promoter is a T5 promoter, a T7 promoter, a Trc promoter, a Lac promoter, a Tac promoter, a Trp promoter, a tip promoter, a λP_{L} promoter, a λP_{R} promoter, a λP_{R}P_{L} promoter, an arabinose promoter (araBAD), and the like. In some embodiments, the promoter is selected from the group consisting of the promoters described in Lee et al., Applied and Environmental Microbiology, September 2007, p. 5711-15, which is hereby incorporated by reference in the entirety, particularly with respect to promoters, expression cassettes, including plasmids, for the expression of nucleic acids of interest, target genes, host cells, and combinations thereof described therein. In some embodiments, the promoter is selected from the group consisting of the E. coli promoters described in Zaslaver et al., Nat Methods. 2006 August; 3(8):623-8, which is hereby incorporated by reference in the entirety, particularly with respect to promoters, expression cassettes, including plasmids, for the expression of nucleic acids of interest, target genes, host cells, and combinations thereof described therein. Promoters useful to drive expression of one or more target genes in various host cells are numerous and familiar to those skilled in the art (see, for example, WO 2004/033646; U.S. Pat. Nos. 8,507,235; 8,715,962; and WO 2011/017798, and references cited therein, which are each hereby incorporated by reference in their entireties, particularly with respect to promoters, expression cassettes, including plasmids, for the expression of nucleic acids of interest, target genes, host cells, and combinations thereof described therein.

Methods and compositions described herein can be used for expression of a functional heterologous transporter such as an MFS aromatic acid antiporter (e.g., pcaK) or an OMP superfamily porin such as a porin of the OprD family (e.g., pp3656). Methods and compositions described herein can additionally be used for expression of a functional aromatic prenyltransferase. In some cases, methods and compositions described herein can additionally be used to increase production of a prenyl donor, e.g., via the non-mevalonate pathway such as by expression of a bifunctional ispDF enzyme and/or a bifunctional ispDE enzyme. Methods and compositions described herein can additionally be used for expression of a functional cannabinoid synthase such as THCAS and/or CBDAS.

Typically, the functional THCAS and/or CBDAS is provided by co-expression of one or more helper pathway components and/or one or more components of one or more helper pathways.

The heterologous transporter can be modified for expression in a host. For example, one or more transmembrane or signal peptide domains can be truncated or substituted for a transmembrane or signal peptide domain compatible with expression in the host cell. Additionally, or alternatively, one or more glycosylation sites can be deleted (e.g., by mutation of the primary amino acid sequence). Similarly, one or more or all cysteines found in an intramolecular disulfide bond in the native protein in its native host can be mutated, e.g., to serine. Similarly, one or more or all cysteines found in an intermolecular disulfide bond in the native protein in its native host can be mutated, e.g., to serine.

Methods and compositions described herein can be used for expression of a GPP synthase in a suitable (e.g., prokaryotic) host cell in combination with expression of the heterologous transporter and optionally the aromatic prenyltransferase. For example, the host cell can comprise an expression cassette having a promoter operably linked to a heterologous nucleic acid encoding a GPP synthase.

Methods and compositions described herein can be used for expression of one or more genes of the MEP pathway in a suitable (e.g., prokaryotic) host cell in combination with expression of the heterologous transporter and optionally the aromatic prenyltransferase. In some embodiments, MEP pathway flux is increased by overexpression of one or more endogenous components of the host cell by amplification of gene copy number and/or operably linking an endogenous gene (or copy thereof) to a strong constitutive or inducible heterologous promoter. Accordingly, in one embodiment, an expression cassette comprising a promoter operably linked to a nucleic acid encoding one or more genes of the MEP pathway is provided. In E. coli, endogenous MEP pathway genes are dxs, ispC, ispD, ispE, ispF, ispG, ispH, and idi.

In some cases, the promoter of the expression cassette is operably linked to a nucleic acid encoding two or more genes of the MEP pathway. In some cases, the promoter of the expression cassette is operably linked to a nucleic acid encoding three or more genes of the MEP pathway. In some cases, the promoter of the expression cassette is operably linked to a nucleic acid encoding four, five, six, or all endogenous genes of the MEP pathway, or orthologues of one, two, three, four, five, six, or all thereof. In some cases, the genes of the MEP pathway provided in the expression cassette are prokaryotic genes. In some cases, the genes of the MEP pathway provided in the expression cassette are E. coli genes. In other cases, one or more of the genes of the MEP pathway provided in the expression cassette are genes that are heterologous to wild-type E. coli. In some cases, one or more genes of the MEP pathway are provided in a first expression cassette and one or more genes of the MEP pathway are provided in a second expression cassette. In a preferred embodiment, an expression cassette comprising a promoter operably linked to dxs and idi is provided.

In some cases, an expression cassette is provided that comprises a promoter operably linked to a nucleic acid encoding one or more genes of the MEP pathway and further encoding a GPP synthase, a cannabinoid synthase, or an isoprene synthase, or a functional fragment thereof. In some cases, an expression cassette is provided that comprises a promoter operably linked to a nucleic acid encoding one or more genes of the MEP pathway and further encoding THCA synthase or a functional fragment thereof. In some cases, an expression cassette is provided that comprises a promoter operably linked to a nucleic acid encoding one or more genes of the MEP pathway and further encoding CBGA synthase or a functional fragment thereof. In some cases, an expression cassette is provided that comprises a promoter operably linked to a nucleic acid encoding one or more genes of the MEP pathway and further encoding CBDA synthase or a functional fragment thereof. In some cases, an expression cassette is provided that comprises a promoter operably linked to a nucleic acid encoding one or more genes of the MEP pathway and further encoding NphB or a functional fragment thereof.

In some embodiments, an expression cassette containing a promoter operably linked to a nucleic acid encoding a bifunctional ispDF enzyme is provided. The ispDF gene can be used in addition to, or as an alternative to, overexpression of native ispD and/or ispF in the host cell. In some cases, the nucleic acid encodes an ispDF protein having the following amino acid sequence (SEQ ID NO. 5): MIALQRSLSMHVTAIIAAAGEGRRLGAPLPKQLLDIGGRSILERSVMAFARHERIDDVIVVLPPAL AAAPPDWIAASGRVPAVHVVSGGERRQDSVANAFDRVPAQSDVVLVHDAARPFVTAELISRAI DGAMQHGAAIVAVPVRDTVKRVDPDGEHPVITGTIPRDTIYLAQTPQAFRRDVLGAAVALGRSG VSATDEAMLAEQAGHRVHVVEGDPANVKITTSADLDQARQRLRSAVAARIGTGYDLHRLIEGR PLIIGGVAVPCDKGALGHSDADVACHAVIDALLGAAGAGNVGQHYPDTDPRWKGASSIGLLRD ALRLVQERGFTVENVDVCVVLERPKIAPFIPEIRARIAGALGIDPERVSVKGKTNEGVDAVGRGE AIAAHAVALLSES.

In other embodiments, the ispDF nucleic acid encodes an ispDF protein identical to, or having at least 32%, 40%, 45%, 50%, 52%, 55%, 60%, 65%, 70%, 80%, 85%, 90%, 95%, 99% identity with respect to, SEQ ID NO.5.

In some cases, the bifunctional ispDF has a primary amino acid sequence that is no more than 75% identical to at least 300 contiguous amino acids of H. pylori HP1020, H. pylori HP1020, H. pylori J99 jhp0404, H. pylori HPAG1 HPAG1_0427, H. hepaticus HH1582, H. acinonychis st. Sheeba Hac_1124, W. succinogenes DSM 1740 WS1940, S. denitrificans DSM 1251 Suden_1487, C. jejuni subsp. jejuni NCTC 11168 Cj1607, C. jejuni RM1221 CJE1779, C. jejuni subsp. jejuni 81-176 CH81176_1594, and C. fetus subsp. fetus 82-40 CFF8240_0409. In some cases, the bifunctional ispDF is not H. pylori HP1020, H. pylori HP1020, H. pylori J99 jhp0404, H. pylori HPAG1 HPAG1_0427, H hepaticus HH1582, H. acinonychis st. Sheeba Hac_1124, W. succinogenes DSM 1740 WS1940, S. denitrificans DSM 1251 Suden_1487, C. jejuni subsp. jejuni NCTC 11168 Cj1607, C. jejuni RM1221 CJE1779, C. jejuni subsp. jejuni 81-176 CH81176_1594, or C. fetus subsp. fetus 82-40 CFF8240_0409.

Exemplary ispDF bifunctional enzymes are described herein. Further examples of bifunctional ispDF enzymes include but are not limited to those illustrated in the table below:

| Fusion | Sequence for IspD domain | Sequence for IspF domain |
|---|---|---|
| IspDF₁ | MIALQRSLSMHVTAIIAAAGEGRRLGAPLPK | RIGTGYDLHRLIEGRPLIIGGVAVP |
| | QLLDIGGRSILERSVMAFARHERIDDVIVVLP | CDKGALGHSDADVACHAVIDALL |
| | PALAAAPPDWIAASGRVPAVHVVSGGERRQ | GAAGAGNVGQHYPDTDPRWKGA |
| | DSVANAFDRVPAQSDVVLVHDAARPFVTAE | SSIGLLRDALRLVQERGFTVENVD |
| | LISRAIDGAMQHGAAIVAVPVRDTVKRVDP | VCVVLERPKIAPFIPEIRARIAGAL |
| | DGEHPVITGTIPRDTIYLAQTPQAFRRDVLGA | GIDPERVSVKGKTNEGVDAVGRG |
| | AVALGRSGVSATDEAMLAEQAGHRVHVVE | EAIAAHAVALLSES |
| | GDPANVKITTSADLDQA | |
| IspDF₂ | MQVTAIIAAGGRGRRFGGGVPKQLVGVGGR | FRIGAGYDLHRLVEGRPLVLGGV |
| | PILERTVAAFLGHPAIHEVVVALPAELMADP | TIPFERGLLGHSDADAICHAVTDA |
| | PAYLRAAPKPIRLVAGGVQRQDSVRQAFQA | VLGAAAAGDIGRHFPDSDPKWRD |
| | ANEQSDVIVIHDAARPFASADLISRTIAAAAE | WSSIDLLRRASAIVKGRGYAIANV |
| | GGAALAAVPARDTVKRGAFAAGRTGPAGR | DAVVIAERPKLAPFLDEMRANVA |
| | QAVEGAPLLVVAETLPRDSIYLAQTPQAFRR | GAIGIAVDAVGIKGKTNEGLGELG |
| | DVLRDALALGEAGSEATDEATLAERAGHIV | RGEAIAVHAVALLHL |
| | RLVEGEPANIKITTPDDLLVA | |
| IspDF₃ | MVHVSAIIAAGGRGERFGGPQPKQLLLLGG | RIGNGYDLHRLVTGRPLVLGGVTI |
| | VPILKRTVDAFLRGYPFIEVIVALPAEFVANP | PFEKGLQGHSDADAVCHAITDAIL |
| | PDYLDDVIVVEGGARRQDSVANAFRAVAPS | GAASAGDIGRHFPDTDPAWKDAK |
| | AQVVVIHDAARPLVTPSLIERTVDAAVKHG | SIVLLQQAAQIVSRAGYAIANLDV |
| | AAIAALRATDTVKRGDASRVIRGTLPRDEIFL | VVIAQQPKLVPHIDAIRHSVAHAL |
| | AQTPQAFRAGVLRDALALAASAADATDEA | GIDVQQVSVKGKTNEGVDSMGA |
| | MLAEQAGHHVRLVDGDPRNLKITTPEDLEM | GESIAVHAVALLQHS |
| | A | |

| Fusion | Amino Acid Sequence |
|---|---|
| ispD_{CJ}F | MATTHLDVCAVVPAAGFGRRMQTECPKQYLSIGNQTILEHSVHALLAHPRVKRVV |
| | IAISPGDSRFAQLPLANHPQITVVDGGDERADSVLAGLKAAGDAQWVLVHDAARP |
| | CLHQDDLARLLALSETSRTGGILAAPVRDTMKRAEPGKNAIAHTVDRNGLWHALT |
| | PQFFPRELLHDCLTRALNEGATITDEASALEYCGFHPQLVEGRADNIKVTRPEDLAL |
| | AEFYLTLPTPSFEIRIGHGFDVHAFGGEGPIIIGGVRIPYEKGLLAHSDGDVALHALT |
| | DALLGAAALGDIGKLFPDTDPAFKGADSRELLREAWRRIQAKGYTLGNVDVTIIAQ |
| | APKMLPHIPQMRVFIAEDLGCHMDDVNVKATTTEKLGFTGRGEGIACEAVALLIKA |
| | TK |
| ispD_{FL}F | MATTHLDVCAVVPAAGFGRRMQTECPKQYLSIGNQTILEHSVHALLAHPRVKRVV |
| | IAISPGDSRFAQLPLANHPQITVVDGGDERADSVLAGLKAAGDAQWVLVHDAARP |
| | CLHQDDLARLLALSETSRTGGILAAPVRDTMKRAEPGKNAIAHTVDRNGLWHALT |
| | PQFFPRELLHDCLTRALNEGATITDEASALEYCGFHPQLVEGRADNIKVTRPEDLAL |
| | AEFYLSLGGGGSAAAIGHGFDVHAFGGEGPIIIGGVRIPYEKGLLAHSDGDVALHAL |
| | TDALLGAAALGDIGKLFPDTDPAFKGADSRELLREAWRRIQAKGYTLGNVDVTIIA |
| | QAPKMLPHIPQMRVFIAEDLGCHMDDVNVKATTTEKLGFTGRGEGIACEAVALLIK |
| | ATK |
| ispD_{RL}F | MATTHLDVCAVVPAAGFGRRMQTECPKQYLSIGNQTILEHSVHALLAHPRVKRVV |
| | IAISPGDSRFAQLPLANHPQITVVDGGDERADSVLAGLKAAGDAQWVLVHDAARP |
| | CLHQDDLARLLALSETSRTGGILAAPVRDTMKRAEPGKNAIAHTVDRNGLWHALT |
| | PQFFPRELLHDCLTRALNEGATITDEASALEYCGFHPQLVEGRADNIKVTRPEDLAL |
| | AEFYLAEAAAKEAAAKEAAAKEAAAKEAAAKAAAIGHGFDVHAFGGEGPIIIGGV |
| | RIPYEKGLLAHSDGDVALHALTDALLGAAALGDIGKLFPDTDPAFKGADSRELLRE |
| | AWRRIQAKGYTLGNVDVTIIAQAPKMLPHIPQMRVFIAEDLGCHMDDVNVKATTT |
| | EKLGFTGRGEGIACEAVALLIKATK |
| ispD_{XL}F | MATTHLDVCAVVPAAGFGRRMQTECPKQYLSIGNQTILEHSVHALLAHPRVKRVV |
| | IAISPGDSRFAQLPLANHPQITVVDGGDERADSVLAGLKAAGDAQWVLVHDAARP |
| | CLHQDDLARLLALSETSRTGGILAAPVRDTMKRAEPGKNAIAHTVDRNGLWHALT |
| | PQFFPRELLHDCLTRALNEGATITDEASALEYCGFHPQLVEGRADNIKVTRPEDLAL |
| | AEFYLRQRLRSAVAAIGHGFDVHAFGGEGPIIIGGVRIPYEKGLLAHSDGDVALHAL |
| | TDALLGAAALGDIGKLFPDTDPAFKGADSRELLREAWRRIQAKGYTLGNVDVTIIA |
| | QAPKMLPHIPQMRVFIAEDLGCHMDDVNVKATTTEKLGFTGRGEGIACEAVALLIK |
| | ATK |
| ispD_{CJ}F₁ | MIALQRSLSMHVTAIIAAAGEGRRLGAPLPKQLLDIGGRSILERSVMAFARHERIDD |
| | VIVVLPPALAAAPPDWIAASGRVPAVHVVSGGERRQDSVANAFDRVPAQSDVVLV |
| | HDAARPFVTAELISRAIDGAMQHGAAIVAVPVRDTVKRVDPDGEHPVITGTIPRDTI |
| | YLAQTPQAFRRDVLGAAVALGRSGVSATDEAMLAEQAGHRVHVVEGDPANVKIT |
| | TSADLDQADLPTPSFERIGTGYDLHRLIEGRPLIIGGVAVPCDKGALGHSDADVACH |
| | AVIDALLGAAGAGNVGQHYPDTDPRWKGASSIGLLRDALRLVQERGFTVENVDVC |
| | VVLERPKIAPFIPEIRARIAGALGIDPERVSVKGKTNEGVDAVGRGEAIAAHAVALLS |
| | ES |
| ispD_{FL}F₁ | MIALQRSLSMHVTAIIAAAGEGRRLGAPLPKQLLDIGGRSILERSVMAFARHERIDD |
| | VIVVLPPALAAAPPDWIAASGRVPAVHVVSGGERRQDSVANAFDRVPAQSDVVLV |
| | HDAARPFVTAELISRAIDGAMQHGAAIVAVPVRDTVKRVDPDGEHPVITGTIPRDTI |
| | YLAQTPQAFRRDVLGAAVALGRSGVSATDEAMLAEQAGHRVHVVEGDPANVKIT |
| | TSADLDQASLGGGGSAAARIGTGYDLHRLIEGRPLIIGGVAVPCDKGALGHSDADV |
| | ACHAVIDALLGAAGAGNVGQHYPDTDPRWKGASSIGLLRDALRLVQERGFTVENV |
| | DVCVVLERPKIAPFIPEIRARIAGALGIDPERVSVKGKTNEGVDAVGRGEAIAAHAV |
| | ALLSES |
| ispD_{RL}F₁ | MIALQRSLSMHVTAIIAAAGEGRRLGAPLPKQLLDIGGRSILERSVMAFARHERIDD |
| | VIVVLPPALAAAPPDWIAASGRVPAVHVVSGGERRQDSVANAFDRVPAQSDVVLV |
| | HDAARPFVTAELISRAIDGAMQHGAAIVAVPVRDTVKRVDPDGEHPVITGTIPRDTI |
| | YLAQTPQAFRRDVLGAAVALGRSGVSATDEAMLAEQAGHRVHVVEGDPANVKIT |
| | TSADLDQARQRLRSAVLAEAAAKEAAAKEAAAKEAAAKEAAAKAAARIGTGYDL |
| | HRLIEGRPLIIGGVAVPCDKGALGHSDADVACHAVIDALLGAAGAGNVGQHYPDT |
| | DPRWKGASSIGLLRDALRLVQERGFTVENVDVCVVLERPKIAPFIPEIRARIAGALGI |
| | DPERVSVKGKTNEGVDAVGRGEAIAAHAVALLSES |

Exemplary ispDF enzymes further include ispDF enzymes having at least 80% identity (or 85%, or 90%, or 95%, or 99%, or 100% identity) to an ispDF enzyme sequence provided herein (e.g., IspDF₁, IspDF₂, or IspDF₃). Further exemplary ispDF enzymes include ispDF enzymes having an ispF domain at least 80% identical (or 85%, or 90%, or 95%, or 99%, or 100% identical) to the ispF domain sequences provided in the foregoing table. Further exemplary ispDF enzymes include ispDF enzymes having an ispD domain at least 80% identical (or 85%, or 90%, or 95%, or 99%, or 100% identical) to the ispD domain sequences provided in the foregoing table.

The bifunctional ispDF can be encoded by a nucleic acid within a plasmid. Alternatively, the bifunctional ispDF can be encoded by a nucleic acid that is integrated into the genome of a heterologous host cell. In some cases, a heterologous promoter is operably linked to the nucleic acid encoding the bifunctional ispDF. Additionally or alternatively, a host cell can be heterologous to the nucleic acid encoding the bifunctional ispDF. Bifunctional ispDF enzymes and methods of their use in e.g., cannabinoid production in host cells (e.g., prokaryotic host cells) are described, e.g., in PCT/CA2018/051074, the contents of which are incorporated in the entirety for all purposes.

The nucleic acid encoding the bifunctional ispDF can be in an MEP pathway expression cassette such as any one of the foregoing expression cassettes that contain a nucleic acid encoding an MEP pathway gene. In some cases, the nucleic acid encoding the bifunctional ispDF can be in an expression cassette that contains a nucleic acid encoding a cannabinoid synthase. In some cases, the nucleic acid encoding the bifunctional ispDF can be in an expression cassette that contains a nucleic acid encoding GPP synthase. In some cases, the nucleic acid encoding the bifunctional ispDF can be in an expression cassette that contains a nucleic acid encoding an isoprene synthase.

In some embodiments, an expression cassette containing a promoter operably linked to a nucleic acid encoding a bifunctional ispDE enzyme is provided. The ispDE gene can be used in addition to, or as an alternative to, overexpression of native ispD and/or ispF and/or a heterologous ispDF in the host cell. In some cases, the nucleic acid encodes an ispDE protein having a native ispD amino acid sequence, or functional fragment thereof fused via a linker to a native ispE amino acid sequence, or functional fragment thereof.

Exemplary ispDE bifunctional enzymes are described herein. Further examples of bifunctional ispDE enzymes include but are not limited to those illustrated in the table below (linker sequence in bold and underlined):

| Fusion | Sequence of IspD domain | Sequence of IspE domain |
|---|---|---|
| Range | 1 to 236 | 246 to 529 |
| of | | |
| amino | | |
| acid | | |
| IspD_{FL}E | MATTHLDVCAVVPAAGFGRRMQTECPKQY | MRTQWPSPAKLNLFLYITGQRAD |
| | LSIGNQTILEHSVHALLAHPRVKRVVIAISPG | GYHTLQTLFQFLDYGDTISIELRD |
| | DSRFAQLPLANHPQITVVDGGDERADSVLA | DGDIRLLTPVEGVEHEDNLIVRAA |
| | GLKAAGDAQWVLVHDAARPCLHQDDLARL | RLLMKTAADSGRLPTGSGANISID |
| | LALSETSRTGGILAAPVRDTMKRAEPGKNAI | KRLPMGGGLGGGSSNAATVLVAL |
| | AHTVDRNGLWHALTPQFFPRELLHDCLTRA | NHLWQCGLSMDELAEMGLTLGA |
| | LNEGATITDEASALEYCGFHPQLVEGRADNI | DVPVFVRGHAAFAEGVGEILTPV |
| | KVTRPEDLALAEFYLTRTIHQENTSLGGGG | DPPEKWYLVAHPGVSIPTPVIFKD |
| | SAAA | PELPRNTPKRSIETLLKCEFSNDCE |
| | | VIARKRFREVDAVLSWLLEYAPSR |
| | | LTGTGACVFAEFDTESEARQVLEQ |
| | | APEWLNGFVAKGANLSPLHRAML |

Exemplary ispDE enzymes further include ispDE enzymes having at least 80% identity (or 85%, or 90%, or 95%, or 99%, or 100% identity) to an ispDE enzyme sequence provided herein (e.g., SEQ ID NO:10). Further exemplary ispDE enzymes include ispDE enzymes having an ispE domain at least 80% identical (or 85%, or 90%, or 95%, or 99%, or 100% identical) to the ispE domain sequences provided in the foregoing table. Further exemplary ispDE enzymes include ispDE enzymes having an ispD domain at least 80% identical (or 85%, or 90%, or 95%, or 99%, or 100% identical) to the ispD domain sequences provided in the foregoing table (e.g., excluding the linker sequence). Further exemplary ispDE enzymes include ispDE enzymes having an ispD domain at least 80% identical (or 85%, or 90%, or 95%, or 99%, or 100% identical) to the ispD domain sequences provided in the foregoing table including the linker sequence.

The bifunctional ispDE can be encoded by a nucleic acid within a plasmid. Alternatively, the bifunctional ispDE can be encoded by a nucleic acid that is integrated into the genome of a heterologous host cell. In some cases, a heterologous promoter is operably linked to the nucleic acid encoding the bifunctional ispDE. Additionally or alternatively, a host cell can be heterologous to the nucleic acid encoding the bifunctional ispDE.

In some embodiments, an ispEF bifunctional enzyme, or a nucleic acid encoding such an ispEF bifunctional enzyme is provided. Exemplary ispEF bifunctional enzymes include but are not limited those provided in the table below, as well as ispEF bifunctional enzymes having 80% % identity (or 85%, or 90%, or 95%, or 99%, or 100% identity) to an ispEF enzyme sequence described in the table below.

| Fusion | Amino Acid Sequence |
|---|---|
| ispE_{FL}F | MRTQWPSPAKLNLFLYITGQRADGYHTLQTLFQFLDYGDTI |
| | SIELRDDGDIRLLTPVEGVEHEDNLIVRAARLLMKTAADSG |
| | RLPTGSGANISIDKRLPMGGGLGGGSSNAATVLVALNHLWQ |
| | CGLSMDELAEMGLTLGADVPVFVRGHAAFAEGVGEILTPVD |
| | PPEKWYLVAHPGVSIPTPVIFKDPELPRNTPKRSIETLLKC |
| | EFSNDCEVIARKRFREVDAVLSWLLEYAPSRLTGTGACVFA |
| | EFDTESEARQVLEQAPEWLNGFVAKGANLSPLHRAMLSLGG |
| | GGSAAAMRIGHGFDVHAFGGEGPIIIGGVRIPYEKGLLAHS |
| | DGDVALHALTDALLGAAALGDIGKLFPDTDPAFKGADSREL |
| | LREAWRRIQAKGYTLGNVDVTIIAQAPKMLPHIPQMRVFIA |
| | EDLGCHMDDVNVKATTTEKLGFTGRGEGIACEAVALLIKAT |
| | K |

Further exemplary ispEF enzymes include ispEF enzymes having an ispF domain at least 80% identical (or 85%, or 90%, or 95%, or 99%, or 100% identical) to the ispF domain sequence provided in the foregoing table. Further exemplary ispEF enzymes include ispEF enzymes having an ispE domain at least 80% identical (or 85%, or 90%, or 95%, or 99%, or 100% identical) to the ispE domain sequence provided in the foregoing table.

The bifunctional ispEF can be encoded by a nucleic acid within a plasmid. Alternatively, the bifunctional ispEF can be encoded by a nucleic acid that is integrated into the genome of a heterologous host cell. In some cases, a heterologous promoter is operably linked to the nucleic acid encoding the bifunctional ispEF. Additionally or alternatively, a host cell can be heterologous to the nucleic acid encoding the bifunctional ispEF.

In some cases, the nucleic acid encodes an ispDE protein having an ispD amino acid sequence, that is at least 32%, 40%, 45%, 50%, 52%, 55%, 60%, 65%, 70%, 80%, 85%, 90%, 95%, or 99% identical, or is identical, to a functional fragment of an E. coli native ispD amino acid sequence. In some cases, the nucleic acid encodes or further encodes an ispDE protein having an ispE amino acid sequence, that is at least 32%, 40%, 45%, 50%, 52%, 55%, 60%, 65%, 70%, 80%, 85%, 90%, 95%, or 99% identical, or is identical, to a functional fragment of an E. coli native ispE amino acid sequence.

In some cases, the nucleic acid encoding the ispDE protein encodes a flexible peptide linker between the ispE and ispD domains. In some cases, the flexible linker is from 6 to 15 amino acids in length. In some cases, the flexible linker is from 7 to 12 amino acids in length. In some cases, the flexible linker comprises at least 65% or at least 70% random coil formation as predicted by the GOR algorithm, version IV.

The bifunctional ispDE can be encoded by a nucleic acid within a plasmid. Alternatively, the bifunctional ispDE can be encoded by a nucleic acid that is integrated into the genome of a heterologous host cell. In some cases, a heterologous promoter is operably linked to the nucleic acid encoding the bifunctional ispDE. Additionally or alternatively, a host cell can be heterologous to the nucleic acid encoding the bifunctional ispDE.

ispDE bifunctional enzymes described herein can be useful for generating isoprene. ispDE bifunctional enzymes described herein can be useful for generating one or more terpenoids, such as hemiterpenoids, monoterpenoids, sequiterpenoids, diterpenoids, indole diterpenes, triterpenoids, cyclic terpenoids, and linear terpenoids. Exemplary terpenoid products include but are not limited to lycopene, geraniol, linalool, ocimene, and myrcene, taxol, limonene, pinene, carene, terpineol, terpinolene, phellandrene, thujene, tricyclene, borneol, sabinene, or camphene. ispDE bifunctional enzymes described herein can be useful for generating taxol and/or taxol derivatives. ispDE bifunctional enzymes described herein can be useful for generating steroids, N-glycans, carotenoids, ubquinone, zeatin, and/or polyprenols.

In some embodiments, the bifunctional MEP pathway enzyme comprises a flexible linker peptide between an ispD domain or functional fragment thereof and an ispE domain or functional fragment thereof. In some embodiments, the flexible linker comprises the sequence of SLGGGGSAAA. In some cases, the linker sequence has a greater than 65% random coil formation as determined by GOR algorithm, version IV (Methods in Enzymology 1996 R. F. Doolittle Ed., vol 266, 540-553). In some cases, the nucleic acid encoding the ispDE protein encodes a flexible peptide linker between the ispE and ispD domains. In some cases, the flexible linker is from 6 to 15 amino acids in length. In some cases, the flexible linker is from 7 to 12 amino acids in length. In some cases, the flexible linker comprises at least 65% or at least 70% random coil formation as predicted by the GOR algorithm, version IV.

In one aspect, one or more of the bifunctional ispDE enzymes described herein can be encoded by a nucleic acid in an expression cassette, e.g., in a host cell. In some embodiments, the one or more bifunctional ispDE enzymes are heterologously expressed in a host cell. In some cases, the one or more bifunctional ispDE enzymes are co-expressed with one or more components of the MEP pathway in the same or a different expression cassette. MEP pathway components include, e.g., dxs, ispC, ispF, ispG, ispH, and idi. In some embodiments, the expression cassette comprising a promoter operably linked to a nucleic acid encoding the bifunctional ispDE enzyme further comprises one or more MEP pathway enzymes selected from the group consisting of dxs, ispC, ispF, ispG, ispH, and idi. In one embodiment, the expression cassette comprising a promoter operably linked to the bifunctional ispDE enzyme further comprises dxs, ispF and idi. In one embodiment, the expression cassette comprising a promoter operably linked to a nucleic acid encoding the bifunctional ispDE pathway enzyme further comprises a bifunctional ispDF pathway enzyme, as described in International Application No. PCT/CA2018/051074, the disclosure of which is expressly incorporated by reference herein.

In some cases, the one or more bifunctional ispDE enzymes are co-expressed with one or more aromatic prenytransferases in the same or a different expression cassette. In some cases, the one or more bifunctional ispDE enzymes are co-expressed with one or more cannabinoid synthases in the same or a different expression cassette. In some embodiments, the present invention provides an expression cassette or system of expression cassettes for heterologous expression in a host cell of a cannabinoid synthase (e.g., CBDAS or THCAS, preferably CBDAS), and the bifunctional ispDE enzyme

In some embodiments, the present invention provides an expression cassette or system of expression cassettes for heterologous expression in a host cell of one or more bifunctional ispDE enzymes, and one or more terpenoid synthases including but not limited to isoprene synthase, or lycopene synthase. In some embodiments, the expression cassette or system of expression cassettes comprise a nucleic acid encoding one or more components of a lycopene synthesis pathway (e.g., crtE, crtI, and/or crtB), a diterpene synthase, a sesquiterpene synthase, or a monoterpene synthase. In some embodiments, the expression cassette or system of expression cassettes comprise a nucleic acid encoding carene synthase, myrcene synthase, or limonene synthase. In some embodiments, the expression cassette or system of expression cassettes optionally comprises components of a lycopene synthesis pathway (e.g., crtE, crtI, and/or crtB), an isoprene synthase, a GPP synthase (e.g., ispA or a plant derived GPP synthase), a monoterpene synthase, and/or a cannabinoid synthase.

In some cases, the one or more bifunctional ispDE enzymes are co-expressed with one or more aromatic prenytransferases and one or more cannabinoid synthases (e.g., CBDAS and/or THCAS) in the same or a different expression cassette. In some embodiments, the cannabinoid synthase is selected from the group consisting of a Cannabis CBGA synthase.

The nucleic acid encoding the bifunctional ispDE can be in an MEP pathway expression cassette such as any one of the foregoing expression cassettes that contain a nucleic acid encoding an MEP pathway gene. In some cases, the nucleic acid encoding the bifunctional ispDE can be in an expression cassette that contains a nucleic acid encoding a cannabinoid synthase. In some cases, the nucleic acid encoding the bifunctional ispDE can be in an expression cassette that contains a nucleic acid encoding GPP synthase. In some cases, the nucleic acid encoding the bifunctional ispDE can be in an expression cassette that contains a nucleic acid encoding an isoprene synthase.

Methods and compositions described herein can be used for production of GPP from precursors produced in the MEP pathway in a suitable (e.g., prokaryotic) host cell, wherein the GPP is a prenyl donor substrate of the aromatic prenyltransferase and the aromatic acid is a prenyl acceptor of the aromatic prenyltransferase. Accordingly, in some embodiments, an expression cassette comprising a promoter operably linked to a nucleic acid encoding GPP synthase is provided. The GPP synthase can be in an expression cassette that also contains nucleic acid encoding a gene of the MEP pathway. Additionally, or alternatively, the GPP synthase can be in an expression cassette that also contains nucleic acid encoding a cannabinoid synthase. In some cases, the promoter of the expression cassette that is operably linked to a nucleic acid encoding GPP synthase is also operably linked to a cannabinoid synthase. Additionally, or alternatively, the GPP synthase can be in an expression cassette that also contains nucleic acid encoding an isoprene synthase.

### Host Cells

Any of the foregoing expression cassettes, and combinations thereof, can be introduced into a suitable host cell and used for production of a target metabolite, such as a cannabinoid or a prenylated aromatic acid. Suitable host cells include, but are not limited to prokaryotes, such as a prokaryote of the genus Escherichia, Panteoa, Corynebacterium, Bacillus, or Lactococcus. Preferred prokaryote host cells include, but are not limited to, Escherichia coli (E. coli), Panteoa citrea, C. glutamicum, Bacillus subtilis, and Lactococcus lactis. In some embodiments, the host cell is a eukaryotic host cell. In some embodiments, the expression cassettes described herein comprise a promoter (e.g., heterologous promoter) operably linked to a nucleic acid that encodes one or more target genes (e.g., an MFS aromatic acid antiporter (e.g., pcaK), an OMP superfamily porin, an OprD family porin (e.g., pp3656), an aromatic prenyltransferase, an MEP pathway gene, a cannabinoid synthase gene, ispA, ispS, ispDF, or GPP synthase), wherein the nucleic acid encoding the one or more target genes is codon optimized for the host cell that comprises the expression cassette.

In some cases, the host cell comprises one or more products of the MEP pathway, such as DMAPP and/or IPP. For example, a host cell containing an MEP pathway expression cassette as described herein can comprise an increased amount of an MEP pathway product such as DMAPP and/or IPP as compared to a host cell that does not contain an MEP pathway expression cassette.

In some cases, the host cell can comprise one or more products that are downstream of the MEP pathway. For example, a host cell comprising a GPP synthase expression cassette can comprise an increased amount of GPP as compared to a host cell lacking the GPP synthase expression cassette. As another example, a host cell comprising an isoprene synthase expression cassette can comprise an increased amount of isoprene as compared to a host cell lacking the isoprene synthase expression cassette.

As yet another example, a host cell comprising a cannabinoid synthase expression cassette can comprise an increased amount of cannabinoid as compared to a host cell lacking the expression cassette containing the heterologous nucleic acid encoding the heterologous transporter or functional fragment thereof. In some cases, the cannabinoid is CBGA. In some cases, the cannabinoid is CBCA. In some cases, the cannabinoid is CBDA. In some cases, the cannabinoid is THCA. In some cases, the cannabinoid is CBNA or is CBN. In some cases, the cannabinoid is CBD. In some cases, the cannabinoid is THC. In some cases, the cannabinoid is CBC. In some cases, the cannabinoid is THCV. In some cases, the cannabinoid is CBDV. In some cases, the cannabinoid is CBCV.

Similarly, the host cell can comprise an elevated amount of a product of one or more enzymes encoded by an expression cassette in the host cell when the host cell is cultured under conditions suitable to induce expression from the expression cassette as compared to non-inducing conditions. For example, the host cell can comprise an elevated intracellular amount of aromatic acid substrate of the heterologous transporter or an increased rate of intracellular accumulation of the aromatic acid substrate when induced as compared to the same host cell cultured in the absence of an inducer. As another example, the host cell can comprise an elevated amount of, or an increased rate of production of, a product of the aromatic prenyltransferase when induced as compared to the same host cell cultured in the absence of an inducer. As another example, the host cell can exhibit increased DMAPP and/or IPP when induced as compared to the same host cell cultured in the absence of an inducer (e.g., in the absence of IPTG, arabinose, etc.). As another example, the host cell can exhibit increased GPP when induced as compared to the same host cell cultured in the absence of an inducer (e.g., in the absence of IPTG, arabinose, etc.). As another example, the host cell can exhibit increased isoprene when induced as compared to the same host cell cultured in the absence of an inducer (e.g., in the absence of IPTG, arabinose, etc.). As another example, the host cell can exhibit increased cannabinoid when induced as compared to the same host cell cultured in the absence of an inducer (e.g., in the absence of IPTG, arabinose, etc.).

In some embodiments, the host cell comprises olivetolate (OA). OA can be introduced into the host cell by culturing the host cell in a medium containing OA. In some embodiments, the host cell comprises divarinic acid (DVA). DVA can be introduced into the host cell by culturing the host cell in a medium containing DVA. In typical embodiments, the OA and/or DVA are substrates of the heterologous transporter.

In some embodiments, the host cell is genetically modified to delete or reduce the expression of one or more genes that encode an endogenous enzyme that reduces flux through the MEP pathway. In some embodiments, the host cell is genetically modified to delete or reduce the amount or activity of an endogenous enzyme that reduces flux through the MEP pathway. For example, pyruvate and glyceraldehyde-3 phosphate (G3P) are the substrates of the initial enzyme of the MEP pathway dxs. Endogenous pathways that consume pyruvate and G3P can be modified to increase the amount of pyruvate and G3P thus increasing the flux through the MEP pathway. In some cases, one or more host cell endogenous genes or gene products selected from the group consisting of ackA-pta, poxB, ldhA, dld, adhE, pps, and atoDA are modified to increase pyruvate or G3P levels.

### Culture Methods

The present invention furthermore provides a process for culturing a host cell according to the present invention in a suitable medium under induction conditions, resulting in production of a target metabolic product. The target metabolic product can be a cannabinoid, a terpenoid, or a precursor thereof. The method can include concentrating the metabolite in the spent medium and/or in the host cells.

The microorganisms produced may be cultured continuously—as described, for example, in WO 05/021772—or discontinuously in a batch process (batch cultivation) or in a fed-batch or repeated fed-batch process for the purpose of producing the desired organic-chemical compound. A summary of a general nature about known cultivation methods is available in the textbook by Chmiel (BioprozeStechnik 1: Einfiihrung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)).

The culture medium or fermentation medium to be used must in a suitable manner satisfy the demands of the respective strains. Descriptions of culture media for various microorganisms are present in the “Manual of Methods for General Bacteriology” of the American Society for Bacteriology (Washington D.C., USA, 1981). The terms culture medium and fermentation medium are interchangeable.

It is possible to use, as carbon source, sugars and carbohydrates such as, for example, glucose, sucrose, lactose, fructose, maltose, molasses, sucrose-containing solutions from sugar beet or sugar cane processing, starch, starch hydrolysate, and cellulose; oils and fats such as, for example, soybean oil, sunflower oil, groundnut oil and coconut fat; fatty acids such as, for example, palmitic acid, stearic acid, and linoleic acid; alcohols such as, for example, glycerol, methanol, and ethanol; and organic acids such as, for example, acetic acid or lactic acid.

It is possible to use, as nitrogen source, organic nitrogen-containing compounds such as peptones, yeast extract, meat extract, malt extract, corn steep liquor, soybean flour, and urea; or inorganic compounds such as ammonium sulfate, ammonium chloride, ammonium phosphate, ammonium carbonate, and ammonium nitrate. The nitrogen sources can be used individually or as a mixture.

It is possible to use, as phosphorus source, phosphoric acid, potassium dihydrogen phosphate or dipotassium hydrogen phosphate or the corresponding sodium-containing salts.

The culture medium may additionally comprise salts, for example in the form of chlorides or sulfates of metals such as, for example, sodium, potassium, magnesium, calcium and iron, such as, for example, magnesium sulfate or iron sulfate, which are necessary for growth. Finally, essential growth factors such as amino acids, for example homoserine and vitamins, for example thiamine, biotin or pantothenic acid, may be employed in addition to the abovementioned substances.

Said starting materials may be added to the culture in the form of a single batch or be fed in during the cultivation in a suitable manner.

The pH of the culture can be controlled by employing basic compounds such as sodium hydroxide, potassium hydroxide, ammonia, or aqueous ammonia; or acidic compounds such as phosphoric acid or sulfuric acid in a suitable manner. The pH is generally adjusted to a value of from 6.0 to 8.5, preferably 6.5 to 8. To control foaming, it is possible to employ antifoams such as, for example, fatty acid polyglycol esters. To maintain the stability of plasmids, it is possible to add to the medium suitable selective substances such as, for example, antibiotics. The culturing is preferably carried out under aerobic conditions. In order to maintain these conditions, oxygen or oxygen-containing gas mixtures such as, for example, air are introduced into the culture. It is likewise possible to use liquids enriched with hydrogen peroxide. The culturing is carried out, where appropriate, at elevated pressure, for example at an elevated pressure of from 0.03 to 0.2 MP a. The temperature of the culture is normally from 20° C. to 45° C. and preferably from 25° C. to 40° C., particularly preferably from 30° C. to 37° C. In batch or fed-batch processes, the cultivation is preferably continued until an amount of the desired organic-chemical compound sufficient for being recovered has formed. This aim is normally achieved within 10 hours to 160 hours (e.g., within 10 to 72 hours, 10 to 48 hours, 10-24 hours, or 10-16 hours). In continuous processes, longer cultivation times are possible. The activity of the microorganisms results in a concentration (accumulation) of the organic-chemical compound in the fermentation medium and/or in the cells of said microorganisms.

Examples of suitable culture media can be found inter alia in the U.S. Pat. Nos. 5,770,409, 5,990,350, 5,275,940, WO 2007/012078, U.S. Pat. No. 5,827,698, WO 2009/043803, U.S. Pat. Nos. 5,756,345 and 7,138,266.

Analysis of target metabolic products to determine the concentration at one or more time(s) during the culturing can take place by separating the metabolites by means of chromatography, preferably reverse-phase chromatography.

Detection can be carried out carried out photometrically (absorption, fluorescence).

The performance of the culture methods using a host cell containing one or more expression cassettes according to the invention, in terms of one or more of the parameters selected from the group of concentration (target metabolic product formed per unit volume), yield (target metabolic product formed per unit carbon source consumed), formation (target metabolic product formed per unit volume and time) and specific formation (target metabolic product per unit dry cell matter or dry biomass and time or compound formed per unit cellular protein and time) or else other process parameters and combinations thereof, can be increased by at least 0.5%, at least 1%, at least 1.5%, at least 2%, at least 3%, at least 4%, at least 5%, at least 10%, at least 20%, at least 30%, at least 40%, at least 50%, at least 60%, at least 70%, at least 80%, at least 90%, or at least 100% based on culture methods using host cells that do not contain the expression cassettes according to the invention. This is considered to be very worthwhile in terms of a large-scale industrial process.

A product containing the target metabolic product can then be provided or produced or recovered in liquid or solid form.

Spent medium means a culture medium in which a host cell has been cultured for a certain time and at a certain temperature. The culture medium or the media employed during culturing comprise(s) all the substances or components which ensure production of the desired target metabolic product and typically propagation and viability. When the culturing is complete, the resulting spent medium accordingly comprises: a) the biomass (cell mass) of the microorganism, said biomass having been produced due to propagation of the cells of said microorganism; b) the desired target metabolic product formed during the culturing; c) the organic byproducts possibly formed during the culturing; and d) the constituents of the culture medium employed or of the starting materials, such as, for example, vitamins such as biotin or salts such as magnesium sulfate, which have not been consumed in the culturing.

The organic byproducts include substances which are produced by the microorganisms employed in the culturing in addition to the particular desired compound and are optionally secreted. The spent medium can be removed from the culture vessel or fermentation tank, collected where appropriate, and used for providing a product containing the target metabolic product in liquid or solid form. In the simplest case, the target metabolic product-containing spent medium itself, which has been removed from the fermentation tank, constitutes the recovered product.

In some cases, recovering the target metabolic product (e.g., terpenoid, cannabinoid, or precursor thereof) includes, but is not limited to, one or more of the measures selected from the group consisting of a) partial (>0% to <80%) to complete (100%) or virtually complete (>80%, >90%, >95%, >96%, >97%, >98%, or >99%) removal of the water; b) partial (>0% to <80%) to complete (100%) or virtually complete (>80%, >90%, >95%, >96%, >97%, >98%, or >99%) removal of the biomass, the latter being optionally inactivated before removal; c) partial (>0% to <80%) to complete (100%) or virtually complete (>80%, >90%, >95%, >96%, >97%, >98%, >99%, >99.3%, or >99.7%) removal of the organic byproducts formed during culturing; and d) partial (>0%) to complete (100%) or virtually complete (>80%, >90%, >95%, >96%, >97%, >98%, >99%, >99.3%, or >99.7%) removal of the constituents of the fermentation medium employed or of the starting materials, which have not been consumed in the culturing, from the spent medium achieves concentration or purification of the desired target metabolic product. In some cases, the target metabolic product is produced intracellularly and recovered by a method including lysis of cultured host cells of the invention. In some cases, a method of recovering target metabolic product includes providing lysate of a cultured host cell of the invention and isolating the target metabolic product from the lysate. Compositions having a desired content of said target metabolic product are thereby isolated. Lysing of cultured host cells can be performed, e.g., after isolating host cells from spent media.

The partial (>0% to <80%) to complete (100%) or virtually complete (>80% to <100%) removal of the water (measure a)) is also referred to as drying.

In one variant of the process, complete or virtually complete removal of the water, of the biomass, of the organic byproducts and of the unconsumed constituents of the fermentation medium employed results in pure (>80% by weight, >90% by weight) or high-purity (>95% by weight, >97% by weight, or >99% by weight) product forms of the desired target metabolic product. An abundance of technical instructions for measure a) is available in the prior art.

Depending on requirements, the biomass can be removed wholly or partly from the spent medium by separation methods such as, for example, centrifugation, filtration, decantation or a combination thereof, or be left completely therein. Where appropriate, the biomass or the biomass-containing spent medium is inactivated during a suitable process step, for example by thermal treatment (heating) or by addition of alkaline or acid.

In one procedure, the biomass is completely or virtually completely removed so that no (0%) or at most 30%, at most 20%, at most 10%, at most 5%, at most 1% or at most 0.1% biomass remains in the prepared product. In a further procedure, the biomass is not removed, or is removed only in small proportions, so that all (100%) or more than 70%, 80%, 90%, 95%, 99% or 99.9% biomass remains in the product prepared. In one process according to the invention, accordingly, the biomass is removed in proportions of from >0% to <100%. Finally, the fermentation broth obtained after the fermentation can be adjusted, before or after the complete or partial removal of the biomass, to an acidic pH with an inorganic acid such as, for example, hydrochloric acid, sulfuric acid, or phosphoric acid; or organic acid such as, for example, propionic acid, so as to improve the handling properties of the final product (see, e.g., GB 1,439,728 or EP 1 331220). It is likewise possible to acidify the fermentation broth with the complete content of biomass. Finally, the broth can also be stabilized by adding sodium bisulfite (NaHCO₃, GB 1,439,728) or another salt, for example ammonium, alkali metal, or alkaline earth metal salt of sulfurous acid.

During the removal of the biomass, any organic or inorganic solids present in the spent medium can be partially or completely removed. The organic byproducts dissolved in the spent medium, and the dissolved unconsumed constituents of the fermentation medium (starting materials), can remain at least partly (>0%), in some cases to an extent of at least 25%, in some cases to an extent of at least 50% and in some cases to an extent of at least 75% in the product. Where appropriate, they also remain completely (100%) or virtually completely, meaning >95% or >98% or >99%, in the product.

Subsequently, water can be removed from the spent medium, or said spent medium can be thickened or concentrated, by known methods such as, for example, using a rotary evaporator, thin-film evaporator, falling-film evaporator, by reverse osmosis or by nanofiltration. This concentrated spent medium can then be worked up to free-flowing products, in particular to a fine powder or preferably coarse granules, by methods of freeze drying, spray drying, spray granulation or by other processes such as in the circulating fluidized bed, as described for example according to PCT/EP2004/006655.

### REFERENCES

The following publications are incorporated herein by this reference. These publications are referred to herein by the numbers provided below. The inclusion of any publication in this list of publications is not to be taken as an admission that any publication referred to herein is prior art.
JAMA. 2006; 295(7): 761-775
Comput Struct Biotechnol J, 2012, 3, 1-11
Biotechnol. Bioeng. 2004 88, 909-915.
Science 2002, 298 (5599), 1790-3.
Sonal R. Ayakar (2019), Biocatalysis and bioprocess engineering for terpenoid production, PhD thesis, University of British Columbia, Canada

### Examples

### Example 1: Aromatic Prenyltransferase Substrate Transporter Expression in E. coli

### Cloning:

Two different transporters PcaK and PP 3656 were amplified from Pseudomonas putida KT2440 by PCR and were cloned into a plasmid under pTrc promoter. This plasmid was then transformed into BL21 DE3 for its expression and used for the transporting the aromatic compound into the BL21 DE3.

### Making Seed Culture:

The single colony was picked from the agar plate, streaked previously from the glycerol stock (of BL21 DE3, and BL21 DE3 cells containing plasmid pTrc-PcaKor pTrc-PP3656) and grown into LB media (5 ml) with 100 μg/ml carbenicillin (for overnight BL21DE3 containing plasmid) [typically 16 hrs] at 37° C.

### Inoculation, Induction and Expression:

Seed culture from overnight was inoculated into fresh 5 ml LB media at the OD600=0.1 and was allowed to grow at 37° C. until the OD600 reaches to 0.6. [typically, it takes 2.5 to 3 hrs]. The cell culture was induced with 100 μM IPTG in case of BL21 DE3 containing plasmid. Both the cells were then fed with 0.1 mM olivetolate and were allowed to grow 6 hours, 24 hours and 48 hours at 30° C. and/or 22° C.

### Harvesting:

The cells were then harvested [typically, after 14 to 16 hrs] by centrifuging the overnight culture at 3500 rpm for 20 min. The cell pellet was used to lyse or kept at −80° C. for overnight to store. The supernatant was stored at −20° C. for HPLC analysis (supernatant 1).

### Lysing the Cell:

The cells were lysed by resuspending the entire pellet from a 5 mL culture in to 300 lysis buffer (lysis buffer composition: 50 mM Tris pH 8, 10% glycerol, 0.1% Triton X 100, 100 μg/ml lysozyme, 1 mM PMSF, DNAse 3U, 2 mM MgCl₂) and afterwards sonicating cell pellet using probe sonicator. The cell pellet suspended in lysis buffer was always maintained on ice during cell lysis and sonication was done (in cycle of 15 sec pulse and 30 sec rest on ice) for 10 cycles. After the lysis, crude cell lysate supernatant was collected by centrifugation at 14000 rpm for 20 min at 4° C. The supernatant was used for HPLC analysis or stored in −80° C. (supernatant 2).

### HPLC Analysis:

Supernatant 1 was filtered through a 0.1 μm filter and 300 μL of filtrate was used for HPLC analysis. Supernatant 2 was centrifuged at 14000 rpm for 10 min and 300 μL of the upper clear supernatant was used for HPLC analysis. HPLC analysis was performed on Perkin Elmer HPLC equipped with Flexar PDA plus multi wavelength detector and Chromera software. The conditions for HPLC analysis are as follows:
HPLC column: LUNA OMEGA 3 μm Polar C18 Column (150×4.6 mm)
Mobile Phase: 75% ACN, 25% water, 0.1% formic acid
Flow rate: 1 ml/min
Detection wavelength: 230 and 270 nm
Oven temp: 25° C.
Injection volume: 10 μL
Run time: 18 min

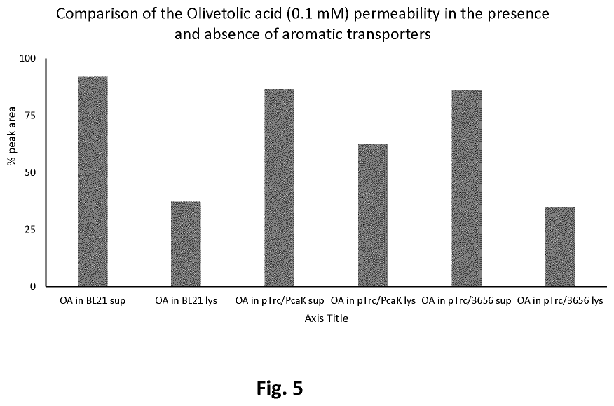

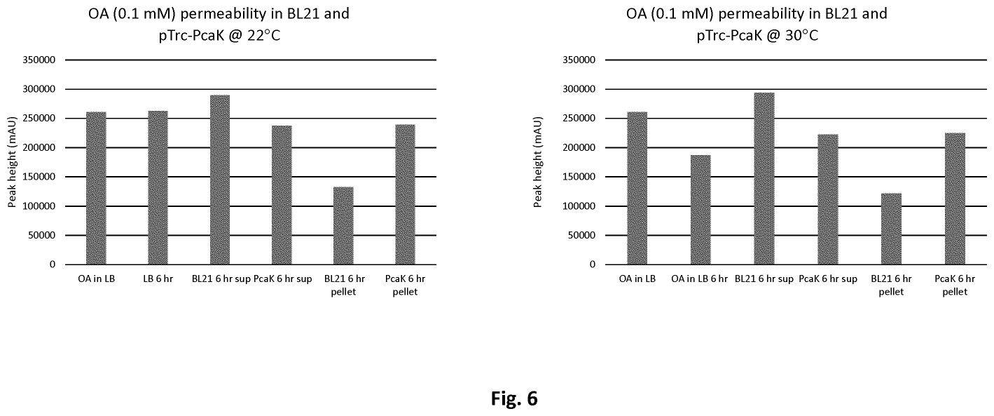

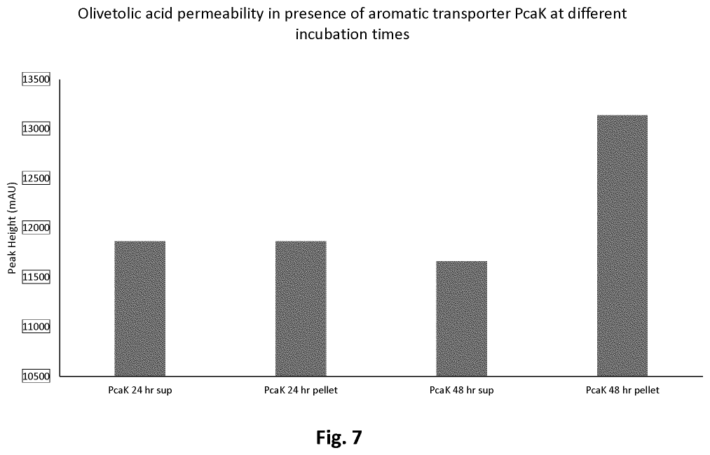

Results are depicted in FIGS. 5-7.

### Example 2: Aromatic Prenyltransferase Substrate Transporter Expression and Cannabinoid Production in E. coli

### Making Seed Culture:

The following experimental host cells were tested: (1) E. coli transformed with plasmids encoding arabinose inducible transporters pcaK or pp3656; and (2)) E. coli transformed with plasmids encoding arabinose inducible transporters pcaK or pp3656 and B5 plasmid (encoding ispDF₁ enzyme, GPPS, and an optimized variant NphB (see, Valliere et al.).

Seed cultures of (1) were inoculated from glycerol stock into 5 mL LB with 34 μg/mL chloramphenicol, incubate at 30° C. overnight. Seed cultures of (2) were inoculated from glycerol stock into 5 mL LB with 34 μg/mL chloramphenicol and 50 μg/mL kanamycin, incubate at 30° C. overnight.

### Inoculation, Induction and Expression:

Induction cultures of (1) were inoculated from seed culture into total culture volume 5 mL TB media with 0.1 mM OA and cultured at 30° C. until OD600 of 0.8. Cultures were induced by adding arabinose and magnesium to a final concentration of 5 mM arabinose and 5 mM MgCl₂. During induction, cultures were incubated at 30° C. Induction culture samples were collected at 24 h and 48 hr time points after the start of induction.

Induction cultures of (2) were inoculated from seed culture into total culture volume 5 mL TB media with 0.5 mM OA, 5 mM MgCl₂, and cultured at 30° C. until OD600 of 0.8. Cultures were induced by adding arabinose to a final concentration of 5 mM arabinose and IPTG to a final concentration of 100 μM. During induction, cultures were incubated at 30° C. Induction culture samples were collected at 24 h and 48 hr time points after the start of induction.

### Extraction of OA or CBGA:

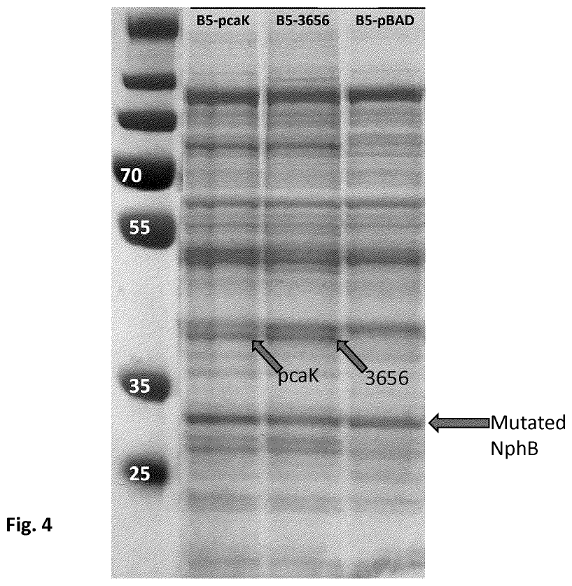

Cultures first centrifuged at 3000 rpm for 10 m to separate pellet and culture media supernatant fractions. Pellets also washed with PBS twice. Cell pellets lysed with B-PER Complete Reagent following manufacturer's protocol. Briefly, pellets were resuspended in B-PER, incubated at 25° C. for 20 m, and insoluble material was centrifuged down at 14000 rpm for 20 m. The soluble material was preserved as a cell lysate. Samples of the cell lysates were analyzed by SDS-PAGE analysis. See, FIG. 4.

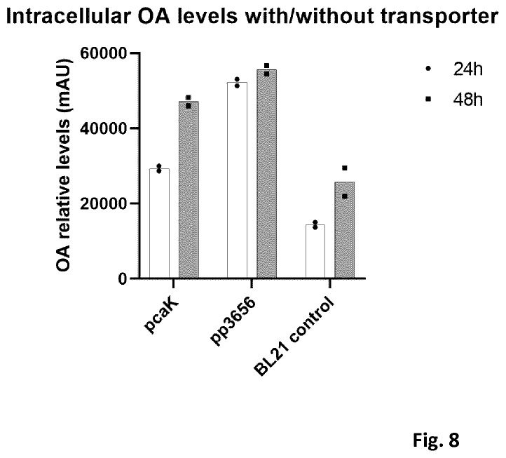

To extract OA from the cell lysates, ethyl acetate was added to the soluble lysate fraction at 1:1 volume ratio and vigorously mixed. Organic and aqueous fractions were separated by centrifuging at 14000 rpm for 20 m. Organic phase was evaporated away using a speed vacuum and resuspended in HPLC mobile phase (75% ACN, 25% water, 0.1% formic acid) for analysis. Analysis results are depicted in FIG. 8.

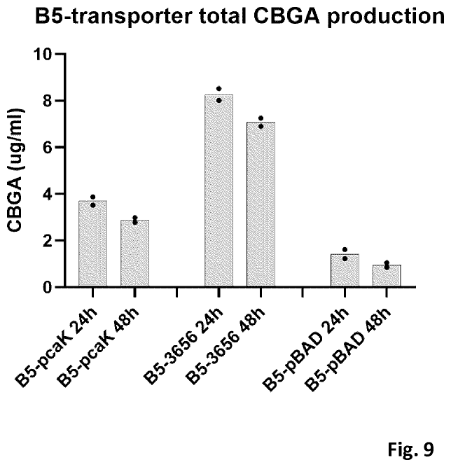

To extract CBGA from the cell lysates, ethyl acetate was added to culture media supernatant at 1:1 volume ratio and vigorously mixed. Organic and aqueous fractions were separated by centrifuging at 14000 rpm for 20 m. Organic phase was evaporated away using a speed vacuum and resuspended in HPLC mobile phase (75% ACN, 25% water, 0.1% formic acid) for analysis. Analysis results are depicted in FIG. 9.

### Conclusion:

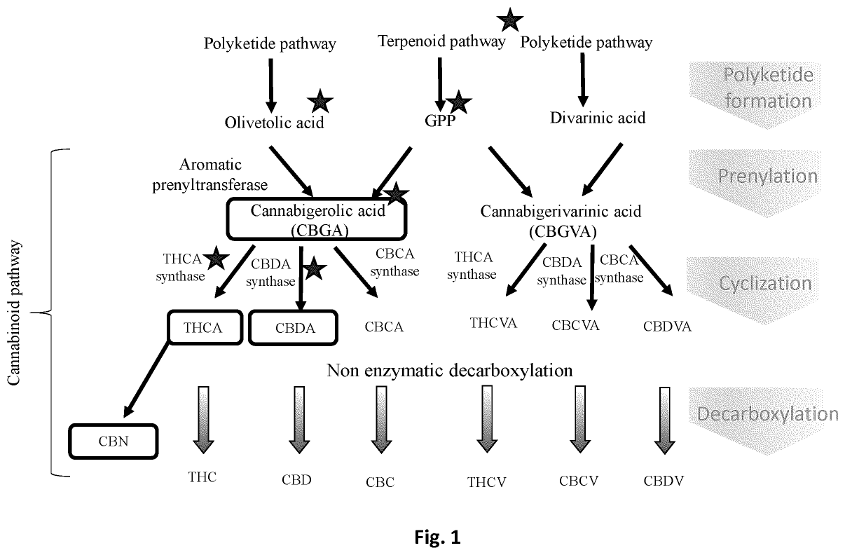

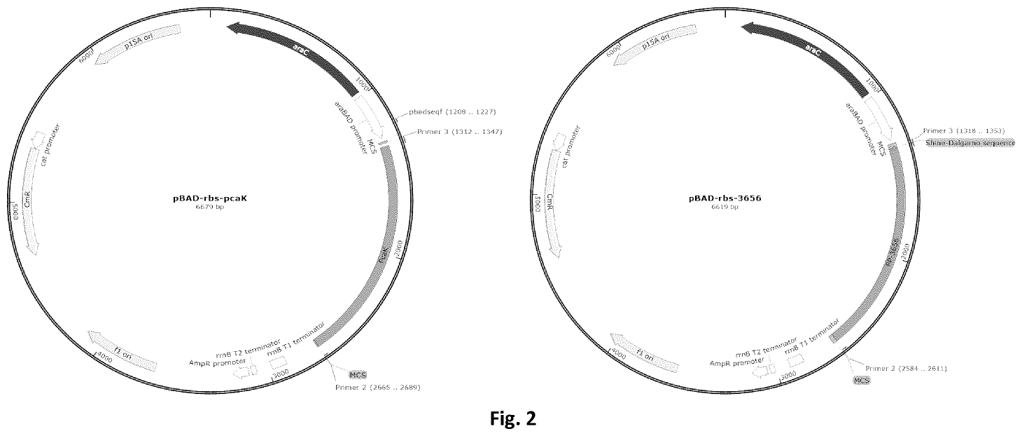

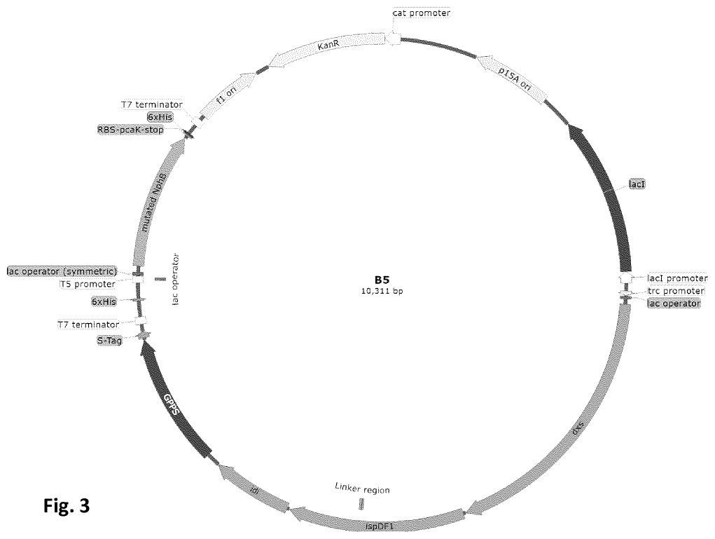

Host cells expressing a heterologous aromatic prenyltransferase and a transporter capable of transporting a substrate of the aromatic prenyltransferase (e.g., olivetolate) into the cell exhibit increased production of one or more products of the aromatic prenyltransferase enzyme when cultured in media containing exogenously applied aromatic prenyltransferase substrate (e.g., olivetolate). See. FIGS. 1 to 4 and 8 to 9.

### Example 3: ispDE Expression and Analysis

### Introduction

Flux through MEP pathway in E. coli is very low though disruption of the pathway genes was reported to be lethal in E. coli^{63,64}. The pathway downstream to Dxs catalytic step can be complemented with heterologous expression of rate determining enzymes of the MVA pathway⁶⁵. Dxs deletion cannot be complemented with MVA pathway because of its role in vitamin B6 and Bi biosynthesis³⁰. Whereas IPP and DMAPP are essential for prenylation of t-RNAs⁶⁶ and quinones⁶⁷.

As discussed herein, MEP operates at a higher theoretical yield and is thermodynamically favored over MVA pathway²³. The experimentally observed MEP pathway yield is far from the theoretical maxima. MEP pathway can be used to generate a most robust heterologous platform for isoprenoid biosynthesis on optimization.

### Improvements in the Precursor Supply for the MEP Pathway

GAP and pyruvate are the metabolites from the glycolytic pathway involved in central carbon metabolism. Efforts of improving flux through glycolysis have been limited by the attempts at enhancing sugar uptake rate⁶⁸⁻⁷⁰. As the glucose transporter was made more active, various steps in the glycolytic pathway lost their metabolic control⁷¹. The thermodynamics of conversion of fructose-1,6-diphosphate to DHAP and GAP push the equilibrium towards the substrate⁷². Isomerization of DHAP and GAP is favored towards DHAP. Some successful efforts have been to channel the flux through the pentose phosphate pathway and ED pathway for isopentenol production⁷³. The distribution between GAP and pyruvate has a role in driving flux through the MEP pathway and redirection of flux to GAP from pyruvate lead to improvement in downstream lycopene production⁷⁴. The same study also reported that feeding GAP and pyruvate does not change the flux substantially.

### MEP Pathway Optimization

Improvements in genome sequencing, genome mining, proteomics, metabolomics and bioinformatic tools have provided the field of metabolic engineering to find wider applications.

A well-studied strategy is an optimization through tools of metabolic engineering. Heterologous overexpression of homologous MEP pathway bottlenecks have proven to greatly enhance synthesis of terminal isoprenoid products. Overexpression of four genes—dxs, ispD, ispF and idi were shown to improve taxol yield in E. coli²⁴. Whereas, overexpression of dxs, ispD, ispF and ispH improved lycopene yield by 15-fold in Bacillus subtilis⁷⁵.

MEP flux can be upregulated by expression of higher active heterologous MEP pathway enzymes. This involves the replacement of a single enzyme or the entire pathway chassis. Dxs from Arabidopsis thaliana was expressed in transgenic Lavandula latifolia led to a 5-fold higher total terpenoid yield⁷⁶.

The genes involved in the MEP pathway are controlled by constitutive promoters. Chromosomal exchange of dxs promoter with a strong promoter P_{tuf} in Corynebacterium glutamicum achieved 60% improved Dxs activity and doubled lycopene production⁴⁷.

Reasons for flux limitations lie in one or more of these factors: low activity, low stability, low expression levels, low solubility, feedback regulation or toxicity. The strategy of modification of these enzymes at genetic levels through mutation has been tried. Directed co-evolution of Dxs, Dxr and Idi lead to 60% improvement in lycopene yield in E. coli⁷⁷.

Dxs, IspG, IspH and IDI suffer from low solubility and form inactive inclusion bodies on overexpression. Improvement in their solubility will lead to enhanced activity. Lowering incubation temperature, co-expression with chaperone proteins and protein mutagenesis improve the solubility of the otherwise insoluble protein. Another strategy of supplementing growth media with betaine and sorbitol increased the Dxs solubility by 60%. This also led to overall improvement in the MEP pathway flux⁷⁸.

The occurrence of fused IspDF enzyme is common in α and ε proteobacterial genomes but not so in β and γ proteobacterial genomes⁷⁹. IspDF is isolated and studied in detail from Campylobacter jejuni⁷⁹, Mesorhizobium loti⁸⁰ and Agrobacterium tumefaciens⁸¹.

The first bifunctional gene was isolated from Campylobacter jejuni⁷⁹, a product of which (cjIspDF, 42 kDa polypeptide) catalyzed two reactions individually carried out by IspD and IspF with rates of 3.9 μmol·mg⁻¹·min⁻¹ and 0.8 μmol·mg⁻¹·min⁻¹ respectively. The cjIspDF had a greater similarity with E. coli IspF (approx. 48%) than ispD (approx. 25%). In vitro reactions with purified His tagged protein from recombinant E. coli employing ¹³C labeled MEP yielded CDP-ME and addition of Zn⁺² ion as cofactor gave highest rate (18.5 μmol·mg⁻¹·min⁻¹) with Km values of 3 μM and 20 μM for CTP and MEP respectively at pH 5. Presence of ATP did not alter the reaction kinetics until IspE was added when it led to the formation of MEcPP with the highest activity at pH 8 and Ca⁺² as a cofactor with 19 μM Km value for CDP-MEP. The estimated shortest distance between the two catalytic centers of IspD and IspF subunits in the cjIspDF is around 38 Å. The cjIspDF was reported to exist as a trimer, hexamer and dodecamer when analyzed by size exclusion chromatography⁷⁹ whereas, the crystal structure is hexameric⁶². It also shows two clear domains for each of the domains joined by a linker sequence. The hexameric assembly contains two trimers of IspD domain dimers and two trimers of IspF domain trimers. In this hexameric complex, one of the IspF domains of corresponding dimers IspD domains associate to form trimers. This means that the individual domains of the same bifunctional polypeptide do not associate.

Another well studied bifunctional IspDF from Mesorhizobium loti (mlIspDF) was expressed in E. coli and was also found to exhibit catalytic activities of both IspD and IspF⁸⁰. The IspD subunit had 46% similarity with E. coli IspD whereas, The IspF subunit had 44% similarity with E. coli IspF. Size exclusion chromatography of the protein sample showed the existence of monomeric unit and dimeric complex of mlIspDF. Higher molecular complexes were not observed.

Experiments on monomeric E. coli enzymes were performed and analyzed by sedimentation velocity method for 3 sets of combinations: (a) IspD and IspE, (b) IspE and IspF; and (c) IspD, IspE and IspF. These studies revealed the assembly of three IspD dimers, three IspE dimers, and two IspF trimers⁶². The same study revealed that the domains IspD and IspF from IspDF associate with IspE to form a mega complex⁶² and aid the substrate channeling. This was reported for both cjIspDF and atIspDF⁸¹ (IspDF from Agrobacterium tumefaciens) Trimers of IspD dimer and IspE dimer complex with dimers of IspF trimers to form an assembly of 18 catalytic centers. atIspDF was also detected to associate at higher molecular weight ratios. For cjIspDF, the distance between the two catalytic centers of the same multimer is 35 Å for IspD subunit and 30 Å for IspF subunit which is lesser than the distance between the two catalytic centers of the cjIspDF.

On the other hand, a similar study⁸¹ was done on IspDF and IspE isolated from Agrobacterium tumefaciens (atIspDF and atIspE respectively). These enzymes were not found to associate based on sedimentation velocity experiments. Further validation was confirmed in vitro condition by adding an inactive form of atIspE by A152A point mutation. The inactive IspE did not change the reaction course of conversion of MEP to MEcPP through atIspDF and atIspE cascade. The mutated IspE should have interacted with the complex and lowered the overall rate of reaction if the enzymes associate to facilitate substrate channeling. The other examples of fusions where the active sites but do not channel the substrates. GlmU enzyme from E. coli involved in peptidoglycan biosynthesis is a bifunctional enzyme that catalyzes the consecutive steps in the pathway but the intermediate is released from the first active site, accumulates in the environment to be acted upon by the second functionality⁸².

Natural occurrence of fusions enzymes that catalyze non-consecutive steps in a biosynthetic pathway is rare²¹. Gram-positive bacteria like Enterococcus faecalis and Enterococcus faecium encode a bifunctional enzyme MvaE that possesses both 3-hydroxy-3-methylglutaryl CoA (HMG-CoA) reductase and acetyl-CoA acetyltransferase activities that are involved in MVA pathway and are separated by one step catalyzed by HMG-CoA synthase^{83,84}. But no association complex is reported. The second example is involved in the carotenoid biosynthetic pathway. The carRA gene identified in fungi—Phycomyces blakesleeanus and Mucor circinelloides that encodes fusion for phytoene synthase and lycopene cyclase^{85,86}. Phytoene synthase is a prenyl transferase that catalyzes the synthesis of phytoene (GGPP) from the condensation of two GPP molecules. Phytoene is then converted into lycopene by the dehydrogenase encoded by CarB. β-Carotene is then synthesized by cyclization catalyzed by lycopene cyclase. The reports accept the presence of exceptions of these fusions, but they fail to justify the reason as well as indicate any utility of these fusions.

The occurrence of enzyme fusions at the genetic level is common Fatty acid synthesis, polyketide synthesis pathways involve bifunctional enzymes but all of them catalyze consecutive steps in the pathway. The reasons behind the existence of the fusions like IspDF, MvaE and CraAR remain unclear. Though some researchers argue their relevance at metabolic control levels.

There lies a gap between theoretical maximum and experimentally feasible yield of the MEP pathway. Many efforts are done in the area of genome engineering, protein engineering and metabolic engineering to fill in the gap. A strategy that involves replacing the bottleneck steps with more active and/or stable orthologous enzymes has not witnessed widespread adoption. The bifunctional enzymes that are reported to be involved in the pathway are promising targets. There are no reports of influence on in vivo MEP flux by these bifunctional IspDFs. The efforts have been directed towards studying the purified proteins for their in vitro activities.

In this work we conducted metagenomic screening for identification of fusions of enzymes of the MEP pathway with consideration to enhance substrate channeling. All the fusions discovered were of IspD and IspF. These enzymes are reported to catalyze non-consecutive steps in the MEP pathway. We conducted a thorough study on the linker characteristics and their influence on MEP pathway flux. The linker sequence that connects the two domains in a bifunctional enzyme can alter enzyme activity^{87,88}. The flexibility and rigidity of the linker play a role in maintaining independence in the movements of the domains. We non-naturally fused IspE to each of IspD and IspF to mimic natural fusions. Such a robust and high yielding MEP pathway platform strain can thus be utilized to produce isoprenoids as well as to mine new compounds.

Synthetic fusion proteins that have more than one catalytic activity are designed either to expand the catalytic spectra of the protein or to improve the catalytic efficiency. Expressing a single fusion protein also substantially reduce production cost leading to higher industrial applicability⁸⁹. Chemical catalysis has widely accepted the strategy of multifunctional catalyst that is tailored to catalyze more than one type of reactions and has gained popularity in the industry^{90,91}.

There are two major ways for generating non-natural fusions⁹². First is at the genetic level by replacing transcriptional stop codon of the first gene and transcriptional start codon of the second gene with a nucleotide sequence that will generate a peptide bond on translation. The second is introducing tags in the protein that trigger an association reaction forming the peptide bond at the post-translational step.

Conversion of L-erythrulose from 2-amino-1,2,3-butanetriol was catalyzed by a novel enzyme, w-transaminase using serine as amine donor. This reaction generated hydroxypyruvate as a byproduct that was shuttled back into a substrate re-generating system as an amine donor by the action of a transketolase enzyme for the conversion of glycoaldehyde to L-erythrulose. The fusion of transaminase and transketolase created an efficient closed loop system⁹³. Another study combined four heterologously expressed enzymes to create a multienzyme reaction cascade in E. coli for the conversion of ethylbenzenes to enantiopure (R)-1-phenylethanamines eliminating the need for use of additional co-factors⁹⁴.

There are no reports on non-natural MEP pathway enzyme fusions. Absence and presence of fusions to aid active site colocalization and thereby channeling substrate for efficient conversion are highly debated topics in the field. Moreover, the fusions of IspD and IspF occur that catalyze non-consecutive steps in the pathway and fusions of IspE have been never reported.

Soil samples were collected at the Skulow Lake site (SBS-3 WL) located at coordinates 52° 20′N, 121° 55′W as a part of Long-term Soil Productivity (LTSP) study⁹⁵. High molecular weight genomic DNA was extracted and purified to create large insert fosmid NR fosmid library was created using the CopyControl™ Fosmid Library Production Kit (Epicentre) according to the manufacturer's protocol from Bt soil horizon in a naturally disturbed reference site. Twenty 384-plates from the library were Sanger end-sequenced at the Michael Smith Genome Science Center (GSC), UBC with the pCC1-Forward (5′-GGATGTGCTGCAAGGCGATTAAGTTGG) and pCC1-Reverse (5′-CTCGTATGTTGTGTGGAATTGTGAGC) primers generating 7680 paired-end sequences.

Approximately 530 fosmids were selected in silico based on phylogenetic gene markers located on the fosmid ends and functional screens and have been full-length sequenced on the Illumina HiSeq platform at the GSC. Sequence analysis including open reading frame (ORF) prediction and annotation was performed using the MetaPathways pipeline v2.5 supplied with a collection of reference databases (KEGG 2011-06-18, COG 2013-12-27, RefSeq 2014-01-18 and MetaCyc 2011-07-03)⁹⁵. Protein family searches using the online HMMER tool version 2.17.3⁹⁹ were performed to confirm functional annotations generated by the MetaPathways tool. The resulting MetaPathways outputs for the fosmid ends and fully sequenced fosmids were searched for Enzyme Commission (EC) numbers of genes encoding bifunctional ispDF. Cognate nucleotide sequences were searched against NCBI database using the online BLASTN search tool and resulting text files were uploaded into Megan 6.10.0 to assign taxonomy using the LCA algorithm⁹⁵. Based on this analysis fosmid sequences of NR0032_N05, NR0032_007 and NR0037_N05 were assigned to Acidobacteria and the ispDFs were annotated as ispDF₁, ispDF₂ and ispDF₃ respectively.

All strains, plasmids and genes used in this study are listed in Table 2.1. It contains genetic chassis with natural monomeric enzymes as well as natural fusion enzymes of the MEP pathway. Genes dxs, ispD, ispE, ispF, idi were amplified from E. coli strain K12 genome by polymerase chain reaction. Bifunctional genes ispDF1, ispDF2 and ispDF3; and ispS were codon optimized and synthesized from Genewiz Inc. pTrc-trGPPS(CO)-LS was a gift from Jay Keasling (Addgene plasmid 50603)¹⁰⁰ from where the vector backbone was amplified to construct the plasmid variants. E. coli DH5a was used as cloning host and E. coli BL21(DE3) was used as an expression host.

TABLE 2.1.
Strains, genes and plasmids used for MEP pathway study

| Strains | Description | Source |
|---|---|---|
| E. coli DH5α | Cloning strain | NEB (#C2987) |
| E. coli BL21(DE3) | Expression strain | NEB (#C2527) |
| E. coli strain K12 | Gene amplification | Sigma-Aldrich |
| | | (#EC1) |
| SASDFI | pSASDFI and expressed in E. coli BL21(DE3) | This study |
| SAIso | pSAIspS expressed in E. coli BL21(DE3) | This study |
| SAIso-SDFI | pSASDFI and pSAIspS coexpressed in E. coli BL21(DE3) | This study |
| SAIso-SDF₁I | pSASDF₁I and pSAIspS coexpressed in E. coli BL21(DE3) | This study |
| SAIso-SDF₂I | pSASDF₂I and pSAIspS coexpressed in E. coli BL21(DE3) | This study |
| SAIso-SDF₃I | pSASDF₃I and pSAIspS coexpressed in E. coli BL21(DE3) | This study |
| SAIso-SI | pSASI and pSAIspS coexpressed in E. coli BL21(DE3) | This study |
| SAIso-DF₁ | pSADF₁ and pSAIspS coexpressed in E. coli BL21(DE3) | This study |
| SAIso-DF₂ | pSADF₂ and pSAIspS coexpressed in E. coli BL21(DE3) | This study |
| SAIso-DF₃ | pSADF₃ and pSAIspS coexpressed in E. coli BL21(DE3) | This study |
| SALyc | pAC-LYC expressed in E. coli BL21(DE3) | This study |
| SALyc-SDFI | pSASDFI and pAC-LYC coexpressed in E. coli BL21(DE3) | This study |
| SALyc-SDFEI | pSASDFEI and pAC-LYC coexpressed in E. coli BL21(DE3) | This study |
| SALyc-SDF₁I | pSASDF₁I and pAC-LYC coexpressed in E. coli BL21(DE3) | This study |
| SALyc-SDF₁EI | pSASDF₁EI and pAC-LYC coexpressed in E. coli BL21(DE3) | This study |
| SALyc-SDF₂I | pSASDF₂I and pAC-LYC coexpressed in E. coli BL21(DE3) | This study |
| SALyc-SDF₃I | pSASDF₃I and pAC-LYC coexpressed in E. coli BL21(DE3) | This study |
| SALyc-SI | pSASI and pAC-LYC coexpressed in E. coli BL21(DE3) | This study |
| SALyc-DF₁ | pSADF₁ and pAC-LYC coexpressed in E. coli BL21(DE3) | This study |
| SALyc-DF₂ | pSADF₂ and pAC-LYC coexpressed in E. coli BL21(DE3) | This study |
| SALyc-DF₃ | pSADF₃ and pAC-LYC coexpressed in E. coli BL21(DE3) | This study |
| Plasmids | Description | Source |
| pSASDFI | Amp^{r}; trc promoter; genes dxs, ispD, ispF and idi; pBR322 ori | This study |
| pSASDFEI | Amp^{r}; trc promoter; genes dxs, ispD, ispF, idi and ispE; | This study |
| | pBR322 ori | |
| pSASDF₁I | Amp^{r}; trc promoter; genes dxs, ispDF₁ and idi; pBR322 on | This study |
| pSASDF₂I | Amp^{r}; trc promoter; genes dxs, ispDF₂ and idi; pBR322 on | This study |
| pSASDF₃I | Amp^{r}; trc promoter; genes dxs, ispDF₃ and idi; pBR322 on | This study |
| pSASDF₁EI | Amp^{r}; trc promoter; genes dxs, ispDF₁, idi and ispE; pBR322 | This study |
| | ori | |
| pSADF₁ | Amp^{r}; trc promoter; ispDF₁; pBR322 ori | This study |
| pSADF₂ | Amp^{r}; trc promoter; ispDF₂; pBR322 ori | This study |
| pSADF₃ | Amp^{r}; trc promoter; ispDF₃; pBR322 ori | This study |
| pSAIspS | Cam^{r}; araBAD promoter; ispS; p15A ori | This study |
| pSAHisDF₁ | Cam^{r}; T7 promoter; (His)₆ tagged ispDF₁; p15A ori | This study |
| pSAHisDF₂ | Cam^{r}; T7 promoter; (His)₆ tagged ispDF₂; p15A ori | This study |
| pSAHisDF₃ | Cam^{r}; T7 promoter; (His)₆ tagged ispDF₃; p15A ori | This study |
| pSASI | Amp^{r}; trc promoter; genes dxs and idi; pBR322 ori | This study |
| pAC-LYC | Cam^{r}; crtE, era, and crtB under endogenous | Addgene plasmid |
| | promoter; p15A ori | 53270¹⁰¹ |
| Genes | Description | Source |
| dxs | 1-deoxy-D-xylulose-5-phosphate synthase | NCBI Gene ID: |
| | | 945060 |
| ispD | 2-C-methyl-D-erythritol 4-phosphate cytidylyltransferase | NCBI Gene ID: |
| | | 948269 |
| ispE | 4-(cytidine 5'-diphospho)-2-C-methyl-D-erythritol kinase | NCBI Gene ID: |
| | | 945774 |
| ispF | 2-C-methyl-D-erythritol 2,4-cyclodiphosphate synthase | NCBI Gene ID: |
| | | 945057 |
| idi | isopentenyl-diphosphate Delta-isomerase | NCBI Gene ID: |
| | | 949020 |
| ispDF₁ | Codon optimized bifunctional 2-C-methyl-D-erythritol 4- | This study, |
| | phosphate cytidylyltransferase/2-C-methyl-D-erythritol 2,4- | USPTO |
| | cyclodiphosphate synthase (NR0032_N05) | PCT/CA2018/05 |
| | | 1073 |
| ispDF₂ | Codon optimized bifunctional 2-C-methyl-D-erythritol 4- | This study, |
| | phosphate cytidylyltransferase/2-C-methyl-D-erythritol 2,4- | USPTO |
| | cyclodiphosphate synthase (NR0032_O07) | PCT/CA2018/05 |
| | | 1073 |
| ispDF₃ | Codon optimized bifunctional 2-C-methyl-D-erythritol 4- | This study, |
| | phosphate cytidylyltransferase/2-C-methyl-D-erythritol 2,4- | USPTO |
| | cyclodiphosphate synthase (NR0037_N05) | PCT/CA2018/05 |
| | | 1073 |
| ispS | Isoprene synthase (Populus alba sp.) | UniProtKB: |
| | | Q50L36.1¹⁰² |

We constructed fusions with different linkers. The linkers used and their sequences are listed in Table 2.2. The linkers were added by PCR and generated by Gibson assembly.

TABLE 2.2
Types of linkers used in the study and their sequences

| | Polypeptide sequence | |
|---|---|---|
| Linker type | (N terminus → C terminus) | Reference |
| Flexible Linker (FL) | SLGGGGSAAA | 103, 104 |
| Rigid Linker (RL) | AEAAAKEAAAKEAAAKEAAAKEAAAKAAA | 103, 104 |
| cjIspDF Linker (CJ) | LPTPSFE | 79 |
| IspDF₁ Linker (XL) | RQRLRSAVAA | This study |

CJ and XL linkers sequences were generated by aligning sequences of respective fusion enzyme with E. coli IspD and IspF. Homology models were built for the natural as well as non-natural chimeric fusions using SWISS-MODEL server. The non-natural fusions are listed in Table 2.3. The IspD and IspF domains of IspDF₁ were also expressed separately. This was achieved by adding a stop codon (TAA) at the end of genetic sequence for domain ispD, taking out the genetic sequence for the linker, adding a RBS and a start codon (ATG) in frame with the genetic sequence for IspF. This enabled transcriptional level separation for the two domains. The genetic sequence coding for IspD domain is denoted as ispD₁ with corresponding protein as IspD₁. The genetic sequence coding for IspF domain is denoted as ispF₁ with corresponding protein as IspF₁.

TABLE 2.3
List of non-natural protein fusions

| Fusion | | N-terminal | C-terminal |
|---|---|---|---|
| Enzyme | Linker | protein/domain | protein/domain |
| IspD_{FL}F | Flexible Linker (FL) | E. coli IspD | E. coli IspF |
| IspD_{RL}F | Rigid Linker (RL) | E. coli IspD | E. coli IspF |
| IspD_{CJ}F | cjIspDF Linker (CJ) | E. coli IspD | E. coli IspF |
| IspD_{XL}F | IspDF₁ Linker (XL) | E. coli IspD | E. coli IspF |
| IspD_{FL}F₁ | Flexible Linker (FL) | IspD domain | IspF domain |
| | | of IspDF₁ | of IspDF₁ |
| IspD_{RL}F₁ | Rigid Linker (RL) | IspD domain | IspF domain |
| | | of IspDF₁ | of IspDF₁ |
| IspD_{CJ}F₁ | cjIspDF Linker (CJ) | IspD domain | IspF domain |
| | | of IspDF₁ | of IspDF₁ |
| IspD_{FL}E | Flexible Linker (FL) | E. coli IspD | E. coli IspE |
| IspE_{FL}F | Flexible Linker (FL) | E. coli IspE | E. coli IspF |

The non-natural fusions were cloned with other genes involved in the MEP pathway to assess their influence on the pathway flux. These constructs and strains are mentioned in Table 2.4.

TABLE 2.4
Strains and plasmid expressing non-natural fusion proteins

| Strains | Description |
|---|---|
| SALyc- | pSASD_{FL}FI and pAC-LYC coexpressed |
| SD_{FL}FI | in E. coli BL21(DE3) |
| SALyc- | pSASD_{RL}FI and pAC-LYC coexpressed |
| SD_{RL}FI | in E. coli BL21(DE3) |
| SALyc- | pSASD_{CJ}FI and pAC-LYC coexpressed |
| SD_{CJ}FI | in E. coli BL21(DE3) |
| SALyc- | pSASD_{XL}FI and pAC-LYC coexpressed |
| SD_{XL}FI | in E. coli BL21(DE3) |
| SALyc- | pSASD_{FL}FEI and pAC-LYC coexpressed |
| SD_{FL}FEI | in E. coli BL21(DE3) |
| SALyc- | pSASD_{RL}FEI and pAC-LYC coexpressed |
| SD_{RL}FEI | in E. coli BL21(DE3) |
| SALyc- | pSASD_{CJ}FEI and pAC-LYC coexpressed |
| SD_{CJ}EI | in E. coli BL21(DE3) |
| SALyc- | pSASD_{XL}FEI and pAC-LYC coexpressed |
| SD_{XL}FEI | in E. coli BL21(DE3) |
| SALyc- | pSASD_{FL}F₁I and pAC-LYC coexpressed |
| SD_{FL}F₁I | in E. coli BL21(DE3) |
| SALyc- | pSASD_{RL}F₁I and pAC-LYC coexpressed |
| SD_{RL}F₁I | in E. coli BL21(DE3) |
| SALyc- | pSASD_{CJ}F₁I and pAC-LYC coexpressed |
| SD_{CJ}F₁I | in E. coli BL21(DE3) |
| SALyc- | pSASD₁-F₁I and pAC-LYC coexpressed |
| SD₁F₁I | in E. coli BL21(DE3) |
| SALyc- | pSASD_{FL}F₁EI and pAC-LYC coexpressed |
| SD_{FL}F₁EI | in E. coli BL21(DE3) |
| SALyc- | pSASD_{RL}F₁EI and pAC-LYC coexpressed |
| SD_{RL}F₁EI | in E. coli BL21(DE3) |
| SALyc- | pSASD_{CJ}F₁EI and pAC-LYC coexpressed |
| SD_{CJ}F₁EI | in E. coli BL21(DE3) |
| SALyc- | pSASD₁-F₁EI and pAC-LYC coexpressed |
| SD₁F₁EI | in E. coli BL21(DE3) |
| SALyc- | pSASD_{FL}EFI and pAC-LYC coexpressed |
| SD_{FL}EFI | in E. coli BL21(DE3) |
| SALyc- | pSASE_{FL}FDI and pAC-LYC coexpressed |
| SE_{FL}FDI | in E. coli BL21(DE3) |
| Plasmids | Description |
| pSASD_{FL}FI | Amp^{r}; trc promoter; genes dxs, ispD_{FL}F and idi; pBR322 ori |
| pSASD_{RL}FI | Amp^{r}; trc promoter; genes dxs, ispD_{RL}F and idi; pBR322 ori |
| pSASD_{CJ}FI | Amp^{r}; trc promoter; genes dxs, ispD_{CJ}F and idi; pBR322 ori |
| pSASD_{XL}FI | Amp^{r}; trc promoter; genes dxs, ispD_{XL}F and idi; pBR322 ori |
| pSASD_{FL}FEI | Amp^{r}; trc promoter; genes dxs, ispD_{FL}F, |
| | idi and ispE; pBR322 ori |
| pSASD_{RL}FEI | Amp^{r}; trc promoter; genes dxs, ispD_{RL}F, |
| | idi and ispE; pBR322 ori |
| pSASD_{CJ}FEI | Amp^{r}; trc promoter; genes dxs, ispD_{CJ}F, |
| | idi and ispE; pBR322 ori |
| pSASD_{XL}FEI | Amp^{r}; trc promoter; genes dxs, |
| | ispD_{XL}F, idi and ispE; pBR322 ori |
| pSASD_{FL}F₁I | Amp^{r}; trc promoter; genes dxs, ispD_{FL}F and idi; pBR322 ori |
| pSASD_{RL}F₁I | Amp^{r}; trc promoter; genes dxs, ispD_{RL}F₁ and idi; pBR322 ori |
| pSASD_{CJ}F₁I | Amp^{r}; trc promoter; genes dxs, ispD_{CJ}F₁ and idi; pBR322 ori |
| pSASD₁-F₁I | Amp^{r}; trc promoter; genes dxs, ispD1, |
| | ispF₁ and idi; pBR322 ori |
| pSASD_{FL}F₁EI | Amp^{r}; trc promoter; genes dxs, ispD_{FL}F, |
| | idi and ispE; pBR322 ori |
| pSASD_{RL}F₁EI | Amp^{r}; trc promoter; genes dxs, |
| | ispD_{RL}F₁, idi and ispE; pBR322 ori |
| pSASD_{CJ}F₁EI | Amp^{r}; trc promoter; genes dxs, ispD_{CJ}F₁, |
| | idi and ispE; pBR322 ori |
| pSASD₁-F₁EI | Amp^{r}; trc promoter; genes dxs, ispD₁, |
| | ispF₁, idi and ispE; pBR322 ori |
| pSASD_{FL}EFI | Amp^{r}; trc promoter; genes dxs, ispD_{FL}E, |
| | idi and ispF; pBR322 ori |
| pSASE_{FL}FDI | Amp^{r}; trc promoter; genes dxs, ispE_{FL}F, |
| | idi and ispD; pBR322 ori |

Both isoprene and lycopene starter cultures were cultivated overnight at 30° C. in LB media (Sigma-Aldrich) containing appropriate antibiotic/s. Isoprene starter cultures were then diluted to 15 mL with the medium to OD600 of 0.2, induced with arabinose and/or IPTG; and allowed to grow for 24 h at 30° C. in 25 mL sealed glass tube. Lycopene starter cultures were diluted to 5 mL with the medium to OD600 of 0.2, induced with IPTG, and allowed to grow for 24 h at 30° C. in culture tubes in the dark.

Isoprene analysis was performed on PerkinElmer Clarus 680 gas chromatograph and Perking Elmer Clarus SQ 8 T mass spectrometer (GC-MS). Since isoprene is volatile monoterpene, the sealed cultures were heated at 70° C. for 1 min and vortexed for 5 sec before sampling 200 μL of headspace using a gas-tight syringe. The standard curve for isoprene was prepared in a similar manner for quantification. HP-5MS capillary column (25 m long, 0.2 mm internal diameter, 0.33 μm film thickness; Agilent Technologies) was used, with helium (1 mL/min) as a carrier gas. The oven temperature program was 35° C. for 3 min, 25° C./min to 200° C. and hold for 1 min. The injector was maintained at 60° C. and 20:1 split ratio was maintained Mass spectrum acquisition was carried out in SIR mode for m/z 68 and m/z 67 ions.

Lycopene is an intracellular product. 2 mL of cell culture was centrifuged at 8000 rpm for 5 min and lycopene was extracted by extraction from the pellet with 1 mL acetone. Extraction was performed at 55° C. with intermittent vortexing for 20 min in reduced light condition. The acetone suspension was centrifuged and filtered before analysis. Samples were analyzed on the PerkinElmer Flexar system equipped with Zorbax C-18 column (4.6×250 mm, Agilent Technologies) maintained at 30° C. Samples were run with mobile phase consisting of 66% (v/v) methanol, 30% (v/v) tetrahydrofuran and 4% (v/v) water at 1 mL/min flow rate. Lycopene detection was done by monitoring absorbance at 474 nm wavelength using a photodiode detector.

### Results

Soil metagenome sequences were screened for higher active and stable orthologs of MEP pathway enzymes. This led to the discovery of novel fusions of two enzymes in the pathway—IspD and IspF. They were isolated from fosmids NR0032 N05, NR0032_007 and NR0037_N05 and the corresponding genes were annotated as ispDF₁, ispDF₂ and ispDF₃ respectively. The translated polypeptides were annotated as IspDF₁ (41.6 kDa), IspDF₂ (42.1 kDa) and IspDF₃ (40.2 kDa) respectively. These genes were tagged for affinity-based separation and expressed in E. coli BL21(DE3) using 0.5 mM IPTG as an inducer. Desired bands were seen on SDS-PAGE gel but the expression levels of IspDFs were low. Insoluble cell debris were denatured and analyzed, and it was realized that all three fusions formed inclusion bodies.

Sequences of IspDF₁, IspDF₂ and IspDF₃ were aligned with E. coli IspD, IspF and cjIspDF (Table 2.5). The discovered enzymes were more similar to the native monofunctional enzymes in E. coli. When aligned against cjIspDF⁷⁹, more differences were observed. Though most of the residue functions were conserved among all five ( ), the dissimilarity existed in clusters. The amino acid region between 220 and 250 residues was highly variable and was involved in linking both the domains. Other dissimilar clusters were observed in the IspD domain of the fusion. All three IspDFs discovered have novel sequence and are not reported.

TABLE 2.5
Protein alignment analysis of the bifunctional enzymes against
E. coli IspD-IspF and cjIspDF using the online BLASTN search tool

| | % Query | % Sequence | % Query | % Sequence |
|---|---|---|---|---|
| | cover when | similarity | cover | similarity |
| | aligned | when | when | when |
| | with | aligned | aligned | aligned |
| Bifunctional | E. coli | with E.coli | with | with |
| enzymes | IspD, IspF | IspD, IspF | cjIspDF | cjIspDF |
| IspDF₁ | 97 | 40.81 | 94 | 29.71 |
| IspDF₂ | 99 | 40.72 | 97 | 29.75 |
| IspDF₃ | 98 | 41.60 | 99 | 32.20 |

Each domain of the fusion enzymes was aligned against E. coli IspD and E. coli IspF (Table 2.6). The IspF domains of the fusions share greater sequence similarity with E. coli IspF than the similarity between IspD domain and E. coli IspD. This observation is consistent with the similarity reported for cjIspDF with E. coli native enzymes⁶². IspF domain of cjIspDF shares 48% sequence similarity with E. coli IspD whereas IspD domain shares 25% similarity with E. coli IspD.

TABLE 2.6
Protein alignment analysis of each domain of the
bifunctional enzymes against corresponding E. coli monofunctional
enzymes using the online BLASTN search tool

| | % Query | % Sequence | % Query | % Sequence |
|---|---|---|---|---|
| | cover when | similarity | cover when | similarity |
| | IspD | when | IspF | when IspF |
| | domain | IspD domain | domain | domain |
| Bifunctional | aligned with | aligned with E. | aligned with | aligned with |
| enzymes | E. coli IspD | coli IspD | E. coli IspF | E. coli IspF |
| IspDF₁ | 94 | 35.71 | 90 | 56.38 |
| IspDF₂ | 100 | 37.55 | 89 | 49.35 |
| IspDF₃ | 98 | 35.93 | 96 | 51.30 |

When the domains of fusions were aligned against cjIspDF domains, a similar trend was observed (Table 2.7).

TABLE 2.7
Protein alignment analysis of each domain of the bifunctional
enzymes against corresponding cjIspDF enzyme domains
using the online BLASTN search tool

| | | % Sequence | % Query | % Sequence |
|---|---|---|---|---|
| | % Query | similarity | cover when | similarity |
| | cover when | when IspD | IspF | when IspF |
| | IspD | domain | domain | domain |
| | domain | aligned | aligned with | aligned |
| | aligned with | with IspD | IspF | with IspF |
| Bifunctional | IspD domain | domain of | domain | domain of |
| enzymes | of cjIspDF | cjIspDF | of cjIspDF | cjIspDF |
| IspDF₁ | 95 | 23.77 | 86 | 42.36 |
| IspDF₂ | 97 | 24.89 | 86 | 41.72 |
| IspDF₃ | 95 | 26.89 | 95 | 43.14 |

Enzymatic steps catalyzed by Dxs, IspD, IspF and Idi are the rate-controlling steps of the MEP pathway²⁴ in E. coli. The same chassis was reconstructed (pSASDFI) and analyzed for protein expression. The soluble protein samples were run SDS/PAGE gel and stained with Coomassie dye.

SASDFI was tested for activity towards isoprene and lycopene production by co-expressing the chassis with downstream pathway (pSAIspS and pAC-LYC respectively). The clone expressing Dxs and Idi (pSASI) was constructed to account for the influence of IspD and IspF on MEP pathway flux improvement.

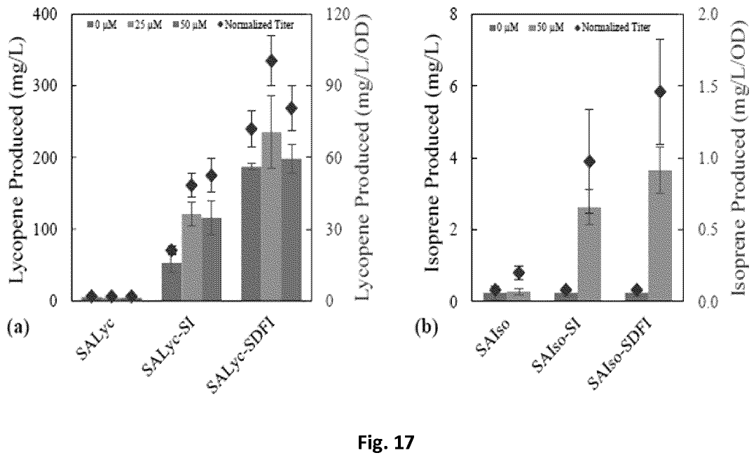

SALyc and SAIso made the corresponding terpenoid at very low yield (FIGS. 17 (a)-(b)). These strains reflect the native expression level of the MEP pathway. Induction did not have a substantial influence on terpenoid production. IPTG induction for SAIso had a negative impact on cell growth and hence shows higher normalized yield. Higher IPTG induction levels were detrimental to lycopene production and had a negative influence on growth. Overexpression of Dxs and Idi (strains SALyc-SI and SAIso-SI) produced 22-fold and 12-fold more terpenoid respectively. Additional expression of IspD and IspF (strains SALyc-SDFI and SAIso-SDFI) further enhanced the terpenoid production by 47-fold and 15-fold respectively. Uninduced cultures of SALyc-SI and SALyc-SDFI still produced lycopene at a higher yield than that of SALyc.

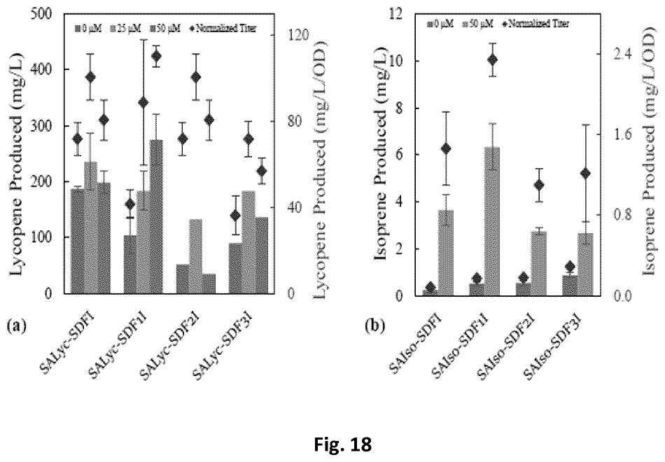

All three fusions exhibited different effects on isoprene and lycopene production (FIGS. 18 (a)-(b)). SALyc-SDF₁I and SAIso-SDF₁I were the best performers. There was 20% and 75% improvement in lycopene and isoprene production respectively for IspDF₁ strains. The IspDF₂ and IspDF₃ versions lowered the titer. OD600 for strains were in a similar range. IspDF₁ variants showed higher normalized titer which means the catalytic throughput was improved as well. SALyc-SDF₁I was tested at IPTG induction concentrations of 75 μM and 100 μM, but the titer declined, and the maximum titer was obtained at 50 μM IPTG concentration.

To assess the influence sole contribution from IspDFs, strains SAIso-DF₁, SAIso-DF₂ and SAIso-DF₃ were tested for isoprene productions; and strains SALyc-DF₁, SALyc-DF₂ and SALyc-DF₃ were tested for lycopene production. All these six strains made respective terpenoid in the levels equal to SAIso and SALyc (data not shown). The induction had no effect on the terpenoid titer.

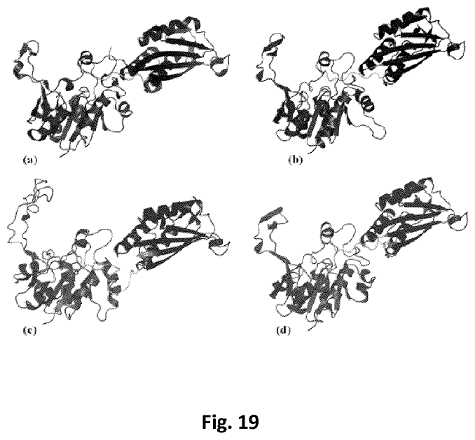

Homology models for the fusions were generated by SWISS-MODEL using cjIspDF as a template (FIGS. 19 (a)-(d)). All four fusions have conserved subunit structures. IspDF₁ and IspDF₃ align well with cjIspDF but IspDF₂ has a longer linker. The active sites of the subunits are located at opposite ends. The putative linker sequences are: EAIARGTGERAVGERAA for IspDF₂ and ERLIGARNTAGAM for IspDF₃. Since, IspDF₁ improved the terpenoid titer, it was used for further study.

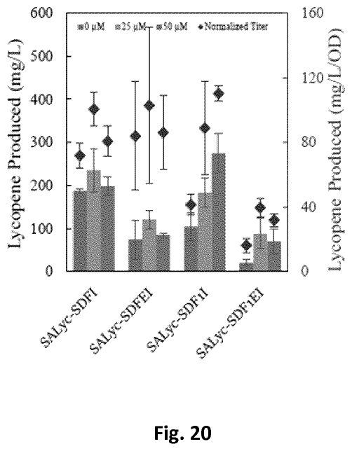

Since, IspE is reported to influence the flux by associating with IspD and IspF⁶². The association complex then assists efficient transfer and conversion of metabolites from MEP to MEcPP. We investigated this phenomenon for lycopene production by testing the recombinant E. coli strain expressing five enzymes Dxs, IspD, IspF (or IspDF), IspE and Idi. For both SALyc-SDFEI and SALyc-SDF1EI had lower lycopene titers than SALyc-SDFI and SALyc-SDF₁I respectively (FIG. 20). The percent loss in flux on IspE overexpression was more evident for IspDF₁ clone than monofunctional native enzyme clone. This effect was a summation of the lower rate of lycopene as well as lower cell growth rate. The OD600 in IspE clones was remarkably lower (by 20-60). SALyc-SDFEI cultures had higher variable growth reflecting in wider error bars.

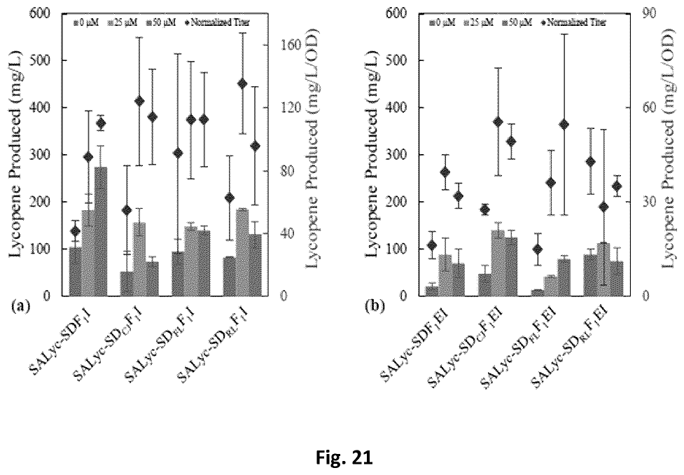

To evaluate the role of the linker in the enhancement of flux in SALyc-SDF₁I, we replaced the putative linker sequences with three types linkers. First is the linker identified from cjIspDF. Second is ‘FL’ that is glycine and serine linker and imparts flexibility to the domains. The third is ‘RL’ that forms an α-helix and restricts the free movement and giving rigidity to the conformation. The effect of the linker was tested in strains with and without IspE overexpression. The non-natural linkers did not improve the overall titers of lycopene (FIGS. 21(a)-(b)) but influenced cell viability and lowered OD600 for the cultures. Normalized titers were highest for SALyc-SD_{RL}F₁I followed by SALyc-SD_{CJ}F₁I. The clone with flexible linker displayed lowest lycopene titers in both the sets.

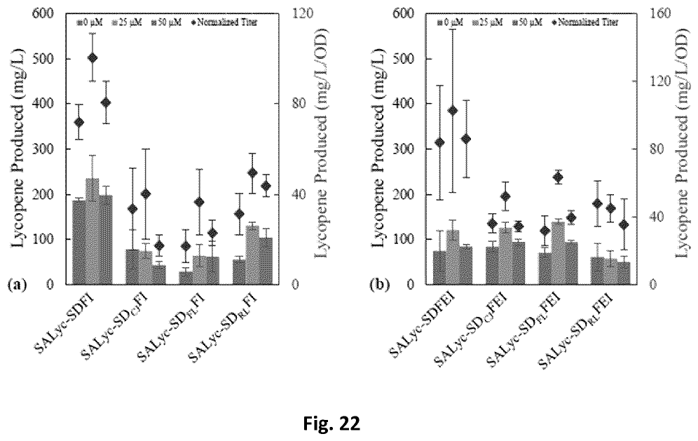

Linkers in section above had a positive impact on the normalized titers. This means that the linkers improved the flux at the cost of cell growth. The same linkers along with the natural linker of IspDF₁ were then employed to link E. coli IspD and IspF. For strains in FIG. 22(b), the lower normalized titers were the result of higher OD600. This suggests overall carbon flux channeling towards cell growth metabolisms. Whereas, for strains depicted in FIG. 22(a), the fusions had a negative impact on lycopene products without the substantial effect of cell growth.

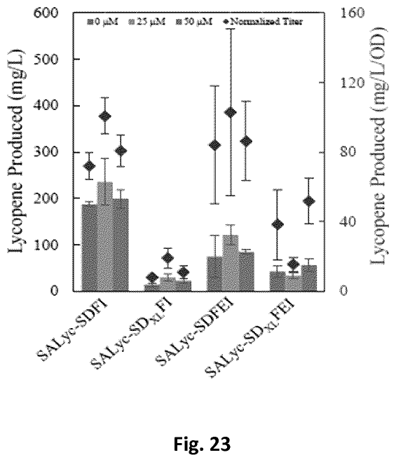

Since the strains exhibited a mixed response to CJ, FL and RL linkers, fusions of IspD and IspF with the putative linker of IspDF₁ were constructed. These fusions lowered the MEP flux and further decreased the lycopene production (FIG. 23). This effect was pronounced for SALyc-SD_{XL}FI. SALyc-SD_{XL}FEI.

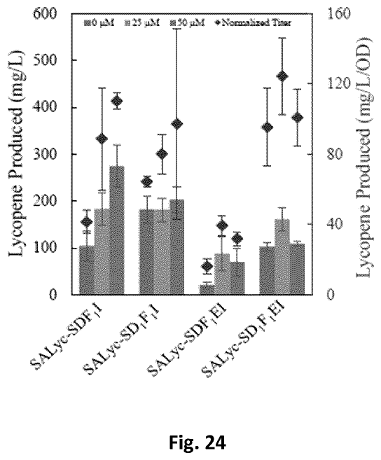

The XL linker's negative impact on the pathway flux suggested the need to study the domains of IspDF₁ in isolation (FIG. 24). The separation of the domains as individual enzymes had a more pronounced effect on SALyc-SD₁F₁EI.

Non-Natural Fusions of IspE and their Effects on MEP Pathway Flux

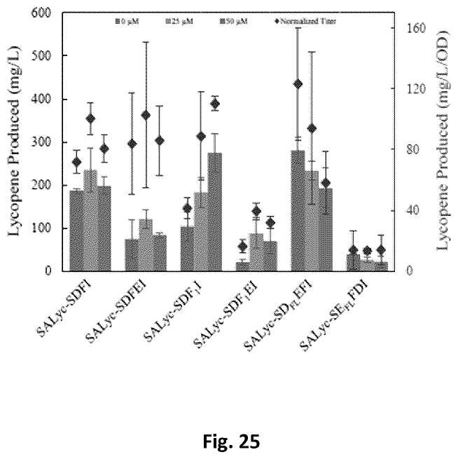

To evaluate the cause behind the natural existence of fusions of enzymes that catalyze non-consecutive steps in the MEP pathway, we constructed non-natural fusions of IspE. The fusions were constructed using the flexible linker. The linking strategy was kept similar to that of natural IspDFs. The IspDE fusion was constructed by linking C-terminus of IspD to N-terminus of IspE. And, the IspEF fusion was constructed by linking C-terminus of IspE to N-terminus of IspF. FIG. 25 shows that IspDE fusion exhibited a 20% improvement in lycopene production compared to SALyc-SDFI and 2.3-fold improvement than SALyc-SDFEI. Whereas, IspEF fusion lowered the lycopene production substantially.

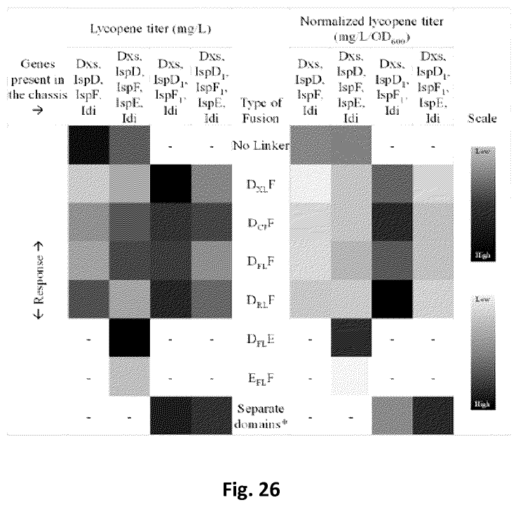

FIG. 26 summarizes the results obtained so far. It is a comparison plot for different constructs with the highest titer and normalized titer values. The blank places denoted by ‘-’.

### Discussion

The lycopene production chassis is under the control of an endogenous promoter and MEP pathway chassis is under the control of trc promoter that is reported to be leaky¹⁰⁵⁻¹⁰⁷. Due to these reasons, lycopene cultures at no induction produced higher lycopene than that of the base strain SALyc. Higher normalized titers in both lycopene and isoprene fermentation indicate abundance of C₅ precursor metabolites—IPP and DMAPP that are shuttled to respective downstream terpene synthesis pathway.

To study fusions and role of linkers, it was necessary to construct the basal chassis overexpressing Dxs, IspD, IspF and Idi that was reported to increase the taxol yield²⁴. This strain containing plasmid pSASDFI served as the basis for comparison in this study. Some reports emphasized overexpression of Dxs and Idi only for improvement of MEP pathway flux^{108,109} and results of this study (FIG. 17) showed that additional overexpression of IspD and IspF improved the titers for lycopene by 80% and that of isoprene but 35%. The micro-aerobic environment during isoprene cultures could be responsible for the disparity in titers as it is highly oxygen-limited environment. The Lycopene titers obtained in SALyc-SDFI are comparable to the titers reported in literature^{110,111}. Overall, the pSADFI chassis improved lycopene production by 47-fold and isoprene titers by 15-fold compared to pSALyc and pSAIso strains; and the strategy proved to be effective in eliminating bottlenecks in the MEP pathway.

Dxs is a gatekeeper gene in the MEP pathway and Idi catalyzes the terminal step maintaining equilibrium in IPP and DMAPP concentrations required for the downstream pathway of terpenoid biosynthesis. Hence, the chassis overexpressing only IspD and IspF as well as IspDFs did not influence the terpenoid titers. Production of terpenoids by SAIso-DF₁, SAIso-DF₂, SAIso-DF₃, SALyc-DF₁, SALyc-DF₂ and SALyc-DF₃ were not significantly different than the strains with no MEP pathway overexpression (data not shown). Hence it was decided to include genes dxs and idi in further experiments to study the influence of intermediary steps.

Improvement in the flux through the pathway due to IspDF₁ expression in pSASDF₁I operon can be attributed to the role of the linker imparting physical features (like flexibility or catalytic site proximity/substrate channeling) to the catalytic domains; and/or, higher stability and/or activity of IspDF₁ than the native monofunctional enzymes. The IspF domain of IspDF₁ has the highest similarity to the E. coli IspF than that of IspDF₂ and IspDF₃. The intensity of influence of IspDF₁ overexpression in lycopene strain was different than the isoprene strain. Since, IspE catalyzes the step between IspD and IspF, further investigation was carried out to evaluate the role of IspE in the catalytic cascade. IspE catalyzed step is not reported to be the bottleneck in the pathway and its overexpression exerted metabolic stress and lowered the lycopene titers. The stress effect was dominant in SALyc-SDF₁EI even though it expressed only 4 recombinant proteins versus 5 recombinant proteins in SALyc-SDFEI. This result highlighted the existence of factor/s other metabolic stress.

The first factor studied was the role of the linker. Flexible linker was chosen to impart mobility to the domains and rigid linker was chosen that forms a long helix restricting movements of the domains. Linker from cjIspDF was employed as well. For the non-natural IspDF₁ fusion, the C flux was diverted more to the MEP pathway and away from growth resulting in higher normalized lycopene titers but lower total lycopene production. SALyc-SDRLF₁I was best performing strain with 22% higher normalized titers than SALyc-SDF₁I and 33% higher normalized titers than the basal strain SALyc-SDFI. This suggested that the rigidity in the conformation of the fusion had a positive impact of the catalytic activity. Homology modeling IspDRLF₁ was inconclusive since the templates could not accurately replicate the folding of the linker. On the other hand, when IspE was overexpressed (strain SALyc-SD_{RL}F₁EI), the production decreased by 30% and the normalized titers lowered by 80%. But the OD600 of SALyc-SD_{RL}F₁EI was 50% higher than SALyc-SDRLF₁I. Since the SALyc-SD_{RL}F₁EI expresses 4 heterologous enzymes, the effective quantities of the individual enzyme are lower than that in SALyc-SD_{RL}F₁I that expresses 3 heterologous enzymes. Hence the MEP pathway flux was lower, and the overall C flux was diverted to biomass generation.

To deduce this effect further, construction and comparison were made with non-natural fusions of E. coli IspD and IspF. In these cases (FIG. 22), co-localization of the activities had a negative impact on lycopene production as well as normalized titers. But, in these cases the overall OD600 of strains overexpressing IspE was 10-50% lower than their corresponding variants not overexpressing IspE. This prompted the involvement of IspE beyond its influence on the health and growth of the cell. Moreover, the putative linker of IspDF₁ when used to link E. coli IspD and IspF, exhibited similar effects as other non-natural fusions.

The chimeric enzyme with RL type linker so far exhibited the maximum flux through the MEP pathway at an expense of cell growth. This prompted to re-evaluate effect of linker and domain co-localization. The prevailing theory of organization of fusion of enzymes improves the rate of reaction cascade is by lowering the substrate diffusional limitations and substrate channeling. But the recent evidence show that the dynamics of fusions on a metabolic cascade is more complex than previously assumed¹¹². It is not simply the proximity between enzymes that enhances the initial reaction rate; rather, colocalization increases the local concentration of enzymes. This therefore increases the chance that a diffusing substrate will interact with an active site cavity¹¹³.

IspD₁ and IspF₁ retained the individual activities. Strain SALyc-SD₁F₁I had 25% lower lycopene production but the W600 was lower by the same factor as well. Hence the overall flux and the normalized titers were similar. SALyc-SD₁F₁EI had 30% lower OD600 and displayed a 82% increase in lycopene production. Both these observations factored to three-fold improvement in normalized lycopene titers for SALyc-SD₁F₁EI than SALyc-SDF₁EI. Though the overall lycopene titers remained lower than SALyc-SDF₁I due to lower availability of copies of enzyme because of longer operon; improvement in the normalized titers bolstered the observation of higher stability and activity of IspDF₁.

The absence of any literature on the fusion of IspE is noticeable in contrast to many discoveries of IspDFs. The role of fusion of enzymes catalyzing non-consecutive steps in the pathway and role of intermediary step enzyme is not only highly debated but also rather unforeseeable. I tried to unravel it by constructing non-natural fusions of IspE. The performance of IspDE fusion was many folds better than IspEF fusion. In fact, IspDE fusion exhibited 2.3-fold improvement in lycopene production and 20% improvement in the normalized titers than that of SALyc-SDFEI. The OD600 for SALyc-SD_{FL}EFI was doubled as well. Whereas, IspEF fusion decreased the lycopene production at least 65% normalized titers by 85% than of SALyc-SDFEI. The strain SALyc-SD_{FL}EFI was the best performing strain for lycopene production and second best in MEP pathway flux after SALyc-SD_{RL}F₁I. This was due to the fact that the individual domains of IspDF₁ are higher active than E. coli native IspD and IspF.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed.

Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those skilled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

What is claimed is:

1. A host cell comprising:
a. an expression cassette comprising a promoter operably linked to a heterologous nucleic acid encoding a heterologous transporter or a functional fragment thereof, wherein the transporter is selected from the group consisting of a major facilitator superfamily (MFS) aromatic acid antiporter and an OprD family porin; and
b. an aromatic substrate selected from olivetolate, divarinolate (DVA), or a metabolite, derivative, or decarboxylate thereof,
wherein said host cell is capable of increased import of the aromatic substrate into the host cell as compared to a control host cell that lacks the expression cassette of a).

2. The host cell of claim 1, wherein the cell is a prokaryote, preferably wherein the prokaryote selected from the group consisting of a prokaryote of the genus Escherichia, Panteoa, Bacillus, Corynebacterium, or Lactococcus.

3. The host cell of claim 1, wherein the cell is Escherichia coli (E. coli), Panteoa citrea, C. glutamicum, Bacillus subtilis, or L. lactis.

4. The host cell of claim 1, wherein the cell is Escherichia coli (E. coli).

5. The host cell of any one of claims 1-4, wherein the transporter is the MFS aromatic acid antiporter pcaK or a functional fragment thereof; or wherein the transporter is the OprD family porin pp3656 or a functional fragment thereof.

6. The host cell of any one of claims 1-5, wherein the transporter is at least 50% or 55% identical to, or identical to, 100 contiguous amino acids of the sequence set forth in: SEQ ID NO. 6, 7, 8, or 9.

7. The host cell of any one of claims 1-6, wherein the host cell further comprises a heterologous aromatic prenyltransferase or functional fragment thereof, wherein the aromatic prenyltransferase is functional and capable of prenylating the aromatic acid substrate.

8. The host cell of claim 7, wherein the heterologous aromatic prenyltransferase is CBGAS or NphB or a functional fragment thereof.

9. The host cell of claim 8, wherein the heterologous aromatic prenyltransferase is a functional fragment of CBGAS.

10. The host cell of claim 9, wherein the functional fragment of CBGAS is at least 50% or 55% identical to, or identical to, 100 contiguous amino acids of the sequence set forth in SEQ ID NO. 3.

11. The host cell of any one of claims 1-10, wherein the host cell comprises an expression cassette comprising a promoter operably linked to a nucleic acid encoding one or more MEP pathway enzymes selected from the group consisting of dxs, ispC, ispD, ispE, ispF, ispDF, ispG, ispH, and idi, or a variant thereof (e.g., a variant that is at least 90%, 95%, or 99% identical to a respective native prokaryotic sequence).

12. The host cell of any one of claims 1-11, wherein the host cell comprises an expression cassette comprising a promoter operably linked to a nucleic acid encoding ispDF.

13. The host cell of any one of claims 1-12, wherein the host cell comprises an expression cassette comprising a promoter operably linked to a nucleic acid encoding ispDE.

14. The host cell of any one of claims 1-13, wherein the host cell comprises an expression cassette comprising a promoter operably linked to a nucleic acid encoding GPP synthase.

15. The host cell of any one of claims 1-14, wherein the host cell is in a culture medium comprising olivetolate, DVA, olivetol, or divarinol, preferably wherein the host cell is in a culture medium comprising olivetolate and/or DVA.

16. The host cell of any one of claims 1-15, wherein the host cell further comprises an expression cassette comprising a promoter operably linked to a nucleic acid encoding a cannabinoid synthase.

17. The host cell of claim 16, wherein the cannabinoid synthase is a CBDA synthase, CBCA synthase, or THCA synthase, preferably wherein the cannabinoid synthase is a CBDA synthase.

18. A method of increasing the transport of olivetolate into a prokaryotic host cell, the method comprising culturing a host cell according to any one of claims 1-17 in culture media containing exogenous aromatic substrate of the transporter under conditions suitable to express the transporter.

19. A method of prenylating olivetolate and/or DVA, the method comprising culturing a host cell according to any one of claims 7-17 in culture media containing exogenous olivetolate and/or DVA under conditions suitable to express the transporter and the aromatic prenyltransferase, thereby prenylating the olivetolate and/or DVA.

20. The method of claim 19, wherein the aromatic prenyltransferase is a geranyl-diphosphate:olivetolate geranyltransferase, and the method comprises producing cannabigerolic acid.

21. The method of any one of claims 19 to 20, wherein the method increases the production of a prenylated olivetolate or DVA product as compared to a control method performed under conditions that do not express, or express a lower amount or activity of, the transporter.

22. The method of any one of claims 19 to 21, wherein the method comprises harvesting and lysing the cultured cell, thereby producing cell lysate.

23. The method of claim 22, wherein the method comprises purifying the prenylated olivetolate or DVA product, or a metabolite thereof, from the cell lysate.

24. The method of any one of claims 19 to 21, wherein the method comprises harvesting spent culture medium produced by culturing the host cell.

25. The method of claim 24, wherein the method comprises purifying the prenylated olivetolate or DVA product, or a metabolite thereof, from the spent culture medium.

26. The method of claim 23 or 25, wherein the method comprises purifying CBGA, or a decarboxylation product thereof, from the cell lysate or spent culture medium.

27. The method of claim 21 or 25, wherein the method comprises purifying CBDA, or a decarboxylation product thereof, from the cell lysate or spent culture medium.

28. An expression cassette comprising a heterologous promoter operably linked to a nucleic acid encoding a bifunctional ispDE enzyme or functional fragment thereof.

29. An expression cassette comprising a heterologous promoter operably linked to a nucleic acid encoding a bifunctional ispDE, ispDF, or ispEF enzyme or a functional fragment thereof, preferably wherein the nucleic acid encodes a bifunctional ispDE enzyme or functional fragment thereof.

30. The expression cassette of claim 28, wherein the bifunctional ispDE enzyme comprises a sequence at least 80% identical to the sequence set forth in SEQ ID NO:10.

31. The expression cassette of claim 28, 29 or 30, wherein the expression cassette comprises a promoter operably linked to a nucleic acid encoding at least one additional MEP pathway enzyme.

32. The expression cassette of claim 30, wherein said at least one additional MEP pathway enzyme comprises:
a. dxs, ispF and idi, or
b. dxs, ispDF, and idi.

33. A host cell comprising the expression cassette of any one of claims 28 to 32.

34. The host cell of claim 33, wherein the host cell further comprises an expression cassette comprising a promoter operably linked to a nucleic acid encoding a terpenoid synthase.

35. The host cell of claim 33 or 34, wherein the host cell further comprises an expression cassette comprising a promoter operably linked to a nucleic acid encoding a cannabinoid synthase.

36. The host cell of any one of claims 33-35, wherein the host cell further comprises an expression cassette comprising a promoter operably linked to a nucleic acid encoding an aromatic prenyltransferase.

37. The host cell of any one of claims 33-36, wherein the host cell further comprises an expression cassette comprising a promoter operably linked to a nucleic acid encoding GPP synthase.

38. The host cell of any one of claims 33-37, wherein the host cell comprises the nucleic acid encoding ispDE, the nucleic acid encoding the GPP synthase, the nucleic acid encoding the aromatic prenyltransferase, and the nucleic acid encoding a cannabinoid synthase selected from the group consisting of CBDA synthase or a functional fragment thereof, CBCA synthase or a functional fragment thereof, and THCA synthase or a functional fragment thereof, preferably wherein the nucleic acid encoding the cannabinoid synthase encodes CBDA synthase or a functional fragment thereof.

39. The host cell of any one of claims 33 to 38, wherein the host cell further comprises olivetolate, olivetol, divarinolic acid, or divarinol.

40. The host cell of claim 39, comprising olivetolate or divarinolic acid.

41. The host cell of claim 40, comprising olivetolate.

42. The host cell of any one of claims 33 to 41, wherein the host cell further comprises a heterologous expression cassette comprising a promoter operably linked to at least one prokaryotic chaperone.

43. The host cell of any one of claims 33 to 42, wherein the host cell comprises:
a. a heterologous nucleic acid encoding ispDF and, optionally, a heterologous nucleic acid encoding ispE;
b. a heterologous nucleic acid encoding ispDE and, optionally, a heterologous nucleic acid encoding ispF; or
c. a heterologous nucleic acid encoding ispEF and, optionally, a heterologous nucleic acid encoding ispD.

44. The host cell of any one of claims 33 to 43, wherein at least one, at least two, at least three, at least four, or all heterologous expression cassettes are integrated into the genome of the host cell.

45. The host cell of any one of claims 33 to 43, wherein at least one of the expression cassettes is not integrated into the genome of the host cell.

46. A method of producing a terpenoid, the method comprising culturing a hot cell of any one of claims 33 to 45 under conditions suitable to express the ispDE bifunctional enzyme.

47. The method of claim 46, wherein the method comprises culturing the host cell in culture media comprising an exogenously supplied substrate of a heterologously expressed aromatic prenyltransferase.

48. The method of claim 47, wherein the exogenously supplied substrate comprises olivetolate or divarinolic acid, preferably olivetolate.
